(19) United States
(12) Patent Application Publication
(10) Pub. No.: US 2022/0226269 A1
(43) Pub. Date: Jul. 21, 2022

(54) METHODS AND COMPOSITIONS FOR MODULATION OF AN INTERSPECIES GUT BACTERIAL PATHWAY FOR LEVODOPA METABOLISM

(71) Applicant: President and Fellows of Harvard College, Cambridge, MA (US)

(72) Inventors: Emily Balskus, Somerville, MA (US); Vayu Maini Rekdal, Somerville, MA (US)

(21) Appl. No.: 17/618,334

(22) PCT Filed: Jun. 12, 2020
(86) PCT No.: PCT/US2020/037429

§ 371 Date: Dec. 10, 2021

Related U.S. Application Data
(60) Provisional application No. 62/860,468, filed on Jun. 12, 2019.

(51) Int. Cl. A61K 31/198; C12N 15/113; A61K 31/7105; A61K 38/46; A61K 39/395; A61P 25/16

(52) U.S. Cl. CPC A61K 31/198; C12N 15/1137; A61K 31/7105; A61K 38/465; C12N 2310/141; A61P 25/16; C12N 2310/14; C12N 2320/31; A61K 39/3955

(57) ABSTRACT

The present disclosure relates to the use of an agent that inhibits the activity of or decreases the levels of an L-dopa decarboxylase conjointly with levodopa (L-dopa) in the treatment of a condition.

### RELATED APPLICATIONS

This application claims priority to U.S. Provisional Application No. 62/860,468, filed Jun. 12, 2019, which is incorporated herein by reference in its entirety.

### BACKGROUND

The primary treatment for Parkinson's disease is Levodopa (L-dopa), which is prescribed to manage motor symptoms resulting from dopaminergic neuron loss in the substantia nigra. After crossing the blood-brain barrier, L-dopa is decarboxylated by aromatic amino acid decarboxylase (AADC) to give dopamine, the active therapeutic agent. However, dopamine generated in the periphery by AADC cannot cross the blood-brain barrier, and only 1-5% of L-dopa reaches the brain due to extensive pre-systemic metabolism in the gut by enzymes including AADC. Peripheral production of dopamine also causes gastrointestinal side effects, can lead to orthostatic hypotension through activation of vascular dopamine receptors, and may induce cardiac arrhythmias. To decrease peripheral metabolism, L-dopa is co-administered with AADC inhibitors such as carbidopa. Despite this, 56% of L-dopa is metabolized peripherally, and patients display highly variable responses to the drug, including loss of efficacy over time.

### SUMMARY

Provided herein are methods and compositions related to treating a condition in a subject comprising administering an agent that inhibits the activity of or decreases the levels of L-dopa decarboxylase (e.g., microbial L-dopa decarboxylase). In some embodiments, the agent is administered conjointly with levodopa (L-dopa). Also provided herein are methods of treating Parkinson's Disease in a subject and/or method of treating or preventing symptoms resulting from dopaminergic neuron loss in a subject in need thereof by administering an agent to the subject that inhibits the activity of or decreases the levels of tyrosine decarboxylase (TyrDC) conjointly with levodopa.

The L-dopa decarboxylase may be TyrDC. In some embodiments, the agent preferentially inhibits the activity of or decreases the level of TyrDC over amino acid decarboxylase (AADC). In some embodiments, the condition is Parkinsonism, Parkinson's disease, corticobasal degeneration (CBD), dementia with Lewy bodies (DLB), essential tremor, multiple system atrophy (MSA), progressive supranuclear palsy (PSP), vascular (arteriosclerotic) parkinsonism, Parkinson's-like symptoms that develop after encephalitis, injury to the nervous system caused by carbon monoxide poisoning, or injury to the nervous system caused by manganese poisoning.

The agent may be a small molecule (e.g., (S)-a-Fluoromethyltyrosine (AFMT)), an interfering nucleic acid specific for a RNA product of a gene encoding TyrDC or fragment thereof, antibody or antibody fragment specific for a TyrDC protein, or a peptide that specifically binds to a TyrDC protein or fragment thereof.

The methods provided herein may include administering the agent and levodopa in one composition. For example, provided herein are compositions that comprise the agent and levodopa. In some embodiments, the agent and levodopa are in the same or different compositions. In some embodiments, the agent and the levodopa are administered simultaneously. In some embodiments, the agent and the levodopa may be administered sequentially.

The methods included herein may also comprise further administering carbidopa and/or benserazide to the subject. The method may comprise administering an agent to the subject that inhibits the activity of or decreases the levels of an enzyme that dehydroxylates dopamine (e.g., bis-molybdopterin guanine dinucleotide cofactor (moco)-containing enzyme). The method may comprise administering an agent to the subject that increases the activity of or increases the levels of an enzyme that dehydroxylates dopamine.

In some embodiments, the agent preferentially inhibits the activity of or decreases the level of a TyrDC protein over an AADC protein.

In some embodiments, the agent decreases the level of or inhibits the activity of a TyrDC protein by at least 10%, by at least 20%, by at least 30%, by at least 40%, by at least 50%, by at least 75%, by at least 90%, or by at least 99%.

The agent may be administered to the subject systemically, intravenously, subcutaneously, intramuscularly.

Also provided herein are in-vitro methods of determining whether an agent is a therapeutic agent for Parkinson's Disease comprising determining whether the test agent decreases the levels of or inhibits the activity of a TyrDC enzyme, wherein the test agent is determined to be a therapeutic agent for the treatment of Parkinson's Disease if the test agent decreases the levels of or inhibits the activity of a TyrDC enzyme. The test agent may be a member of a library of test agents. The test agent may be an interfering nucleic acid, a peptide, a small molecule, or an antibody. The agent may decrease the level of or inhibits the activity of the TyrDC enzyme by at least 10%, at least 20%, at least 30%, at least 40%, at least 50%, at least 75%, or at least 95%.

Provided herein are methods of treating a condition in a subject by administering a composition that inhibits the activity of or decreases the levels of a bacteria that expresses a PLP-dependent tyrosine decarboxylase (TyrDC) or a TyrDC homolog conjointly with levodopa. The bacteria that expresses a PLP-dependent tyrosine decarboxylase may be Enterococcus faecalis. The bacteria may express a PLP-dependent tyrosine decarboxylase may be Enterococcus faecium. The bacteria that expresses a PLP-dependent tyrosine decarboxylase may be Lactobacilli. The bacteria that expresses a PLP-dependent tyrosine decarboxylase may be Providencia (e.g., Providencia rettgeri). The bacteria that expresses a PLP-dependent tyrosine decarboxylase may be Proteus (e.g., Proteus mirabilis). In some embodiments, the condition is Parkinsonism, Parkinson's disease, corticobasal degeneration (CBD), dementia with Lewy bodies (DLB), essential tremor, multiple system atrophy (MSA), progressive supranuclear palsy (PSP), vascular (arteriosclerotic) parkinsonism, Parkinson's-like symptoms that develop after encephalitis, injury to the nervous system caused by carbon monoxide poisoning, or injury to the nervous system caused by manganese poisoning.

The method may further comprise administering a composition that inhibits the activity of or decreases the levels of bacteria that expresses a molybdenum-dependent enzyme. In some embodiments, the bacteria that expresses a molybdenum-dependent enzyme is Eggerthella lenta.

### BRIEF DESCRIPTION OF THE DRAWINGS

FIG. 1 has six panels, A-F, and shows Enterococcus faecalis metabolizes L-dopa using a pyridoxal phosphate (PLP)-dependent tyrosine decarboxylase. Panel A shows proposed major pathway for L-dopa metabolism by the human gut microbiota and potential for interaction with host-targeted drugs. Panel B shows phylogenetic distribution of tyrosine decarboxylases (TyrDC) in the human microbiota. Human Microbiome Project reference genomes were queried by BLASTP for homologs of the L. brevis TyrDC, and the results are visualized on a cladogram of phylogeny (based on 16S rRNA alignment). TyrDC homologs found sporadically within Lactobacillus spp. (Lb) are widely distributed amongst Enterococcus (Ec; average amino acid identity 67.8% over 97.6% query length). Panel C shows testing representative gut microbial strains encoding TyrDC reveals that E. faecalis strains reproducibly convert L-dopa to dopamine. Strains were cultured for 48 hours anaerobically. Bar graphs represent the mean±the standard error of the mean (SEM) of three biological replicates. Panel D shows deletion of tyrDC abolishes L-dopa decarboxylation by E. faecalis. Dopamine was detected in culture supernatants after 48 hours of anaerobic growth with 0.5 mM L-dopa. Bar graphs represent the mean±the SEM of three biological replicates. Panel E shows kinetic analysis of E. faecalis TyrDC reveals a preference for tyrosine. Error bars represent the mean±the SEM of three biological replicates. ND=not detected. Panel F shows L-dopa and tyrosine are simultaneously decarboxylated in anaerobic cultures of E. faecalis MMH594 grown at pH 5 with 1 mM L-dopa and 0.5 mM tyrosine. Bar graphs represent the mean±the standard error of the mean (SEM) of three biological replicates.

FIG. 2 has four panels, A-D and shows Eggerthella lenta dehydroxylates dopamine using a molybdenum-dependent enzyme. Panel A shows RNA-Seq identifies a putative molybdenum (moco)-dependent dopamine dehydroxylase (Dadh) in E. lenta A2. Differentially expressed candidate genes (FDR<0.1 and fold-change (FC)>|2|) are plotted as a function of genome position revealing three discrete loci of differentially expressed genes. (Inset) analysis of the largest cluster of differentially expressed genes at 0.665 megabase pairs (Mbp) in the scaffolded assembly (190 kilo basepairs in the reference contig) revealed the putative dadh up-regulated by 2568-fold in response to dopamine. Panel B shows tungstate treatment inhibits dehydroxylation of dopamine by E. lenta A2. Cultures were grown anaerobically with tungstate (WO₄²⁻) or molybdate (MoO₄²⁻) for 48 hours with 0.5 mM dopamine. Bar graphs represent the mean±the SEM of three biological replicates. Panel C shows in vitro activity of Dadh-containing fractions purified from E. lenta A2. Extracted LC-MS/MS ion chromatograms for simultaneous detection of dopamine and m-tyramine after 12 hours of anaerobic incubation of enzyme preparation with 500 dopamine and artificial electron donors at room temperature. Peak heights show the relative intensity of each mass and all chromatograms are shown on the same scale. Panel D shows single amino acid variant predicts dopamine metabolism by E. lenta and related strains (P=0.013 Fisher's Exact) and does not correlate with phylogeny. Strains were cultured anaerobically with 500 μM dopamine for 48 hours (El E. lenta, Es E. sinensis, Gs Gordonibacter sp., Gp Gordonibacter pamelaeae). High (100% conversion) and low (<11% conversion) metabolizers are denoted in red and blue. For each strain, data points represent biological replicates. *P<0.05 ANOVA with Dunnett's test vs sterile controls.

FIG. 3 has seven panels, A-G, and shows E. faecalis and E. lenta Dadh predict L-dopa metabolism in complex human gut microbiotas. Panel A shows metabolism of L-dopa by co-cultures of E. faecalis and E. lenta strains co-cultured for 48 hours with 1 mM d₃-phenyl-L-dopa or 1 mM dopamine. Results are mean±the SEM (n=3 replicates). Panel B shows metabolism of d₃-phenyl-L-dopa by 19 unrelated human gut microbiota samples ex vivo. Samples were cultured anaerobically with d₃-phenyl-L-dopa (1 mM) for 72 hours. Results are mean concentration±the SEM (n=3 replicates). Panel C shows the abundance of tyrDC predicts L-dopa decarboxylation in human gut microbiota samples. Data represent the average tyrDC abundance (as assessed by qPCR) across the 3 replicates for samples in (Panel B). Results are mean±the SEM. ****P<0.0001, one tailed Mann-Whitney test. Panel D shows the abundance of E. faecalis (as assessed by qPCR) predicts L-dopa decarboxylation in human gut microbiota samples. Each data point is the average abundance across 3 biological replicates for each sample shown in (Panel B). Results are mean±the SEM. ****P<0.0001, one tailed Mann-Whitney test. Panel E shows dopamine dehydroxylation by gut microbiota samples of 15 unrelated individuals. Samples were cultured for 48 hours with 0.5 mM dopamine. Bars are mean±the SEM of n=6 for low reducers (<50%) and n=9 for high reducers (>50%). ***P=0.0002, one tailed Mann-Whitney test (Panel F) Dadh abundance does not correlate with dehydroxylation by human gut microbiotas. Data represent qPCR with Dadh-specific primers. Each data point is the dadh abundance in each sample shown in Panel E. Bars represent the mean and standard error. Panel G shows Dadh sequence variants predict dopamine dehydroxylation ex vivo. Full-length dadh from each culture in Panel E was sequenced using primers specific for the region containing position 506. Samples where a mix of variants were present (n=3) were removed. Bars represent the mean and SEM (n=3 for AGC samples, n=7 for CGC samples, n=3 for DSM2243, and n=3 for A2). **P=0.0083, one tailed Mann-Whitney test, CGC samples vs. AGC samples.

FIG. 4 has nine panels, A-I, and shows L-dopa decarboxylation by E. faecalis is inhibited by (S)-α-fluoromethyltyrosine (AFMT) but not the host-targeted drug carbidopa. Panel A shows carbidopa and AFMT. Panel B shows carbidopa preferentially inhibits human amino acid decarboxylase (AADC) over TyrDC. AADC or TyrDC were incubated with inhibitor and reaction rates were measured by LC-MS/MS. % Activity represents the rate relative to a no inhibitor (vehicle) control. Results are mean±the SEM (n=3 replicates). Panel C shows activity of carbidopa and AFMT in cultures of E. faecalis grown for 16 hours anaerobically with 0.5 mM L-dopa. Error bars represent the mean±the SEM for three biological replicates. Panel D shows activity of carbidopa in a human fecal microbiota from a Parkinson's patient. The sample was cultured anaerobically with carbidopa and 1 mM d₃-phenyl-L-dopa for 72 hours. Error bars represent the mean±the SEM for three biological replicates. Panel E shows AFMT preferentially inhibits TyrDC over AADC in vitro. AADC or TyrDC were incubated with inhibitor and reaction rates were measured by LC-MS/MS. % Activity represents the rate relative to a no inhibitor (vehicle) control. Error bars represent the mean±the SEM for three biological replicates. Panel F shows detection of an AFMT-PLP covalent adduct following incubation of TyrDC or AADC with AFMT for 1 hour. The data shown is the EIC of mass for the predicted covalent adduct. Panel G shows action of AFMT in human fecal microbiotas from Parkinson's patients incubated anaerobically with AFMT and 1 mM d₃-phenyl-L-dopa for 72 hours. Error bars represent the mean±the SEM for three biological replicates. Panel H shows pharmacokinetic analysis in gnotobiotic mice colonized with E. faecalis and given L-dopa+carbidopa+AFMT demonstrates higher serum L-dopa relative to vehicle controls. Error bars represent the Mean±the SEM. Part I shows the maximum serum concentration (Cₘₐₓ) of L-dopa is significantly higher with AFMT relative to vehicle controls. Panels H, I *P<0.05, Mann-Whitney U test; n=4-5/group.

FIG. 5 shows a hylogenetic tree based on the alignment of the TyrDC protein sequences from Enterococcal HMP reference genomes. The phylogenetic tree of TyrDC was prepared using FastTree and shows that the TyrDC sequences correlate with species (abbreviated Spp). Bootstrap values are shown at each node of the tree.

FIG. 6 shows a genomic organization of the tyrDC operon in E. faecalis strains used in this study. A tBLASTn search was performed in the NCBI nucleotide collection and whole genome shotgun sequences using E. faecalis TX0104 tyrDC as the query (Uniprot ID: C0X244), and the genomic context surrounding the tyrDC gene was analyzed. This search revealed that all E. faecalis strains used in our study share the same general four-gene organization of the tyrDC operon. Homologous genes are highlighted in the same color. The text inside each gene represents the NCBI accession number for the protein coding sequence.

FIG. 7 shows PCR confirmation of the E. faecalis MMH594 tyrDC mutant. gDNA from E. faecalis OG1RF (lane 1), E. faecalis TX0104 (lane 2), E. faecalis MMH594 (lane 3), or E. faecalis MMH594 tyrDC mutant (lane 4) was amplified using primers surrounding the location of the predicted 2 kb tetracycline resistance cassette insertion. While all wild-type E. faecalis strains displayed a 500 kb amplicon representing wild-type tyrDC, the mutant displayed a 2.5 kb amplicon, representing a 2 kb insertion.

FIG. 8, has two panels, A and B, and shows anaerobic growth of wild-type (WT) E. faecalis MMH594 and a tyrDC mutant. Strains were grown anaerobically in BHI medium at 37° C. either without L-dopa (Panel A) or with 500 μM L-dopa (Panel B). WT is shown in black, while the mutant is shown in red. There was no obvious growth defect of the tyrDC mutant under these conditions. The data shown are the mean of three replicate growth experiments±standard error of the mean (SEM).

FIG. 9 shows SDS-PAGE of purified recombinant enzymes. Precision Plus Protein™ All Blue Standards (lane 1), E. faecalis MMH594 TyrDC (70.1 kDa, lane 2), H. sapiens AADC (53.9 kDa, lane 3).

FIG. 10 shows L-dopa and tyrosine competition experiment with TyrDC. TyrDC (0.15 μM) was incubated with equimolar concentrations of tyrosine and L-dopa (500 μM each) in 0.2 M pH 5.5 sodium acetate buffer at room temperature. Formation of the corresponding decarboxylation products was measured by LC-MS/MS following quenching with methanol (1:10) at specific time points. The data shown is the mean of three replicate experiments±the SEM. Error bars are not visible if they are smaller than the data points.

FIG. 11 has two panels, A and B, and shows anaerobic growth, L-dopa metabolism, and tyrosine metabolism of four E. faecalis strains across varying pH. E. faecalis MMH594, V583, TX0104, and OG1RF were grown anaerobically with 1 mM L-dopa in BHI medium (pH 5 or pH 7) at 37° C. The BHI medium contained approximately 500 μM of tyrosine. Growth (Panel A) and metabolites (Panel B) were tracked over time. Though different strains displayed variability in the rate of metabolism, L-dopa and tyrosine decarboxylation occurred simultaneously regardless of the pH. The rate of decarboxylation increased at lower pH despite less growth under this condition. The data shown are the mean of three replicate growth experiments±the SEM. Error bars are not visible if they are smaller than the data points.

FIG. 12 has two panels, A and B, and shows impact of pH and tyrosine concentration on L-dopa metabolism by E. faecalis MMH594 grown in BHI medium. E. faecalis was grown anaerobically with 1 mM L-dopa in BHI medium (pH 5 or pH 7) at 37° C. The medium was supplemented either with no tyrosine or 1 mM tyrosine, generating final concentrations of approximately 500 μM tyrosine and 1.5 mM tyrosine, respectively. Growth (Panel A) and metabolites (Panel B) were tracked over time. While higher tyrosine concentrations reduced the rate of L-dopa metabolism, L-dopa and tyrosine decarboxylation occurred simultaneously across all conditions. The rate of decarboxylation increased at lower pH despite less growth under this condition. The data shown are the mean of three replicate growth experiments±the SEM. Error bars are not visible if they are smaller than the data points.

FIG. 13 has two panels, A and B, and shows the impact of pH and tyrosine concentration on d3-phenyl-L-dopa metabolism by three human fecal samples grown in BHI medium. Fecal samples were grown with 1 mM d₃-phenyl-L-dopa in BHI medium (pH 5 or pH 7) at 37° C. The pH 7 medium was supplemented either with no tyrosine or 1 mM tyrosine, creating final concentrations of approximately 500 μM tyrosine and 1.5 mM tyrosine, respectively. Growth (Panel A) and metabolites (Panel B) were tracked over time. While tyrosine and pH had variable effects depending on the stool sample, L-dopa and tyrosine decarboxylation occurred simultaneously across all samples and conditions. The data shown are the mean of three replicate growth experiments±the SEM. Error bars are not visible if they are smaller than the data points.

FIG. 14 has two panels, A and B, and shows the arnow method for colorimetric detection of dopamine in bacterial cultures. (Panel A) BHI medium with 500 μM dopamine (1) or without dopamine (2) subjected to the colorimetric assay. Dopamine produces a striking pink color that can be quantified spectrophotometrically as absorbance at 500 nm. Panel B shows absorbance at 500 nm across a range of dopamine and m-tyramine concentrations in BHI medium subjected to the colorimetric assay. While there is a linear dose-dependent increase in absorbance with dopamine, there is no increase in the presence of m-tyramine.

FIG. 15 has two panels, A and B, and shows enrichment culturing for isolating dopamine dehydroxylating strains from complex human stool samples. Panel A shows general overview of enrichment culturing strategy. A human stool sample was inoculated into a minimal medium containing 500 μM dopamine as the electron acceptor. Anaerobic growth and passaging of cultures into fresh medium allows for enrichment of active strains (red). Panel B shows pie charts describing the bacterial genus abundance at various points of enrichment culturing as assessed by 16S rRNA sequencing (>1% abundance). Both Enterococcus sp. (black) and Eggerthella sp. (purple) were present at the end of enrichment culturing and were also present in an active, metabolizing culture picked from a colony on a plate. This culture was plated again, and a pure culture of Eggerthella lenta was obtained. The isolated, active dopamine dehydroxylating strain was named Eggerthella lenta A2.

FIG. 16 shows Eggerthella lenta A2 quantitatively and regiospecifically dehydroxylates dopamine to m-tyramine in anaerobic culture. E. lenta A2 was growth with or without 500 μM dopamine for 48 hours anaerobically in BHI medium at 37° C., and the culture supernatants were analyzed by LC-MS. Data represent LC-MS/MS ion chromatograms for simultaneous detection of dopamine and m-tyramine after 48 hours of anaerobic growth. Peak heights show the relative intensity of each mass and all chromatograms are shown on the same scale. Dopamine was completely dehydroxylated to give m-tyramine but not its regioisomer p-tyramine.

FIG. 17 shows dopamine dehydroxylation is inducible in E. lenta A2. E. lenta A2 was grown anaerobically either without or with 500 μM dopamine in BHI medium containing 1% (w/v) arginine and 10 mM formate. Cultures were spun down at OD600=0.500, and cell pellets were washed twice with anaerobic, sterile PBS to remove dopamine and any m-tyramine that had accumulated in the culture. Cells were lysed in 50 mM Tris pH 8 followed by addition of 250 μM dopamine to the lysate. Lysates were incubated at room temperature for 12 hours and were analyzed for m-tyramine production by LC-MS/MS. Lysate assays were performed anaerobically unless otherwise indicated.

FIG. 18 has two panels, A and B, and shows tungstate (WO42-) or molybdate (MoO42-) can be incorporated into the molybdenum dinucleotide cofactor during its complex biosynthesis, producing a cofactor where M is either W (tungsten) or Mo (molybdenum) (Panel A). This cofactor is then installed intact into its cognate enzyme, such as the dopamine dehydroxylase (Dadh). Panel B shows that the incorporation of tungsten into the cofactor will may prevent dopamine dehydroxylation by Dadh, while molybdenum should not impact dehydroxylation.

FIG. 19 shows anaerobic growth of E. lenta A2 in the presence of tungstate. E. lenta was grown anaerobically in BHI medium at 37° C. with varying concentrations of tungstate. There was no obvious dose-dependent growth defect of E. lenta in the presence of tungstate. The data shown are the mean of three replicate growth experiments±the SEM.

FIG. 20 shows E. lenta A2 lysate assays with tungstate. E. lenta A2 was grown either without or with 500 μM dopamine in BHI medium containing 1% (w/v) arginine and 10 mM formate. Cultures were spun down at OD600=0.500 and cell pellets were washed twice with anaerobic, sterile PBS to remove dopamine and any m-tyramine that had accumulated in the culture. Pellets were lysed anaerobically in 50 mM Tris pH 8 followed by addition of 500 μM dopamine (DA) to lysates. Lysates were left at room temperature for 12 hours and were analyzed for m-tyramine production by LC-MS/MS. Peak heights show the relative intensity of each mass and all chromatograms are shown on the same scale. There was no dose-dependent effect of tungstate on dopamine dehydroxylation, while oxygen (O2) completely blocked this activity.

FIG. 21 shows SDS-page of size exclusion chromatography fractions from activity-based purification of the dopamine dehydroxylase from E. lenta A2. Ladder is the Precision Plus Protein™ All Blue Standards (first lane from the left), while the subsequent lanes represent fractions from the size exclusion column, the last chromatography step of the activity-based purification from E. lenta A2. Each fraction was incubated for 12-14 hours anaerobically with 500 μM dopamine, 1 mM sodium dithionite, and 2 mM each of the electron donors benzyl viologen, methyl viologen, and diquat dibromide in 50 mM Tris pH 8 buffer at room temperature. After incubation, enzyme assay mixtures were analyzed by LC-MS/MS for dopamine and m-tyramine. The bar graphs represent the total dehydroxylation by each fraction (lanes 1-5), and this value was calculated as the concentration of m-tyramine normalized by the total concentration of m-tyramine and dopamine. The red asterisk indicates the band representing the dopamine dehydroxylase as confirmed by proteomics. This band tracks with activity, unlike other protein contaminants. The fraction showed in lane 5 was used for global proteomics, and the gel band representing the dopamine dehydroxylase (red asterisk) across lanes 4 and 5 was also cut out and subjected to proteomics to confirm this band's identity individually.

FIG. 22 shows global alignment of the dopamine dehydroxylase locus across an Actinobacterial library. Reads from genome sequencing were mapped to the reference E. lenta A2 genome contig containing the dopamine dehydroxylase and surrounding sequences using Bowtie2 and filtered for a minimum mapping quality of 10. Variants were called when >80% of reads supported an alternate sequence. Indel=insertion or deletion. Subst=amino acid substitution relative to the E. lenta A2 reference. Log2(FC)=log2fold change in gene expression in response to 500 μM dopamine relative to a vehicle control in E. lenta A2. El=Eggerthella lenta, Es=Eggerthella sinensis and Ph=Paraeggerthella hongonensis.

FIG. 23 shows alignment of the dopamine dehydroxylase protein across an Actinobacterial library. Sequences were retrieved by performing a pBLAST search of the E. lenta A2 dopamine dehydroxylase against a custom database of the Actinobacterial genomes in our collection. Alignment of the dopamine dehydroxylase sequences was performed in Jalview version 2.10.4, allowing for identification of the amino acid residue at position 506. Positions 493 through 511 are shown. Red asterisks indicate the positions that may explain metabolizer status.

FIG. 24 shows abundance of E. lenta in stool samples incubated with d₃-phenyl -L-dopa. Data represent qPCR with E. lenta-specific 16S rRNA primers. Each data point is the average abundance across 3 biological replicates for each sample shown in FIG. 3B. Bars represent the mean and standard error.

FIG. 25 shows correlation between E. faecalis and tyrDC abundance in fecal samples. Data represent qPCR with E. faecalis or tyrDC-specific primers. Each data point is the average abundance across 3 biological replicates for each sample shown in FIG. 3B. Linear regression indicated a highly significant correlation (R² =0.99, p<0.0001), suggesting E. faecalis is likely responsible for decarboxylation in these samples.

FIG. 26 has two panels, A and B, and shows metagenomic analysis of E. lenta/dadh and E. faecalis/tyrDC abundance and prevalence across human patients. Panel A shows a correlation of gene/bacterial abundances clearly demonstrates strong linear correlations between E. lenta/dadh and Enterococcus/tyrDC (R²>0.812, P<2.2e−16 Pearson's Correlation). Panel B shows a prevalence estimates as a function of minimum abundance reveals that both dadh and tyrDC are highly prevalent in the human gut microbiome albeit at low relative abundances.

FIG. 27 shows gain of function studies in stool samples incubated with d₃-phenyl -L-dopa. A subset of non-metabolizing samples from FIG. 3, Panel B were incubated anaerobically in MEGA medium containing d₃-phenyl-L-dopa (1 mM) for 72 hours at 37° C. LC-MS/MS was used to quantify metabolites. The samples were incubated either without any additional strains, or with E. faecalis MMH594 wild-type, E. faecalis tyrDC mutant, or E. lenta A2. Each panel represents one individual. Bars represent the mean±standard error.

FIG. 28 shows abundance of E. lenta in stool samples incubated with dopamine. Data represent qPCR with E. lenta-specific 16S rRNA primers. Each data point is the average abundance across 3 biological replicates for each sample shown in FIG. 3, Panel B. Bars represent the mean and standard error. Each data point is the E. lenta abundance in each sample shown in FIG. 3, Panel E.

FIG. 29 shows SNP analysis of dadh in human gut metagenomes containing high-coverage E. lenta genomes. The two amino acid variants of interest (FIG. 18): p.Ser500Cys p.Arg506Ser were found to be supported in metagenomes with the dominant SNP prevalence similar to those observed in the isolate strain collection (1/76 and 38/77 respectively).

FIG. 30 has four panels, A-D, and shows that E. faecalis and tyrDC predict L-dopa decarboxylation and dehydroxylation in complex human gut microbiotas from Parkinson's disease patients. Panel A shows LC-MS/MS was used to quantify metabolism of d₃-phenyl-L-dopa by 12 unrelated human gut microbiota samples from Parkinson's disease patients ex vivo. Samples were cultured anaerobically in MEGA medium containing d₃-phenyl-L-dopa (0.5 mM) for 72 hours. Metabolite levels in culture supernatants were analyzed by LC-MS/MS. Stacked bar plots represent the mean concentration±the SEM of three biological replicates. Samples 1-6 represent patients not currently taking L-dopa/carbidopa, while samples 7-12 are from patients taking L-dopa/carbidopa at the time of collection (Panel B) LC-MS/MS was used to quantify phenolic acid metabolites of d₃-phenyl-L-dopa in patients showing detectable L-dopa depletion without production of dopamine or m-tyramine in panel (A). Panel C shows the abundance of E. faecalis predicts L-dopa decarboxylation in human gut microbiota samples from Parkinson's disease patients. Data represent qPCR with E. faecalis-specific 16S rRNA primers. Each data point is the average abundance across 3 biological replicates for each sample shown in (A) **P=0.0040, one tailed Mann-Whitney test. Panel D whos the abundance of tyrDC predicts L-dopa decarboxylation in human gut microbiota samples from Parkinson's disease patients. Data represent qPCR with tyrDC-specific primers. Each data point is the average abundance across 3 biological replicates for each sample shown in (Panel B). Bars represent the mean and standard error. **P=0.0020, one tailed Mann-Whitney test.

FIG. 31 has four panels, A-D, and shows that E. lenta Dadh predict L-dopa metabolism in complex human gut microbiotas from Parkinson's disease patients. Panel A LC-MS was used to quantify dopamine dehydroxylation in the fecal microbiotas of 12 unrelated Parkinson's disease patients. Samples were cultured for 48 hours in BHI medium containing 1% (w/v) arginine, 10 mM formate, and 0.5 mM dopamine and metabolites were analyzed by LC-MS. (A) Bars represent the mean and SEM of n=3 for low reducers (<50%) and n=9 for high reducers (>50%) **P=0.0045, one tailed Mann-Whitney test, low reducers vs. high reducers. Panel B Dadh abundance does not correlate with dehydroxylation by fecal microbiomes from 12 unrelated Parkinson's disease patients. Data represent qPCR with Dadh-specific primers. Each data point is the dadh abundance in each sample shown in Panel A. Bars represent the mean and standard error. Panel C shows E. lenta abundance does not correlate with dehydroxylation by fecal microbiotas from 12 unrelated Parkinson's patients. Data represent qPCR with E. lenta-specific primers. Each data point is the E. lenta abundance in each sample shown in Panel A. Bars represent the mean and standard error. Panel D shows Dadh sequence variants predict dopamine dehydroxylation ex vivo. gDNA from each culture in Panel A was extracted followed by PCR amplification of the dadh gene. The identities of the sequence variants were assessed by Sanger sequencing of dadh using primers specific for the region containing position 506. Samples where a mix of variants were present (n=6) were removed. Bars represent the mean and SEM (n=2 for AGC samples, n=4 for CGC samples). While all AGC samples were low metabolizers (<30%) and all CGC samples displayed quantitative metabolism (>98%), the difference in dehydroxylation between the groups did not reach statistical significance, likely due to the small number of samples involved (p=0.067, one-tailed Mann-Whitney test, n=2 AGC vs n=4 CGC).

FIG. 32 has two panels, A and B, and shows anaerobic growth of E. faecalis MMH594 with carbidopa or AFMT. E. faecalis was grown anaerobically in BHI medium at 37° C. with varying concentrations of (Panel A) carbidopa or (Panel B) AFMT. There was no obvious dose-dependent growth defect of E. faecalis in the presence of these inhibitors. The data shown are the mean of three replicate growth experiments±the SEM.

FIG. 33 has two panels, A and B, and shows anaerobic growth of E. lenta A2 with carbidopa or AFMT. E. lenta was grown anaerobically in BHI medium at 37° C. with varying concentrations of (Panel A) carbidopa or (Panel B) AFMT. There was no obvious dose-dependent growth defect of E. lenta in the presence of these inhibitors. The data shown is the mean of three replicate growth experiments±the SEM.

FIG. 34 has two panels, A and B, and shows dehydroxylation of dopamine by E. lenta A2 in the presence of carbidopa or AFMT. E. lenta A2 was grown anaerobically in BHI medium at 37° C. for 48 hours with 500 μM dopamine and varying concentrations of (Panel A) carbidopa or (Panel B) AFMT. Culture supernatants were analyzed for dopamine and m-tyramine by LC-MS/MS. % Dopamine dehydroxylation was calculated as the concentration of m-tyramine relative to the total concentration of dopamine and m-tyramine. There was no obvious dose-dependent defect in dopamine dehydroxylation E. lenta A2 in the presence of these inhibitors. The data shown is the mean of three replicate experiments±the SEM.

FIG. 35 shows time course of degradation of d₃-phenyl-L-dopa in the presence and absence of 2 mM carbidopa in a stool sample from a neurologically healthy patient. A metabolizing sample from FIG. 3, Panel B was incubated in MEGA medium containing d₃-phenyl-L-dopa (1 mM) with or without carbidopa (2 mM) for 72 hours. LC-MS/MS was used to quantify metabolites at 12, 24, 48, and 72 hours. The data shown are the mean of three replicate experiments±the SEM.

FIG. 36 has three panels, A-C, and shows activity of carbidopa in human fecal microbiota samples. Fecal samples were incubated anaerobically in MEGA medium containing 0 or 2 mM carbidopa and 1 mM d₃-phenyl-L-dopa for 72 hours at 37° C. Metabolites in culture supernatants were analyzed by LC-MS/MS. Error bars represent the mean±the SEM for three biological replicates. Each panel represents a different individual. Panel A) is from a neurologically healthy patient, while Panel B) and Panel C) are from Parkinson's disease patients.

FIG. 37 shows a screen of amino acid substrates for AADC and TyrDC. AADC (0.20 μM) or TyrDC (0.15 μM) were incubated with varying substrates (500 μM of L-dopa, phenylalanine, p-tyrosine, or m-tyrosine) at room temperature (0.1 M pH 7.4 phosphate buffer for AADC, 0.2 M pH 5.5 sodium acetate buffer for TyrDC) and reaction rates were measured by LC-MS/MS. The AADC reaction was quenched at 180 seconds and the TyrDC reaction was quenched at 60 seconds by dissolving 1:10 in methanol. Reactions were performed at room temperature. Reaction supernatants were analyzed for production of the primary amines resulting from decarboxylation by LC-MS/MS. % Activity represents the rate relative to the most rapidly consumed substrate (m-tyrosine for AADC, p-tyrosine for TyrDC). The data shown is the mean of three replicate experiments±the SEM.

FIG. 38 shows metabolism of L-dopa by co-cultures of E. faecalis and E. lenta strains in the presence of AFMT. Strains were co-cultured for 48 hours in BHI medium containing 0.5% arginine and 1 mM d₃-phenyl-L-dopa with or without 125 μM AFMT. Metabolites in culture supernatants were analyzed by LC-MS/MS. Stacked bar plots represent the mean±the SEM of three biological replicates.

FIG. 39 has three panels, A-C, and shows activity of AFMT in human fecal microbiota samples. Fecal samples were incubated anaerobically in MEGA medium containing 0 or 2 mM carbidopa and 1 mM d₃-phenyl-L-dopa for 72 hours at 37° C. Metabolites in culture supernatants were analyzed by LC-MS/MS. Error bars represent the mean±the SEM for three biological replicates. Each panel represents a different individual. Panel A) and Panel B) are from neurologically healthy patients, Panel C) is from a Parkinson's disease patient.

FIG. 40 shows MTS assay evaluating AMFT toxicity toward HeLa cells. HeLa cells were seeded in 100 μL growth medium [(DMEM medium supplemented with 10% FBS (2 mL) and 1× Antibiotic-Antimycotic (100× stock, Invitrogen))] and incubated at 37° C. in 5% CO₂ incubator for 1 day. Wells containing growth medium only were used as background controls. Cells were treated with various concentrations of AFMT in quadruplicate. Two days post treatment, 20 μL of CellTiter 96® AQueous One Solution Reagent (Promega) was added to each well. The plates were incubated at 37° C. in 5% CO₂ incubator for 2 hours followed by absorbance measurement at 490 nm. To calculate relative cell viability, the readings for each compound concentration were subtracted from the background controls and normalized to vehicle controls.

### DETAILED DESCRIPTION

### General

The human gut microbiota metabolizes the Parkinson's disease medication Levodopa (L-dopa), potentially reducing drug availability and causing side effects. However, the organisms, genes, and enzymes responsible for this activity in patients and their susceptibility to inhibition by host-targeted drugs are unknown. Here, an interspecies pathway for gut bacterial L-dopa metabolism is described. Conversion of L-dopa to dopamine by a pyridoxal phosphate-dependent tyrosine decarboxylase from Enterococcus faecalis is followed by transformation of dopamine to m-tyramine by a molybdenum-dependent dehydroxylase from Eggerthella lenta. These enzymes predict drug metabolism in complex human gut microbiotas. While a drug targeting host aromatic amino acid decarboxylase does not prevent gut microbial L-dopa decarboxylation, herein a compound is described that inhibits this activity in Parkinson's patient microbiotas and increases L-dopa bioavailability in mice.

Provided herein are methods and compositions related to treating a condition in a subject comprising administering an agent that inhibits the activity of or decreases the levels of L-dopa decarboxylase conjointly with levodopa (L-dopa). Also provided herein are methods of treating Parkinson's Disease in a subject and/or method of treating or preventing symptoms resulting from dopaminergic neuron loss in a subject in need thereof by administering an agent to the subject that inhibits the activity of or decreases the levels of TyrDC conjointly with levodopa. Also provided herein are in-vitro methods of determining whether an agent is a therapeutic agent for Parkinson's Disease comprising determining whether the test agent decreases the levels of or inhibits the activity of a TyrDC enzyme, wherein the test agent is determined to be a therapeutic agent for the treatment of Parkinson's Disease if the test agent decreases the levels of or inhibits the activity of a TyrDC enzyme. The test agent may be a member of a library of test agents. The test agent may be an interfering nucleic acid, a peptide, a small molecule, or an antibody. The agent may decrease the level of or inhibits the activity of the TyrDC enzyme by at least 10%, at least 20%, at least 30%, at least 40%, at least 50%, at least 75%, or at least 95%.

Provided herein are methods of treating a condition in a subject, comprising administering a composition that inhibits the activity of or decreases the levels of a bacteria that expresses a PLP-dependent tyrosine decarboxylase (TyrDC) or a TyrDC homolog conjointly with levodopa.

### Definitions

For convenience, certain terms employed in the specification, examples, and appended claims are collected here.

As used herein, the term “administering” means providing a pharmaceutical agent or composition to a subject, and includes, but is not limited to, administering by a medical professional and self-administering.

The term “agent” is used herein to denote a chemical compound, a small molecule, a mixture of chemical compounds and/or a biological macromolecule (such as a nucleic acid, an antibody, an antibody fragment, a protein or a peptide). Agents may be identified as having a particular activity by screening assays described herein below. The activity of such agents may render them suitable as a “therapeutic agent” which is a biologically, physiologically, or pharmacologically active substance (or substances) that acts locally or systemically in a subject.

The term “amino acid” is intended to embrace all molecules, whether natural or synthetic, which include both an amino functionality and an acid functionality and capable of being included in a polymer of naturally occurring amino acids. Exemplary amino acids include naturally occurring amino acids; analogs, derivatives and congeners thereof; amino acid analogs having variant side chains; and all stereoisomers of any of the foregoing.

As used herein, the term “antibody” may refer to both an intact antibody and an antigen binding fragment thereof. Intact antibodies are glycoproteins that include at least two heavy (H) chains and two light (L) chains inter-connected by disulfide bonds. Each heavy chain includes a heavy chain variable region (abbreviated herein as V_{H}) and a heavy chain constant region. Each light chain includes a light chain variable region (abbreviated herein as V_{L}) and a light chain constant region. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system. The term “antibody” includes, for example, monoclonal antibodies, polyclonal antibodies, chimeric antibodies, humanized antibodies, human antibodies, multispecific antibodies (e.g., bispecific antibodies), single-chain antibodies and antigen-binding antibody fragments. An “isolated antibody,” as used herein, refers to an antibody which is substantially free of other antibodies having different antigenic specificities. An isolated antibody may, however, have some cross-reactivity to other, related antigens.

The terms “antigen binding fragment” and “antigen-binding portion” of an antibody, as used herein, refers to one or more fragments of an antibody that retain the ability to bind to an antigen. Examples of binding fragments encompassed within the term “antigen-binding fragment” of an antibody include Fab, Fab′, F(ab′)₂, Fv, scFv, disulfide linked Fv, Fd, diabodies, single-chain antibodies, NANOBODIES®, isolated CDRH3, and other antibody fragments that retain at least a portion of the variable region of an intact antibody. These antibody fragments can be obtained using conventional recombinant and/or enzymatic techniques and can be screened for antigen binding in the same manner as intact antibodies.

The terms “CDR”, and its plural “CDRs”, refer to a complementarity determining region (CDR) of an antibody or antibody fragment, which determine the binding character of an antibody or antibody fragment. In most instances, three CDRs are present in a light chain variable region (CDRL1, CDRL2 and CDRL3) and three CDRs are present in a heavy chain variable region (CDRH1, CDRH2 and CDRH3). CDRs contribute to the functional activity of an antibody molecule and are separated by amino acid sequences that comprise scaffolding or framework regions. Among the various CDRs, the CDR3 sequences, and particularly CDRH3, are the most diverse and therefore have the strongest contribution to antibody specificity. There are at least two techniques for determining CDRs: (1) an approach based on cross-species sequence variability (i.e., Kabat et al., Sequences of Proteins of Immunological Interest (National Institute of Health, Bethesda, Md. (1987), incorporated by reference in its entirety); and (2) an approach based on crystallographic studies of antigen-antibody complexes (Chothia et al., Nature, 342:877 (1989), incorporated by reference in its entirety).

As used herein, the term “humanized antibody” refers to an antibody that has at least one CDR derived from a mammal other than a human, and a FR region and the constant region of a human antibody. A humanized antibody is useful as an effective component in a therapeutic agent since antigenicity of the humanized antibody in human body is lowered.

The term “isolated polypeptide” refers to a polypeptide, in certain embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated nucleic acid” refers to a polynucleotide of genomic, cDNA, or synthetic origin or some combination thereof, which (1) is not associated with the cell in which the “isolated nucleic acid” is found in nature, or (2) is operably linked to a polynucleotide to which it is not linked in nature.

As used herein, the term “monoclonal antibody” refers to an antibody obtained from a population of substantially homogeneous antibodies that specifically bind to the same epitope, i.e., the individual antibodies comprising the population are identical except for possible naturally occurring mutations that may be present in minor amounts. The modifier “monoclonal” indicates the character of the antibody as being obtained from a substantially homogeneous population of antibodies, and is not to be construed as requiring production of the antibody by any particular method.

The terms “polynucleotide”, and “nucleic acid” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified, such as by conjugation with a labeling component. The term “recombinant” polynucleotide means a polynucleotide of genomic, cDNA, semisynthetic, or synthetic origin which either does not occur in nature or is linked to another polynucleotide in a non-natural arrangement.

The phrase “pharmaceutically-acceptable carrier” as used herein means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body

As used herein, “specific binding” refers to the ability of an antibody to bind to a predetermined antigen or the ability of a polypeptide to bind to its predetermined binding partner. Typically, an antibody or polypeptide specifically binds to its predetermined antigen or binding partner with an affinity corresponding to a K_{D} of about 10⁻⁷ M or less, and binds to the predetermined antigen/binding partner with an affinity (as expressed by K_{D}) that is at least 10 fold less, at least 100 fold less or at least 1000 fold less than its affinity for binding to a non-specific and unrelated antigen/binding partner (e.g., BSA, casein).

As used herein, the term “subject” means a human or non-human animal selected for treatment or therapy.

The phrases “therapeutically-effective amount” and “effective amount” as used herein means the amount of an agent which is effective for producing the desired therapeutic effect in at least a sub-population of cells in a subject at a reasonable benefit/risk ratio applicable to any medical treatment. “Treating” a disease in a subject or “treating” a subject having a disease refers to subjecting the subject to a pharmaceutical treatment, e.g., the administration of a drug, such that at least one symptom of the disease is decreased or prevented from worsening.

### Small Molecule Agents

Certain embodiments disclosed herein relate to agents and methods for treating or preventing a condition (e.g., any condition, disease, disorder, or indication disclosed herein) in a subject comprising administering an agent that inhibits the activity of or decreases the levels of L-dopa decarboxylase conjointly with levodopa (L-dopa). Also provided herein are methods of treating Parkinson's Disease in a subject and/or method of treating or preventing symptoms resulting from dopaminergic neuron loss in a subject in need thereof by administering an agent to the subject that inhibits the activity of or decreases the levels of TyrDC conjointly with levodopa. In some embodiments, the agent preferentially inhibits the activity of or decreases the level of TyrDC over amino acid decarboxylase (AADC).

The agent may be a small molecule (e.g., (S)-α-Fluoromethyltyrosine (AFMT)). Additionally, the agents disclosed herein are used in methods of treating Parkinson's Disease in a subject and/or methods of treating or preventing symptoms resulting from dopaminergic neuron loss in a subject in need thereof by administering an agent to the subject that inhibits the activity of or decreases the levels of TyrDC conjointly with levodopa. Such agents include those disclosed herein, those known in the art and those identified using the screening assays described herein. For example, in some embodiments the agent inhibits the activity of or decreases the levels of L-dopa decarboxylase. Examples of inhibitors of L-dopa decarboxylase include, but are not limited to, AFMT, or pharmaceutically acceptable salts thereof.

Agents useful in the methods disclosed herein may be obtained from any available source, including systematic libraries of natural and/or synthetic compounds. Agents may also be obtained by any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive; see, e.g., Zuckermann et al., 1994, J. Med. Chem. 37:2678-85); spatially addressable parallel solid phase or solution phase libraries; synthetic library methods requiring deconvolution; the ‘one-bead one-compound’ library method; and synthetic library methods using affinity chromatography selection. The biological library and peptoid library approaches are limited to peptide libraries, while the other four approaches are applicable to peptide, non-peptide oligomer or small molecule libraries of compounds (Lam, 1997, Anticancer Drug Des. 12:145).

Examples of methods for the synthesis of molecular libraries can be found in the art, for example in: DeWitt et al. (1993) Proc. Natl. Acad. Sci. U.S.A. 90:6909; Erb et al. (1994) Proc. Natl. Acad. Sci. USA 91:11422; Zuckermann et al. (1994). J. Med. Chem. 37:2678; Cho et al. (1993) Science 261:1303; Carrell et al. (1994) Angew. Chem. Int. Ed. Engl. 33:2059; Carell et al. (1994) Angew. Chem. Int. Ed. Engl. 33:2061; and in Gallop et al. (1994) J. Med. Chem. 37:1233.

Libraries of agents may be presented in solution (e.g., Houghten, 1992, Biotechniques 13:412-421), or on beads (Lam, 1991, Nature 354:82-84), chips (Fodor, 1993, Nature 364:555-556), bacteria and/or spores, (Ladner, U.S. Pat. No. 5,223,409), plasmids (Cull et al, 1992, Proc Natl Acad Sci USA 89:1865-1869) or on phage (Scott and Smith, 1990, Science 249:386-390; Devlin, 1990, Science 249:404-406; Cwirla et al, 1990, Proc. Natl. Acad. Sci. 87:6378-6382; Felici, 1991, J. Mol. Biol. 222:301-310; Ladner, supra.).

### Interfering Nucleic Acid Agents

In certain embodiments, interfering nucleic acid molecules that selectively target a product of a gene that encodes for an L-dopa decarboxylase are provided herein. Interfering nucleic acids generally include a sequence of cyclic subunits, each bearing a base-pairing moiety, linked by intersubunit linkages that allow the base-pairing moieties to hybridize to a target sequence in a nucleic acid (typically an RNA) by Watson-Crick base pairing, to form a nucleic acid:oligomer heteroduplex within the target sequence. Interfering RNA molecules include, but are not limited to, antisense molecules, siRNA molecules, single-stranded siRNA molecules, miRNA molecules and shRNA molecules.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acid molecule is double-stranded RNA. The double-stranded RNA molecule may have a 2 nucleotide 3′ overhang. In some embodiments, the two RNA strands are connected via a hairpin structure, forming a shRNA molecule. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

Interfering nucleic acid molecules provided herein can contain RNA bases, non-RNA bases or a mixture of RNA bases and non-RNA bases. For example, interfering nucleic acid molecules provided herein can be primarily composed of RNA bases but also contain DNA bases or non-naturally occurring nucleotides.

The interfering nucleic acids can employ a variety of oligonucleotide chemistries. Examples of oligonucleotide chemistries include, without limitation, peptide nucleic acid (PNA), linked nucleic acid (LNA), phosphorothioate, 2′O-Me-modified oligonucleotides, and morpholino chemistries, including combinations of any of the foregoing. In general, PNA and LNA chemistries can utilize shorter targeting sequences because of their relatively high target binding strength relative to 2′O-Me oligonucleotides. Phosphorothioate and 2′O-Me-modified chemistries are often combined to generate 2′O-Me-modified oligonucleotides having a phosphorothioate backbone. See, e.g., PCT Publication Nos. WO/2013/112053 and WO/2009/008725, incorporated by reference in their entireties.

Peptide nucleic acids (PNAs) are analogs of DNA in which the backbone is structurally homomorphous with a deoxyribose backbone, consisting of N-(2-aminoethyl) glycine units to which pyrimidine or purine bases are attached. PNAs containing natural pyrimidine and purine bases hybridize to complementary oligonucleotides obeying Watson-Crick base-pairing rules, and mimic DNA in terms of base pair recognition (Egholm, Buchardt et al. 1993). The backbone of PNAs is formed by peptide bonds rather than phosphodiester bonds, making them well-suited for antisense applications (see structure below). The backbone is uncharged, resulting in PNA/DNA or PNA/RNA duplexes that exhibit greater than normal thermal stability. PNAs are not recognized by nucleases or proteases.

Despite a radical structural change to the natural structure, PNAs are capable of sequence-specific binding in a helix form to DNA or RNA. Characteristics of PNAs include a high binding affinity to complementary DNA or RNA, a destabilizing effect caused by single-base mismatch, resistance to nucleases and proteases, hybridization with DNA or RNA independent of salt concentration and triplex formation with homopurine DNA. PANAGENE™. has developed its proprietary Bts PNA monomers (Bts; benzothiazole-2-sulfonyl group) and proprietary oligomerization process. The PNA oligomerization using Bts PNA monomers is composed of repetitive cycles of deprotection, coupling and capping. PNAs can be produced synthetically using any technique known in the art. See, e.g., U.S. Pat. Nos. 6,969,766, 7,211,668, 7,022,851, 7,125,994, 7,145,006 and 7,179,896. See also U.S. Pat. Nos. 5,539,082; 5,714,331; and 5,719,262 for the preparation of PNAs. Further teaching of PNA compounds can be found in Nielsen et al., Science, 254:1497-1500, 1991. Each of the foregoing is incorporated by reference in its entirety.

Interfering nucleic acids may also contain “locked nucleic acid” subunits (LNAs). “LNAs” are a member of a class of modifications called bridged nucleic acid (BNA). BNA is characterized by a covalent linkage that locks the conformation of the ribose ring in a C30-endo (northern) sugar pucker. For LNA, the bridge is composed of a methylene between the 2′-O and the 4′-C positions. LNA enhances backbone preorganization and base stacking to increase hybridization and thermal stability.

The structures of LNAs can be found, for example, in Wengel, et al., Chemical Communications (1998) 455; Tetrahedron (1998) 54:3607, and Accounts of Chem. Research (1999) 32:301); Obika, et al., Tetrahedron Letters (1997) 38:8735; (1998) 39:5401, and Bioorganic Medicinal Chemistry (2008) 16:9230. Compounds provided herein may incorporate one or more LNAs; in some cases, the compounds may be entirely composed of LNAs. Methods for the synthesis of individual LNA nucleoside subunits and their incorporation into oligonucleotides are described, for example, in U.S. Pat. Nos. 7,572,582, 7,569,575, 7,084,125, 7,060,809, 7,053,207, 7,034,133, 6,794,499, and 6,670,461, each of which is incorporated by reference in its entirety. Typical intersubunit linkers include phosphodiester and phosphorothioate moieties; alternatively, non-phosphorous containing linkers may be employed. One embodiment is an LNA containing compound where each LNA subunit is separated by a DNA subunit. Certain compounds are composed of alternating LNA and DNA subunits where the intersubunit linker is phosphorothioate.

“Phosphorothioates” (or S-oligos) are a variant of normal DNA in which one of the nonbridging oxygens is replaced by a sulfur. The sulfurization of the internucleotide bond reduces the action of endo-and exonucleases including 5′ to 3′ and 3′ to 5′ DNA POL 1 exonuclease, nucleases S1 and P1, RNases, serum nucleases and snake venom phosphodiesterase. Phosphorothioates are made by two principal routes: by the action of a solution of elemental sulfur in carbon disulfide on a hydrogen phosphonate, or by the method of sulfurizing phosphite triesters with either tetraethylthiuram disulfide (TETD) or 3H-1, 2-bensodithiol-3-one 1, 1-dioxide (BDTD) (see, e.g., Iyer et al., J. Org. Chem. 55, 4693-4699, 1990). The latter methods avoid the problem of elemental sulfur's insolubility in most organic solvents and the toxicity of carbon disulfide. The TETD and BDTD methods also yield higher purity phosphorothioates.

“2′O-Me oligonucleotides” molecules carry a methyl group at the 2′-OH residue of the ribose molecule. 2′-O-Me-RNAs show the same (or similar) behavior as DNA, but are protected against nuclease degradation. 2′-O-Me-RNAs can also be combined with phosphothioate oligonucleotides (PTOs) for further stabilization. 2′O-Me oligonucleotides (phosphodiester or phosphothioate) can be synthesized according to routine techniques in the art (see, e.g., Yoo et al., Nucleic Acids Res. 32:2008-16, 2004).

The interfering nucleic acids described herein may be contacted with a cell or administered to an organism (e.g., a human). Alternatively, constructs and/or vectors encoding the interfering RNA molecules may be contacted with or introduced into a cell or organism. In certain embodiments, a viral, retroviral or lentiviral vector is used. In some embodiments, the vector has a tropism for cardiac tissue. In some embodiments the vector is an adeno-associated virus.

Typically at least 17, 18, 19, 20, 21, 22 or 23 nucleotides of the complement of the target mRNA sequence are sufficient to mediate inhibition of a target transcript. Perfect complementarity is not necessary. In some embodiments, the interfering nucleic acids contains a 1, 2 or 3 nucleotide mismatch with the target sequence. The interfering nucleic acid molecule may have a 2 nucleotide 3′ overhang. If the interfering nucleic acid molecule is expressed in a cell from a construct, for example from a hairpin molecule or from an inverted repeat of the desired sequence, then the endogenous cellular machinery will create the overhangs. shRNA molecules can contain hairpins derived from microRNA molecules. For example, an RNAi vector can be constructed by cloning the interfering RNA sequence into a pCAG-miR30 construct containing the hairpin from the miR30 miRNA. RNA interference molecules may include DNA residues, as well as RNA residues.

In some embodiments, the interfering nucleic acid molecule is a siRNA molecule. Such siRNA molecules should include a region of sufficient homology to the target region, and be of sufficient length in terms of nucleotides, such that the siRNA molecule down-regulate target RNA. The term “ribonucleotide” or “nucleotide” can, in the case of a modified RNA or nucleotide surrogate, also refer to a modified nucleotide, or surrogate replacement moiety at one or more positions. It is not necessary that there be perfect complementarity between the siRNA molecule and the target, but the correspondence must be sufficient to enable the siRNA molecule to direct sequence-specific silencing, such as by RNAi cleavage of the target RNA. In some embodiments, the sense strand need only be sufficiently complementary with the antisense strand to maintain the overall double-strand character of the molecule.

In addition, an siRNA molecule may be modified or include nucleoside surrogates. Single stranded regions of an siRNA molecule may be modified or include nucleoside surrogates, e.g., the unpaired region or regions of a hairpin structure, e.g., a region which links two complementary regions, can have modifications or nucleoside surrogates. Modification to stabilize one or more 3′- or 5′-terminus of an siRNA molecule, e.g., against exonucleases, or to favor the antisense siRNA agent to enter into RISC are also useful. Modifications can include C3 (or C6, C7, C12) amino linkers, thiol linkers, carboxyl linkers, non-nucleotidic spacers (C3, C6, C9, C12, abasic, triethylene glycol, hexaethylene glycol), special biotin or fluorescein reagents that come as phosphoramidites and that have another DMT-protected hydroxyl group, allowing multiple couplings during RNA synthesis.

Each strand of an siRNA molecule can be equal to or less than 35, 30, 25, 24, 23, 22, 21, or 20 nucleotides in length. In some embodiments, the strand is at least 19 nucleotides in length. For example, each strand can be between 21 and 25 nucleotides in length. In some embodiments, siRNA agents have a duplex region of 17, 18, 19, 29, 21, 22, 23, 24, or 25 nucleotide pairs, and one or more overhangs, such as one or two 3′ overhangs, of 2-3 nucleotides.

A “small hairpin RNA” or “short hairpin RNA” or “shRNA” includes a short RNA sequence that makes a tight hairpin turn that can be used to silence gene expression via RNA interference. The shRNAs provided herein may be chemically synthesized or transcribed from a transcriptional cassette in a DNA plasmid. The shRNA hairpin structure is cleaved by the cellular machinery into siRNA, which is then bound to the RNA-induced silencing complex (RISC).

In some embodiments, shRNAs are about 15-60, 15-50, or 15-40 (duplex) nucleotides in length, about 15-30, 15-25, or 19-25 (duplex) nucleotides in length, or are about 20-24, 21-22, or 21-23 (duplex) nucleotides in length (e.g., each complementary sequence of the double-stranded shRNA is 15-60, 15-50, 15-40, 15-30, 15-25, or 19-25 nucleotides in length, or about 20-24, 21-22, or 21-23 nucleotides in length, and the double-stranded shRNA is about 15-60, 15-50, 15-40, 15-30, 15-25, or 19-25 base pairs in length, or about 18-22, 19-20, or 19-21 base pairs in length). shRNA duplexes may comprise 3′ overhangs of about 1 to about 4 nucleotides or about 2 to about 3 nucleotides on the antisense strand and/or 5′-phosphate termini on the sense strand. In some embodiments, the shRNA comprises a sense strand and/or antisense strand sequence of from about 15 to about 60 nucleotides in length (e.g., about 15-60, 15-55, 15-50, 15-45, 15-40, 15-35, 15-30, or 15-25 nucleotides in length),or from about 19 to about 40 nucleotides in length (e.g., about 19-40, 19-35, 19-30, or 19-25 nucleotides in length), or from about 19 to about 23 nucleotides in length (e.g., 19, 20, 21, 22, or 23 nucleotides in length).

Non-limiting examples of shRNA include a double-stranded polynucleotide molecule assembled from a single-stranded molecule, where the sense and antisense regions are linked by a nucleic acid-based or non-nucleic acid-based linker; and a double-stranded polynucleotide molecule with a hairpin secondary structure having self-complementary sense and antisense regions. In some embodiments, the sense and antisense strands of the shRNA are linked by a loop structure comprising from about 1 to about 25 nucleotides, from about 2 to about 20 nucleotides, from about 4 to about 15 nucleotides, from about 5 to about 12 nucleotides, or 1, 2, 3, 4, 5, 6, 7, 8, 9, 10, 11, 12, 13, 14, 15, 16, 17, 18, 19, 20, 21, 22, 23, 24, 25, or more nucleotides.

Additional embodiments related to the shRNAs, as well as methods of designing and synthesizing such shRNAs, are described in U.S. patent application publication number 2011/0071208, the disclosure of which is herein incorporated by reference in its entirety for all purposes.

In some embodiments, provided herein are micro RNAs (miRNAs). miRNAs represent a large group of small RNAs produced naturally in organisms, some of which regulate the expression of target genes. miRNAs are formed from an approximately 70 nucleotide single-stranded hairpin precursor transcript by Dicer. miRNAs are not translated into proteins, but instead bind to specific messenger RNAs, thereby blocking translation. In some instances, miRNAs base-pair imprecisely with their targets to inhibit translation.

In some embodiments, antisense oligonucleotide compounds are provided herein. In certain embodiments, the degree of complementarity between the target sequence and antisense targeting sequence is sufficient to form a stable duplex. The region of complementarity of the antisense oligonucleotides with the target RNA sequence may be as short as 8-11 bases, but can be 12-15 bases or more, e.g., 10-40 bases, 12-30 bases, 12-25 bases, 15-25 bases, 12-20 bases, or 15-20 bases, including all integers in between these ranges. An antisense oligonucleotide of about 14-15 bases is generally long enough to have a unique complementary sequence.

In certain embodiments, antisense oligonucleotides may be 100% complementary to the target sequence, or may include mismatches, e.g., to improve selective targeting of allele containing the disease-associated mutation, as long as a heteroduplex formed between the oligonucleotide and target sequence is sufficiently stable to withstand the action of cellular nucleases and other modes of degradation which may occur in vivo. Hence, certain oligonucleotides may have about or at least about 70% sequence complementarity, e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence complementarity, between the oligonucleotide and the target sequence. Oligonucleotide backbones that are less susceptible to cleavage by nucleases are discussed herein. Mismatches, if present, are typically less destabilizing toward the end regions of the hybrid duplex than in the middle. The number of mismatches allowed will depend on the length of the oligonucleotide, the percentage of G:C base pairs in the duplex, and the position of the mismatch(es) in the duplex, according to well understood principles of duplex stability.

Interfering nucleic acid molecules can be prepared, for example, by chemical synthesis, in vitro transcription, or digestion of long dsRNA by Rnase III or Dicer. These can be introduced into cells by transfection, electroporation, or other methods known in the art. See Hannon, G J, 2002, RNA Interference, Nature 418: 244-251; Bernstein E et al., 2002, The rest is silence. RNA 7: 1509-1521; Hutvagner Get al., RNAi: Nature abhors a double-strand. Curr. Opin. Genetics & Development 12: 225-232; Brummelkamp, 2002, A system for stable expression of short interfering RNAs in mammalian cells. Science 296: 550-553; Lee NS, Dohjima T, Bauer G, Li H, Li M-J, Ehsani A, Salvaterra P, and Rossi J. (2002). Expression of small interfering RNAs targeted against HIV-1 rev transcripts in human cells. Nature Biotechnol. 20:500-505; Miyagishi M, and Taira K. (2002). U6-promoter-driven siRNAs with four uridine 3′ overhangs efficiently suppress targeted gene expression in mammalian cells. Nature Biotechnol. 20:497-500; Paddison P J, Caudy A A, Bernstein E, Hannon G J, and Conklin D S. (2002). Short hairpin RNAs (shRNAs) induce sequence-specific silencing in mammalian cells. Genes & Dev. 16:948-958; Paul C P, Good P D, Winer I, and Engelke D R. (2002). Effective expression of small interfering RNA in human cells. Nature Biotechnol. 20:505-508; Sui G, Soohoo C, Affar E-B, Gay F, Shi Y, Forrester W C, and Shi Y. (2002). A DNA vector-based RNAi technology to suppress gene expression in mammalian cells. Proc. Natl. Acad. Sci. USA 99(6):5515-5520; Yu J-Y, DeRuiter S L, and Turner D L. (2002). RNA interference by expression of short-interfering RNAs and hairpin RNAs in mammalian cells. Proc. Natl. Acad. Sci. USA 99(9):6047-6052.

In the present methods, an interfering nucleic acid molecule or an interfering nucleic acid encoding polynucleotide can be administered to the subject, for example, as naked nucleic acid, in combination with a delivery reagent, and/or as a nucleic acid comprising sequences that express an interfering nucleic acid molecule. In some embodiments the nucleic acid comprising sequences that express the interfering nucleic acid molecules are delivered within vectors, e.g. plasmid, viral and bacterial vectors. Any nucleic acid delivery method known in the art can be used in the methods described herein. Suitable delivery reagents include, but are not limited to, e.g., the Minis Transit TKO lipophilic reagent; lipofectin; lipofectamine; cellfectin; polycations (e.g., polylysine), atelocollagen, nanoplexes and liposomes. The use of atelocollagen as a delivery vehicle for nucleic acid molecules is described in Minakuchi et al. Nucleic Acids Res., 32(13):e109 (2004); Hanai et al. Ann NY Acad Sci., 1082:9-17 (2006); and Kawata et al. Mol Cancer Ther., 7(9):2904-12 (2008); each of which is incorporated herein in their entirety. Exemplary interfering nucleic acid delivery systems are provided in U.S. Pat. Nos. 8,283,461, 8,313,772, 8,501,930. 8,426,554, 8,268,798 and 8,324,366, each of which is hereby incorporated by reference in its entirety.

In some embodiments of the methods described herein, liposomes are used to deliver an inhibitory oligonucleotide to a subject. Liposomes suitable for use in the methods described herein can be formed from standard vesicle-forming lipids, which generally include neutral or negatively charged phospholipids and a sterol, such as cholesterol. The selection of lipids is generally guided by consideration of factors such as the desired liposome size and half-life of the liposomes in the blood stream. A variety of methods are known for preparing liposomes, for example, as described in Szoka et al. (1980), Ann. Rev. Biophys. Bioeng. 9:467; and U.S. Pat. Nos. 4,235,871, 4,501,728, 4,837,028, and 5,019,369, the entire disclosures of which are herein incorporated by reference.

The liposomes for use in the present methods can also be modified so as to avoid clearance by the mononuclear macrophage system (“MMS”) and reticuloendothelial system (“RES”). Such modified liposomes have opsonization-inhibition moieties on the surface or incorporated into the liposome structure.

Opsonization-inhibiting moieties for use in preparing the liposomes described herein are typically large hydrophilic polymers that are bound to the liposome membrane. As used herein, an opsonization inhibiting moiety is “bound” to a liposome membrane when it is chemically or physically attached to the membrane, e.g., by the intercalation of a lipid-soluble anchor into the membrane itself, or by binding directly to active groups of membrane lipids. These opsonization-inhibiting hydrophilic polymers form a protective surface layer that significantly decreases the uptake of the liposomes by the MMS and RES; e.g., as described in U.S. Pat. No. 4,920,016, the entire disclosure of which is herein incorporated by reference.

In some embodiments, opsonization inhibiting moieties suitable for modifying liposomes are water-soluble polymers with a number-average molecular weight from about 500 to about 40,000 daltons, or from about 2,000 to about 20,000 daltons. Such polymers include polyethylene glycol (PEG) or polypropylene glycol (PPG) derivatives; e.g., methoxy PEG or PPG, and PEG or PPG stearate; synthetic polymers such as polyacrylamide or poly N-vinyl pyrrolidone; linear, branched, or dendrimeric polyamidoamines; polyacrylic acids; polyalcohols, e.g., polyvinylalcohol and polyxylitol to which carboxylic or amino groups are chemically linked, as well as gangliosides, such as ganglioside GM1. Copolymers of PEG, methoxy PEG, or methoxy PPG, or derivatives thereof, are also suitable. In addition, the opsonization inhibiting polymer can be a block copolymer of PEG and either a polyamino acid, polysaccharide, polyamidoamine, polyethyleneamine, or polynucleotide. The opsonization inhibiting polymers can also be natural polysaccharides containing amino acids or carboxylic acids, e.g., galacturonic acid, glucuronic acid, mannuronic acid, hyaluronic acid, pectic acid, neuraminic acid, alginic acid, carrageenan; aminated polysaccharides or oligosaccharides (linear or branched); or carboxylated polysaccharides or oligosaccharides, e.g., reacted with derivatives of carbonic acids with resultant linking of carboxylic groups. In some embodiments, the opsonization-inhibiting moiety is a PEG, PPG, or derivatives thereof. Liposomes modified with PEG or PEG-derivatives are sometimes called “PEGylated liposomes.”

### CRISPR/Gene Editing

In some embodiments, the agent disclosed herein is an agent for genome editing (e.g., an agent used to delete at least a portion of a gene that encodes an L-dopa decarboxylase). Deletion of DNA may be performed using gene therapy to knock-out or disrupt the target gene. As used herein, a “knock-out” can be a gene knock-down or the gene can be knocked out by a mutation such as, a point mutation, an insertion, a deletion, a frameshift, or a missense mutation by techniques known in the art, including, but not limited to, retroviral gene transfer. In some embodiments, the agent is a nuclease (e.g., a zinc finger nuclease or a TALEN). Zinc-finger nucleases (ZFNs) are artificial restriction enzymes generated by fusing a zinc finger DNA-binding domain to a DNA-cleavage domain. Zinc finger domains can be engineered to target desired DNA sequences, which enable zinc-finger nucleases to target unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs). A TALEN is composed of a TALE DNA binding domain for sequence-specific recognition fused to the catalytic domain of an endonuclease that introduces double-strand breaks (DSB). The DNA binding domain of a TALEN is capable of targeting with high precision a large recognition site (for instance, 17 bp). Meganucleases are sequence-specific endonucleases, naturally occurring “DNA scissors,” originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes).

In another embodiment, the agent comprises a CRISPR-Cas9 guided nuclease and/or a sgRNA (Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339(6121): 819-23 (2013); and Gaj et al., “ZFN, TALEN, and CRISPR/Cas-based Methods for Genome Engineering,” Cell 31(7):397-405 (2013), which are hereby incorporated by reference in their entirety). Like the TALENs and ZFNs, CRISPR-Cas9 interference is a genetic technique which allows for sequence-specific control of gene expression in prokaryotic and eukaryotic cells by guided nuclease double-stranded DNA cleavage. It is based on the bacterial immune system—derived CRISPR (clustered regularly interspaced palindromic repeats) pathway. In some embodiments, the agent is an sgRNA. An sgRNA combines tracrRNA and crRNA, which are separate molecules in the native CRISPR/Cas9 system, into a single RNA construct, simplifying the components needed to use CRISPR/Cas9 for genome editing. In some embodiments, the crRNA of the sgRNA has complementarity to at least a portion of a gene that encodes an L-dopa decarboxylase. In some embodiments, the sgRNA may target at least a portion of a gene that encodes an L-dopa decarboxylase.

### Antibody Agents

In certain embodiments, the methods and compositions provided herein relate to antibodies and antigen binding fragments thereof that bind specifically to an L-dopa decarboxylase described herein. In some embodiments, the antibodies inhibit the activity of said L-dopa decarboxylase. Such antibodies can be polyclonal or monoclonal and can be, for example, murine, chimeric, humanized or fully human.

Polyclonal antibodies can be prepared by immunizing a suitable subject (e.g. a mouse) with a polypeptide antigen (e.g., a polypeptide having a sequence of an L-dopa decarboxylase or a fragment thereof). The polypeptide antibody titer in the immunized subject can be monitored over time by standard techniques, such as with an enzyme linked immunosorbent assay (ELISA) using immobilized polypeptide. If desired, the antibody directed against the antigen can be isolated from the mammal (e.g., from the blood) and further purified by well-known techniques, such as protein A chromatography to obtain the IgG fraction.

At an appropriate time after immunization, e.g., when the antibody titers are highest, antibody-producing cells can be obtained from the subject and used to prepare monoclonal antibodies using standard techniques, such as the hybridoma technique originally described by Kohler and Milstein (1975) Nature 256:495-497) (see also Brown et al. (1981) J. Immunol. 127:539-46; Brown et al. (1980) J. Biol. Chem. 255:4980-83; Yeh et al. (1976) Proc. Natl. Acad. Sci. 76:2927-31; and Yeh et al. (1982) Int. J. Cancer 29:269-75), the more recent human B cell hybridoma technique (Kozbor et al. (1983) Immunol. Today 4:72), the EBV-hybridoma technique (Cole et al. (1985) Monoclonal Antibodies and Cancer Therapy, Alan R. Liss, Inc., pp. 77-96) or trioma techniques. The technology for producing monoclonal antibody hybridomas is well known (see generally Kenneth, R. H. in Monoclonal Antibodies: A New Dimension In Biological Analyses, Plenum Publishing Corp., New York, New York (1980); Lerner, E. A. (1981) Yale J. Biol. Med. 54:387-402; Gefter, M. L. et al. (1977) Somatic Cell Genet. 3:231-36). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds to the polypeptide antigen, preferably specifically.

As an alternative to preparing monoclonal antibody-secreting hybridomas, a monoclonal specific for a receptor or ligand provided herein can be identified and isolated by screening a recombinant combinatorial immunoglobulin library (e.g., an antibody phage display library or an antibody yeast display library) with the appropriate polypeptide (e.g. a polypeptide having a sequence of an L-dopa decarboxylase or a fragment thereof) to thereby isolate immunoglobulin library members that bind the polypeptide.

Additionally, recombinant antibodies specific for a receptor or ligand provided herein, such as chimeric or humanized monoclonal antibodies, can be made using standard recombinant DNA techniques. Such chimeric and humanized monoclonal antibodies can be produced by recombinant DNA techniques known in the art, for example using methods described in U.S. Pat. Nos. 4,816,567; 5,565,332; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. 84:214-218; Nishimura et al. (1987) Cancer Res. 47:999-1005; Wood et al. (1985) Nature 314:446-449; and Shaw et al. (1988) J. Natl. Cancer Inst. 80:1553-1559); Morrison, S. L. (1985) Science 229:1202-1207; Oi et al. (1986) Biotechniques 4:214; Winter U.S. Pat. No. 5,225,539; Jones et al. (1986) Nature 321:552-525; Verhoeyan et al. (1988) Science 239:1534; and Beidler et al. (1988) J. Immunol. 141:4053-4060.

Human monoclonal antibodies specific for a receptor or ligand provided herein can be generated using transgenic or transchromosomal mice carrying parts of the human immune system rather than the mouse system. For example, “HuMAb mice” which contain a human immunoglobulin gene miniloci that encodes unrearranged human heavy (μ and γ) and κ light chain immunoglobulin sequences, together with targeted mutations that inactivate the endogenous μ and κ chain loci (Lonberg, N. et al. (1994) Nature 368(6474): 856 859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N. et al. (1994), supra; reviewed in Lonberg, N. (1994) Handbook of Experimental Pharmacology 113:49 101; Lonberg, N. and Huszar, D. (1995) Intern. Rev. Immunol. Vol. 13: 65 93, and Harding, F. and Lonberg, N. (1995) Ann. N. Y Acad. Sci 764:536 546). The preparation of HuMAb mice is described in Taylor, L. et al. (1992) Nucleic Acids Research 20:6287 6295; Chen, J. et al. (1993) International Immunology 5: 647 656; Tuaillon et al. (1993) Proc. Natl. Acad. Sci USA 90:3720 3724; Choi et al. (1993) Nature Genetics 4:117 123; Chen, J. et al. (1993) EMBO J. 12: 821 830; Tuaillon et al. (1994) J. Immunol. 152:2912 2920; Lonberg et al., (1994) Nature 368(6474): 856 859; Lonberg, N. (1994) Handbook of Experimental Pharmacology 113:49 101; Taylor, L. et al. (1994) International Immunology 6: 579 591; Lonberg, N. and Huszar, D. (1995) Intern. Rev. Immunol. Vol. 13: 65 93; Harding, F. and Lonberg, N. (1995) Ann. N.Y. Acad. Sci 764:536 546; Fishwild, D. et al. (1996) Nature Biotechnology 14: 845 851. See further, U.S. Pat. Nos. 5,545,806; 5,569,825; 5,625,126; 5,633,425; 5,789,650; 5,877,397; 5,661,016; 5,814,318; 5,874,299; 5,770,429; and 5,545,807.

In certain embodiments, the antibodies provided herein are able to bind to an L-dopa decarboxylase described herein with a dissociation constant of no greater than 10⁻⁶, 10⁻⁷, 10⁻⁸ or 10⁻⁹M. Standard assays to evaluate the binding ability of the antibodies are known in the art, including for example, ELISAs, Western blots and RIAs. The binding kinetics (e.g., binding affinity) of the antibodies also can be assessed by standard assays known in the art, such as by Biacore analysis. In some embodiments, the binding of the antibody to a receptor described herein substantially inhibits the activity of an L-dopa decarboxylase. As used herein, an antibody substantially inhibits the activity of the L-dopa decarboxylase when an excess of polypeptide reduces the activity of the L-dopa decarboxylase by at least about 20%, 40%, 60% or 80%, 85% or 90% (as measured in an in vitro competitive binding assay).

### Proteins

In certain embodiments, the compositions and methods provided herein relate to polypeptides that specifically bind to at least one L-dopa decarboxylase and are capable of inhibiting the activity of or decreasing the levels of at least one L-dopa decarboxylase (e.g., an L-dopa decarboxylase disclosed herein). In some embodiments, the peptide specifically binds to a TyrDC protein or fragment thereof.

In some embodiments, the polypeptides and proteins described herein can be isolated from cells or tissue sources by an appropriate purification scheme using standard protein purification techniques. In another embodiment, polypeptides and proteins described herein are produced by recombinant DNA techniques. Alternatively, polypeptides described herein can be chemically synthesized using standard peptide synthesis techniques.

In some embodiments, provided herein are chimeric or fusion proteins. As used herein, a “chimeric protein” or “fusion protein” comprises a polypeptide or protein described herein linked to a distinct polypeptide to which it is not linked in nature. For example, the distinct polypeptide can be fused to the N-terminus or C-terminus of the polypeptide either directly, through a peptide bond, or indirectly through a chemical linker. In some embodiments, the peptide described herein is linked to an immunoglobulin constant domain (e.g., an IgG constant domain, such as a human IgG constant domain).

A chimeric or fusion polypeptide described herein can be produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, Ausubel et al., eds., John Wiley & Sons: 1992). Moreover, many expression vectors are commercially available that already encode a fusion moiety.

The polypeptides and proteins described herein can be produced in prokaryotic or eukaryotic host cells by expression of polynucleotides encoding a polypeptide(s) described herein. Alternatively, such peptides can be synthesized by chemical methods. Methods for expression of heterologous polypeptides in recombinant hosts, chemical synthesis of polypeptides, and in vitro translation are well known in the art and are described further in Maniatis et al., Molecular Cloning: A Laboratory Manual (1989), 2nd Ed., Cold Spring Harbor, N.Y.; Berger and Kimmel, Methods in Enzymology, Volume 152, Guide to Molecular Cloning Techniques (1987), Academic Press, Inc., San Diego, Calif.; Merrifield, J. (1969) J. Am. Chem. Soc. 91:501; Chaiken I. M. (1981) CRC Crit. Rev. Biochem. 11:255; Kaiser et al. (1989) Science 243:187; Merrifield, B. (1986) Science 232:342; Kent, S. B. H. (1988) Annu. Rev. Biochem. 57:957; and Offord, R. E. (1980) Semisynthetic Proteins, Wiley Publishing, which are incorporated herein by reference.

### Pharmaceutical Compositions

In certain embodiments, provided herein is a composition, e.g., a pharmaceutical composition, containing at least one agent described herein together with a pharmaceutically acceptable carrier. In one embodiment, the composition includes a combination of multiple (e.g., two or more) agents described herein. In some embodiments, the composition further comprises administering a second agent that inhibits an L-dopa decarboxylase. The second agent may be carbidopa (Lodosyn, Sinemet, Pharmacopa, Atamet, Stalevo, etc.), benserazide (Madopar, Prolopa, Modopar, Madopark, Neodopasol, EC-Doparyl, etc.), methyldopa (Aldomet, Aldoril, Dopamet, Dopegyt, etc.), DFMD, or 3′,4′,5,7-Tetrahydroxy-8-methoxyisoflavone [58262-89-8]. The second agent may be any agent that inhibits the activity of or decreases the levels of an enzyme that dehydroxylates dopamine. The second agent may an agent that inhibits the activity of or decreases the levels of an enzyme that dehydroxylates dopamine (e.g., bis-molybdopterin guanine dinucleotide cofactor (moco)-containing enzyme). The method may comprise administering a second agent to the subject that increases the activity of or increases the levels of an enzyme that dehydroxylates dopamine.

As described in detail below, the pharmaceutical compositions disclosed herein may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; or (2) parenteral administration, for example, by subcutaneous, intramuscular, intravenous, intrathecal, intracerebral or epidural injection as, for example, a sterile solution or suspension, or sustained-release formulation.

Methods of preparing these formulations or compositions include the step of bringing into association an agent described herein with the carrier and, optionally, one or more accessory ingredients. In general, the formulations are prepared by uniformly and intimately bringing into association an agent described herein with liquid carriers, or finely divided solid carriers, or both, and then, if necessary, shaping the product.

Pharmaceutical compositions suitable for parenteral administration comprise one or more agents described herein in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions include water, ethanol, dimethyl sulfoxide (DMSO), polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Regardless of the route of administration selected, the agents provided herein, which may be used in a suitable hydrated form, and/or the pharmaceutical compositions disclosed herein, are formulated into pharmaceutically-acceptable dosage forms by conventional methods known to those of skill in the art. In some embodiments, the agent decreases the level of or inhibits the activity of an L-dopa decarboxylase by at least 10%, least 15%, at least 25%, at least 30%, at least 35%, at least 40%, at least 45%, at least 50%, at least 55%, at least 60, %,at least 65%, at least 70%, at least 75%, at least 80%, at least 85%, at least 90%, at least 95%, or at least 99%.

### Methods

Provided herein are methods and compositions related to preventing or treating a condition (e.g., a condition or indication disclosed herein) in a subject comprising administering an agent that inhibits the activity of or decreases the levels of L-dopa decarboxylase conjointly with levodopa (L-dopa). Also provided herein are methods of treating Parkinson's Disease in a subject and/or method of treating or preventing symptoms resulting from dopaminergic neuron loss in a subject in need thereof by administering an agent to the subject that inhibits the activity of or decreases the levels of TyrDC conjointly with levodopa.

The L-dopa decarboxylase may be tyrosine decarboxylase (TyrDC). The L-dopa decarboxylase may be any a PLP-dependent tyrosine decarboxylase. In some embodiments, the agent preferentially inhibits the activity of or decreases the level of TyrDC over amino acid decarboxylase (AADC).

In some embodiments, the condition is Parkinsonism, Parkinson's disease, corticobasal degeneration (CBD), dementia with Lewy bodies (DLB), essential tremor, multiple system atrophy (MSA), progressive supranuclear palsy (PSP), vascular (arteriosclerotic) parkinsonism, Parkinson's-like symptoms that develop after encephalitis, injury to the nervous system caused by carbon monoxide poisoning, or injury to the nervous system caused by manganese poisoning.

The method included herein may also comprise further administering carbidopa and/or benserazide to the subject. The method may comprise administering an agent that to the subject that inhibits the activity of or decreases the levels of an enzyme that dehydroxylates dopamine (e.g., bis-molybdopterin guanine dinucleotide cofactor (moco)-containing enzyme).

In some embodiments, the agent preferentially inhibits the activity of or decreases the level of TyrDC over AADC.

In some embodiments, the agent decreases the level of or inhibits the activity of a TyrDC protein by at least 5%, by at least 10%, by at least 15%, by at least 20%, by at least 25%, by at least 30%, by at least 35%, by at least 40%, at least 45%, by at least 50%, by at least 55%, by at least 60%, by at least 65%, by at least 70%, by at least 75%, or by at least 80%, by at least 85%, by at least 90%, by at least 95%, or by at least 99%.

The agent may be administered to the subject systemically, intravenously, subcutaneously, intramuscularly.

Also provided herein are in-vitro methods of determining whether an agent is a therapeutic agent for Parkinson's Disease comprising determining whether the test agent decreases the levels of or inhibits the activity of a TyrDC enzyme, wherein the test agent is determined to be a therapeutic agent for the treatment of Parkinson's Disease if the test agent decreases the levels of or inhibits the activity of a TyrDC enzyme. The test agent may be a member of a library of test agents. The test agent may be an interfering nucleic acid, a peptide, a small molecule, or an antibody. The agent may decrease the level of or inhibits the activity of the TyrDC enzyme by at least 10%, at least 20%, at least 30%, at least 40%, at least 50%, at least 75%, or at least 95%.

Provided herein are methods of treating a condition in a subject, comprising administering a composition that inhibits the activity of or decreases the levels of a bacteria that expresses a PLP-dependent tyrosine decarboxylase (TyrDC) or a TyrDC homolog conjointly with levodopa. The bacteria that expresses a PLP-dependent tyrosine decarboxylase may be Enterococcus (e.g., Enterococcus faecalis). The bacteria may express a PLP-dependent tyrosine decarboxylase may be Enterococcus faecium. The bacteria may be any bacteria that expresses a PLP-dependent tyrosine decarboxylase (e.g., TyrDC or a TryDC homolog). The bacteria that expresses a PLP-dependent tyrosine decarboxylase may be Proteobacteria. The bacteria that expresses a PLP-dependent tyrosine decarboxylase may be Lactobacillus. The bacteria that expresses a PLP-dependent tyrosine decarboxylase may be Providencia (e.g., Providencia rettgeri). The bacteria that expresses a PLP-dependent tyrosine decarboxylase may be Proteus (e.g., Proteus mirabilis).

As used herein, TyrDC may refer to TyrDC or a TryDC homolog.

The method may further comprise administering a composition that inhibits the activity of or decreases the levels of bacteria that expresses a molybdenum-dependent enzyme. In some embodiments, the bacteria that expresses a molybdenum-dependent enzyme is Eggerthella lenta.

Typical subjects for treatment include persons afflicted with or suspected of having or being pre-disposed to a disease disclosed herein, or persons susceptible to, suffering from or that have suffered a disease disclosed herein. A subject may or may not have a genetic predisposition for a disease disclosed herein. In some embodiments, disclosed herein are methods which comprise administration of an agent disclosed herein (e.g., a small molecule or inhibitory nucleic acid) conjointly with a compound for treating a condition disclosed herein. As used herein, the phrase “conjoint administration” refers to any form of administration of two or more different agents (e.g., a composition disclosed herein and a nutrient disclosed herein) such that the second agent is administered while the previously administered agent is still effective in the body. For example, the compositions disclosed herein and the nutrients disclosed herein can be administered either in the same formulation or in a separate formulation, either concomitantly or sequentially.

The pharmaceutical compositions disclosed herein may be delivered by any suitable route of administration, including orally, locally, and parenterally. In certain embodiments the pharmaceutical compositions are delivered generally (e.g., via oral or parenteral administration).

In certain aspects, agents and/or compositions disclosed herein may be administered at a dose sufficient to achieve the desired result.

In certain embodiments, the method may comprise administering about 1 μg to about 1 gram of agent or composition to the subject, such as about 1 g to about 1 mg, about 2 μg to about 2 mg, about 3μg to about 3 mg, about 4 μg to about 4 mg, about 100 μg to about 2 mg, about 200 μg to about 2 mg, about 300 μg to about 3 mg, about 400 μg to about 4 mg, about 250 μg to about 1 mg, or about 250 μg to about 750 μg of the agent or composition. In some embodiments, the method may comprise administering about 25 about 50 about 75 μg/kg, about 100 μg/kg, about 125 μg/kg, about 150 μg/kg, about 175 μg/kg, about 200 μg/kg, about 225 μg/kg, about 250 μg/kg, about 275 μg/kg, about 300 μg/kg, about 325 μg/kg, about 350 μg/kg, about 375 μg/kg, about 400 μg/kg, about 425 μg/kg, about 450 μg/kg, about 475 μg/kg, about 500 μg/kg, about 600 μg/kg, about 650 μg/kg, about 700 μg/kg, about 750 μg/kg, about 800 μg/kg, about 850 μg/kg, about 900 μg/kg, about 950 μg/kg, about 1000 μg/kg, about 1200 μg/kg, about 1250 μg/kg, about 1300 μg/kg, about 1333 μg/kg, about 1350 μg/kg, about 1400 μg/kg, about 1500 μg/kg, about 1600 μg/kg, about 1750 μg/kg, about 1800 μg/kg, about 2000 μg/kg, about 2200 μg/kg, about 2250 μg/kg, about 2300 μg/kg, about 2333 μg/kg, about 2350 μg/kg, about 2400 μg/kg, about 2500 μg/kg, about 2667 μg/kg, about 2750 μg/kg, about 2800 μg/kg, about 3 mg/kg, about 3.5 mg/kg, about 3.5 mg/kg, about 4 mg/kg, about 4.5 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, or about 100 mg/kg. In some embodiments, the method may comprise administering about 1 mg/kg to about 10 mg/kg, about 10 mg/kg to about 20 mg/kg, about 20 mg/kg to about 50 mg/kg, about 50 mg/kg to about 100 mg/kg of the agent or composition. The dose may be titrated up down following initial administration to any effective dose.

In some embodiments, administering an agent or composition to the subject comprises administering a bolus of the composition. The method may comprise administering the composition to the subject at least once per month, twice per month, three times per month. In certain embodiments, the method may comprise administering the composition at least once per week, at least once every two weeks, or once every three weeks. In some embodiments, the method may comprise administering the composition to the subject 1, 2, 3, 4, 5, 6, or 7 times per week.

In some embodiments, the agents and/or compositions described herein may be administered conjointly with a second agent (e.g., a second agent disclosed herein).

In certain embodiments, the compositions of the invention can be administered in a variety of conventional ways. In some aspects, the compositions of the invention are suitable for parenteral administration. In some embodiments, these compositions may be administered, for example, intraperitoneally, intravenously, intrarenally, or intrathecally. In some aspects, the compositions of the invention are injected intravenously.

In some embodiments, actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

In general, a suitable daily dose of an agent described herein will be that amount of the agent which is the lowest dose effective to produce a therapeutic effect. Such an effective dose will generally depend upon the factors described above.

Agents useful in the methods disclosed herein may be identified, for example, using assays for screening candidate or test compounds which inhibit an L-dopa decarboxylase by testing for compounds that inhibit the activity of or decrease the levels of an L-dopa decarboxylase described herein. The basic principle of the assay systems used to identify compounds that inhibit an L-dopa decarboxylase activity include administering a test compound (e.g., a small molecule or an inhibitory nucleic acid) to a system or assay where the activity of an L-dopa decarboxylase and/or the level of a an L-dopa decarboxylase may be calculated prior and post administration of an L-dopa decarboxylase inhibitor to the system or assay. In order to test an agent for L-dopa decarboxylase modulatory activity, the reaction mixture is prepared in the presence and absence of the test compound. Control reaction mixtures are incubated without the test compound or with a placebo. The inhibition of an L-dopa decarboxylase or L-dopa decarboxylase levels is then detected. In some embodiments, the test agent is determined to be a therapeutic agent if the test agent decreases the levels of or inhibits the activity of an L-dopa decarboxylase. The test agent may be a member of a library of test agents. The agent may be an interfering nucleic acid, a peptide, a small molecule, an antibody, or any agent disclosed herein. The agent may decreases the level of or inhibits the activity of an L-dopa decarboxylase by at least 1%, at least 5%, at least 10%, at least 20%, at least 25%, at least 30%, at least 35%, at least 40%, at least 45%, at least 50%, at least 55%, at least 60%, at least 65%, at least 70% at least 75%, at least 80%, at least 85%, at least 90%, at least 95%, or at least 99%.

### Exemplification

A growing body of evidence links the trillions of microbes that inhabit the human gastrointestinal tract (the human gut microbiota) to neurological conditions, including the debilitating neurodegenerative disorder Parkinson's disease. Gut microbes from Parkinson's patients exacerbate motor deficits when transplanted into germ-free mouse models of disease. This effect is reversed with antibiotic treatment, suggesting a causal role for gut microbes in neurodegeneration. Multiple studies have revealed differences in gut microbiota composition in Parkinson's disease patients compared to healthy controls that may correlate with disease severity. However, the influence of the human gut microbiota on the treatment of Parkinson's and other neurodegenerative diseases remains poorly understood.

The primary treatment for Parkinson's disease is Levodopa (L-dopa), which is prescribed to manage motor symptoms resulting from dopaminergic neuron loss in the substantia nigra. After crossing the blood-brain barrier, L-dopa is decarboxylated by aromatic amino acid decarboxylase (AADC) to give dopamine, the active therapeutic agent. However, dopamine generated in the periphery by AADC cannot cross the blood-brain barrier, and only 1-5% of L-dopa reaches the brain due to extensive pre-systemic metabolism in the gut by enzymes including AADC. Peripheral production of dopamine also causes gastrointestinal side effects, can lead to orthostatic hypotension through activation of vascular dopamine receptors, and may induce cardiac arrhythmias. To decrease peripheral metabolism, L-dopa is co-administered with AADC inhibitors such as carbidopa. Despite this, 56% of L-dopa is metabolized peripherally, and patients display highly variable responses to the drug, including loss of efficacy over time.

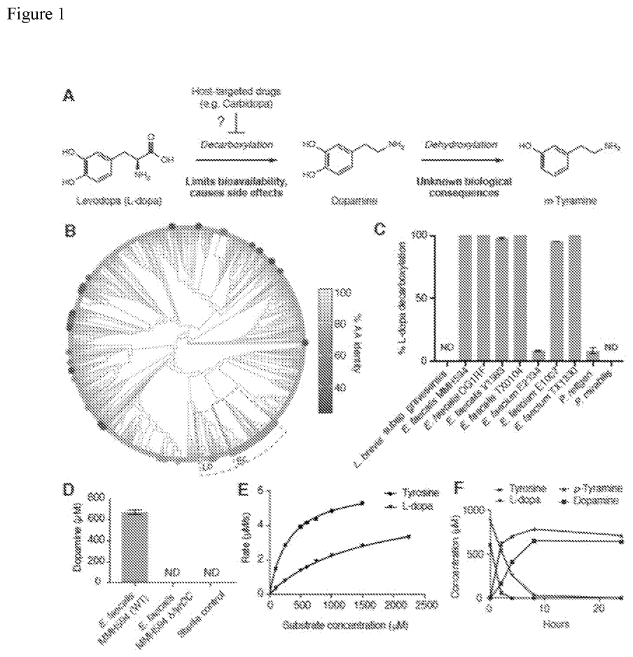

Administering broad-spectrum antibiotics improves L-dopa therapy, suggesting that gut bacteria interfere with drug efficacy. The gut microbiota can also metabolize L-dopa, potentially reducing its bioavailability and leading to side effects. The major proposed pathway involves an initial decarboxylation of L-dopa to dopamine followed by a uniquely microbial dehydroxylation reaction that converts this neurotransmitter to m-tyramine by selectively removing the para hydroxyl group of the catechol ring (FIG. 1, Panel A). When the work began, the gut microbial species, genes, and enzymes involved in these transformations were unknown as previous studies examined undefined and uncharacterized consortia. The clinical relevance of this pathway was also unclear given the potential effects of co-administered inhibitors of host peripheral L-dopa metabolism on these gut microbial activities.

### The Human Gut Bacterium Enterococcus faecalis Decarboxylates L-dopa

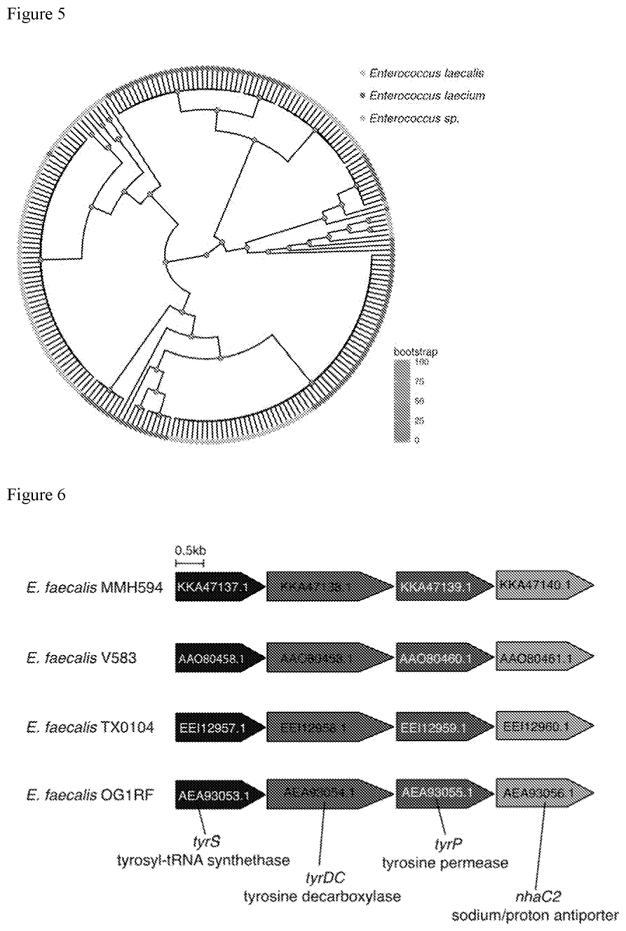

Using a genome mining approach, strains encoding candidate L-dopa decarboxylating enzymes were identified. Aromatic amino acid decarboxylation is typically performed by enzymes employing pyridoxal-5′-phosphate (PLP), an organic cofactor that provides an electron sink. A PLP-dependent tyrosine decarboxylase (TyrDC) from the food-associated strain Lactobacillus brevis CGMCC 1.2028 was shown to have promiscuous activity toward L-dopa in vitro. To locate TyrDC homologs in human gut bacteria, a BLASTP search was performed against the complete set of Human Microbiome Project (HMP) reference genomes available via NCBI. The majority of hits were found in the neighboring genus Enterococcus, with some hits within lactobacilli and Proteobacteria (FIG. 1, Panel B, FIG. 5, Data S1). Ten representative gut strains containing TyrDC homologs (29-100% amino acid ID) were selected and examined their ability to decarboxylate L-dopa in anaerobic culture. While both Enterococcus faecalis and Enterococcus faecium displayed activity, only E. faecalis showed complete decarboxylation across all strains tested (FIG. 1, Panel C). All E. faecalis strains tested share the highly conserved four-gene tyrDC operon (FIG. 6), and it was found tyrDC in 98.4% of the E. faecalis assemblies deposited in NCBI with a median amino acid identity of 99.8 (range 97.0-100). This high degree of sequence conservation and prevalence is consistent with tyrosine decarboxylation being a common phenotypic trait of E. faecalis. This prevalent, genetically tractable gut organism was therefore chosen as a model for characterizing L-dopa decarboxylation.

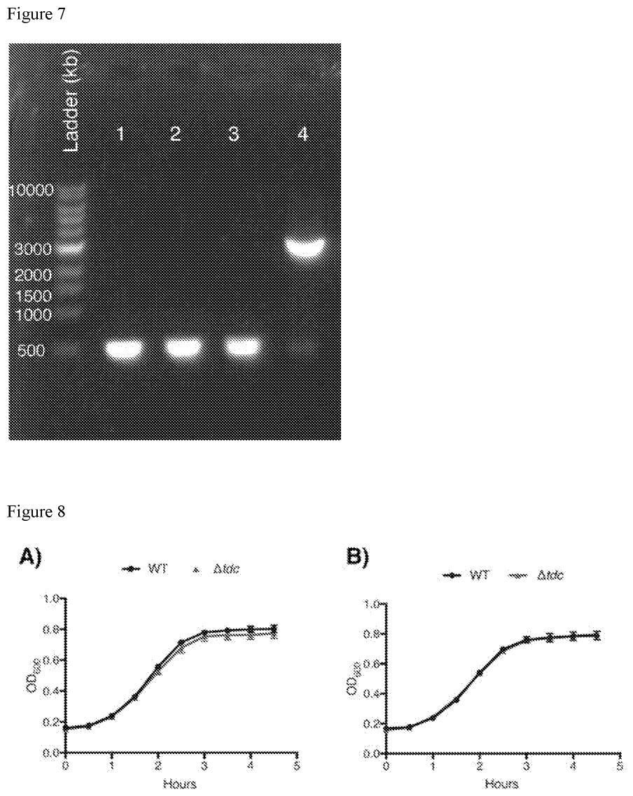

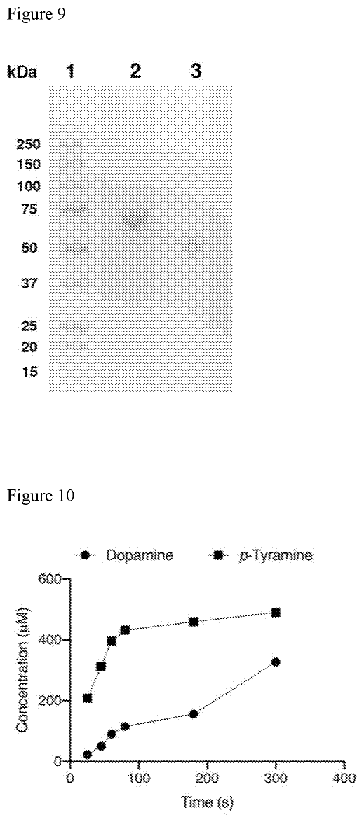

The connection between tyrDC and L-dopa decarboxylation was unknown. Genetics and in vitro biochemistry experiments were used to confirm that TyrDC is necessary and sufficient for L-dopa decarboxylation by E. faecalis. E. faecalis MMH594 mutants carrying a 2 kb Tet-cassette disrupting tyrDC could not decarboxylate L-dopa (FIG. 1, Part D and FIG. 7) and displayed no growth defects compared to wild-type (FIG. 8). In vitro characterization of TyrDC revealed a 5-fold higher catalytic efficiency towards L-tyrosine compared to L-dopa, suggesting drug metabolism arises from promiscuous enzyme activity (FIG. 1, Panel E, FIG. 9 and Table 1). This selectivity contrasts sharply with that of AADC, which displays very low activity towards L-tyrosine. Though TyrDC from E. faecalis was previously shown to decarboxylate tyrosine and phenylalanine, its ability to accept L-dopa had not been demonstrated.

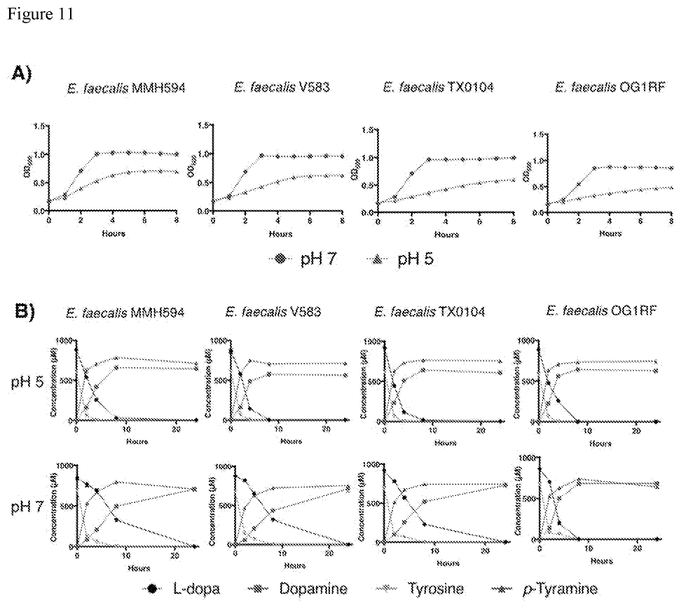

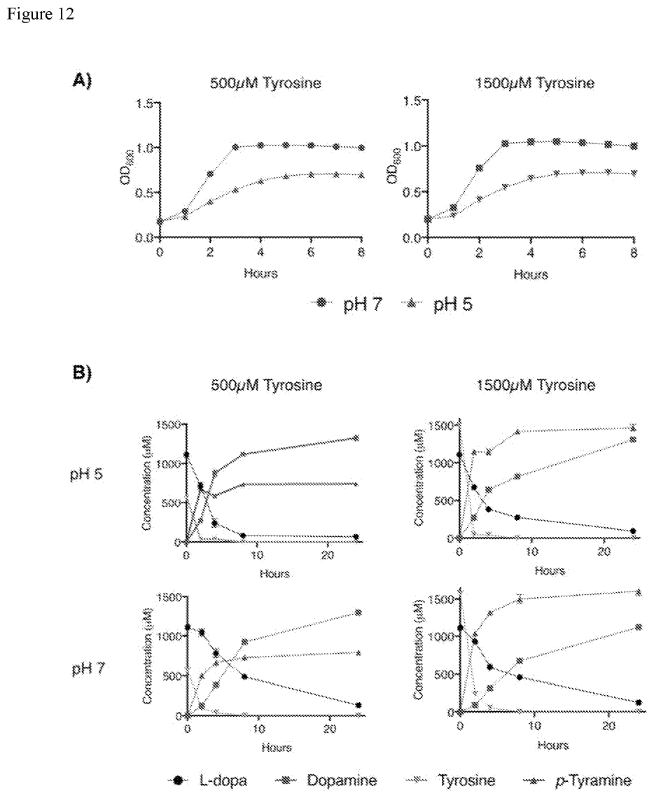

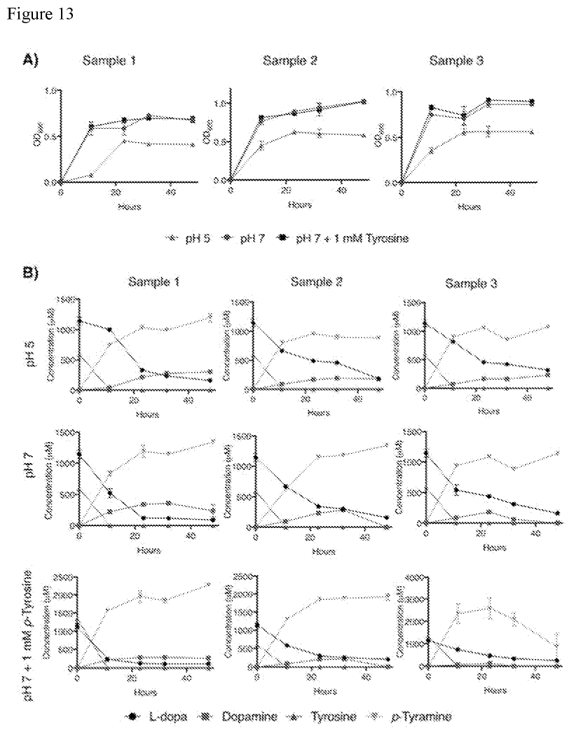

Tyrosine was tested next, which is the preferred substrate for TyrDC and is present in the small intestine, could interfere with L-dopa decarboxylation by E. faecalis. In competition experiments, purified TyrDC (FIG. 10) and anaerobic E. faecalis cultures decarboxylated L-dopa and tyrosine simultaneously (500 μM tyrosine, approximating the resting small intestinal concentration) (FIG. 1, Panel F and FIG. 11). This observation sharply contrasts with previous investigations of phenylalanine, which is metabolized by E. faecalis only when tyrosine is completely consumed. Simultaneous decarboxylation of L-dopa and tyrosine also occurred in E. faecalis MMH594 cultures containing higher tyrosine concentrations (1.5 mM, approximating the small intestinal post-meal concentration) (FIG. 12) and in three human fecal suspensions (FIG. 13). As observed previously for tyrosine, L-dopa decarboxylation occurred more rapidly at lower pH across all strains tested (FIGS. 11 and 12), suggesting this metabolism is likely accelerated at the lower pH of the upper small intestine. As the Kₘ of TyrDC for L-dopa (1.5 mM) is below the estimated maximum in vivo small intestinal L-dopa concentration even at its lowest clinically administered dose (5 mM), these data strongly suggest that peripheral decarboxylation is performed by both host and gut bacterial enzymes.

### Eggerthella lenta Dehydroxylates Dopamine Using a Molybdenum-Dependent Enzyme

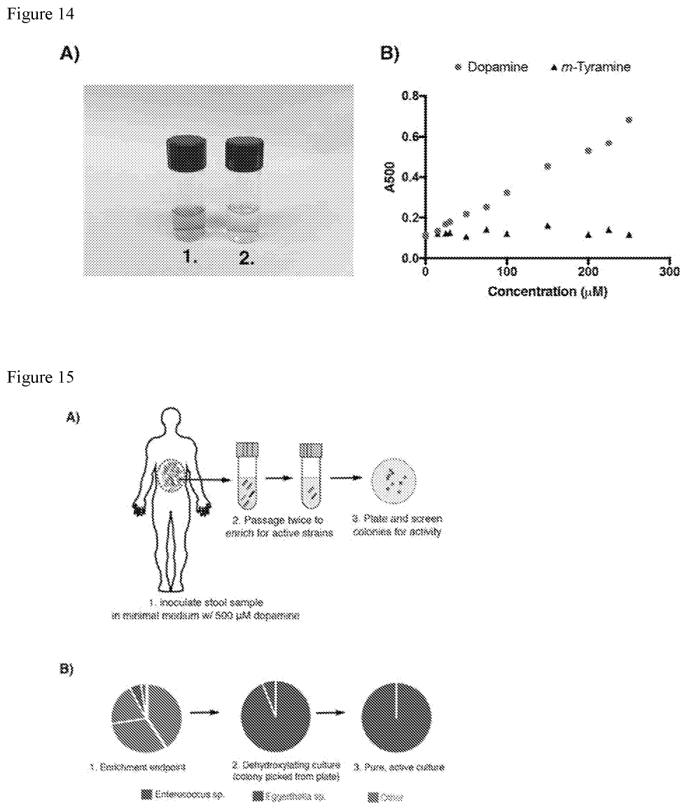

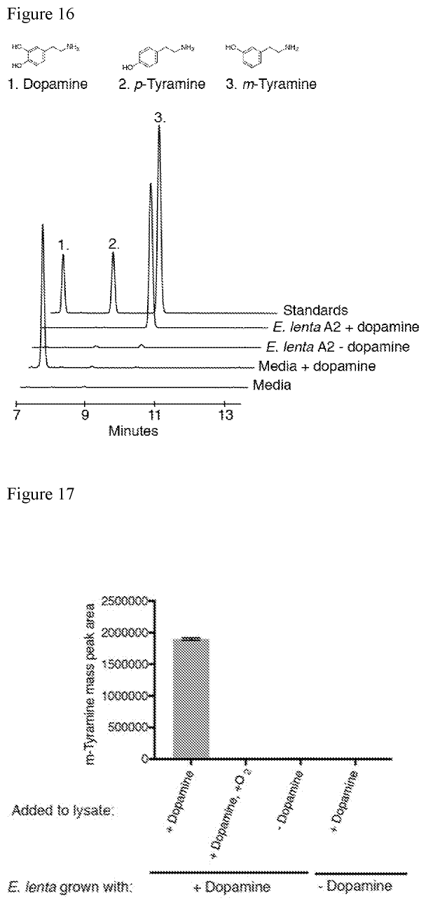

Having identified a gut bacterial L-dopa decarboxylase, the conversion of dopamine to m-tyramine was examined next as this activity may influence the side effects associated with peripheral L-dopa decarboxylation. E. faecalis did not further metabolize dopamine, indicating this step was performed by a different microorganism. Dehydroxylation of dopamine has not been reported for any bacterial isolate, and a screen of 18 human gut strains failed to uncover metabolizers. Therefore, enrichment culturing was used to obtain a dopamine dehydroxylating organism. Recognizing the chemical parallels between this reductive dehydroxylation and reductive dehalogenation of chlorinated aromatics, which enables anaerobic respiration in certain bacteria, a stool sample was inoculated from a human donor into a minimal growth medium containing 0.5 mM dopamine as the sole electron acceptor (FIGS. 14 and 15). Passaging over multiple generations enriched for active strains as assessed by a colorimetric assay for catechol dehydroxylation (FIG. 15). This effort identified a strain of the gut Actinobacterium Eggerthella lenta (referred to herein as strain A2) capable of selectively removing the para hydroxyl group of dopamine to give m-tyramine (FIG. 16). As E. lenta also inactivates the cardiac drug digoxin, these finding suggests a wider role for this gut organism in drug metabolism.

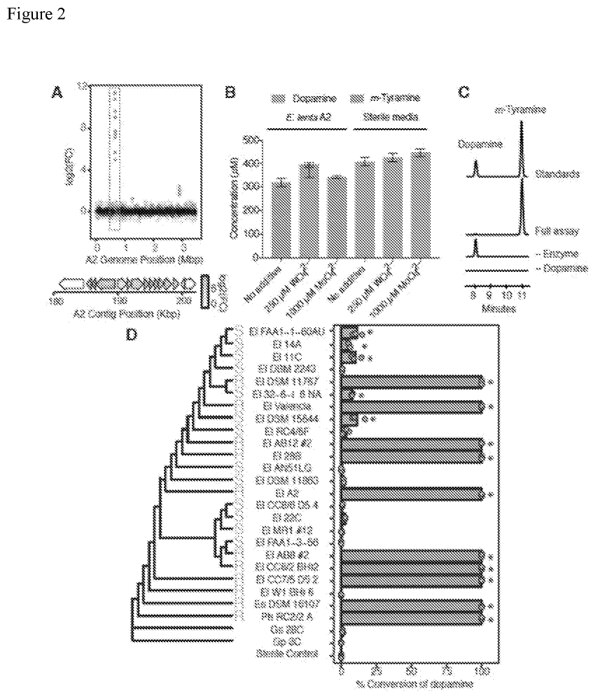

Catechol dehydroxylation is a chemically challenging reaction that has no equivalent in synthetic chemistry and likely involve unusual enzymology. To identify the dopamine dehydroxylating enzyme, the E. lenta A2 genome was first searched for genes encoding homologs of the only characterized aromatic para-dehydroxylase, 4-hydroxybenzoyl-CoA reductase, but found no hits. Assays with E. lenta A2 cell lysates showed dopamine dehydroxylation required anaerobic conditions and was induced by dopamine (FIG. 17). RNA-sequencing of E. lenta A2 was used to identify the dehydroxylase. This experiment revealed >2,500-fold upregulation of 3 co-localized genes in response to dopamine (FIG. 2, Panel A and Table 2). These genes encode a predicted bis-molybdopterin guanine dinucleotide cofactor (moco)-containing enzyme belonging to the DMSO reductase family. Moco-dependent enzymes catalyze a wide variety of oxygen-transfer reactions but have not been implicated in catechol dehydroxylation. It was therefore hypothesized that this enzyme was a dopamine dehydroxylase (Dadh).

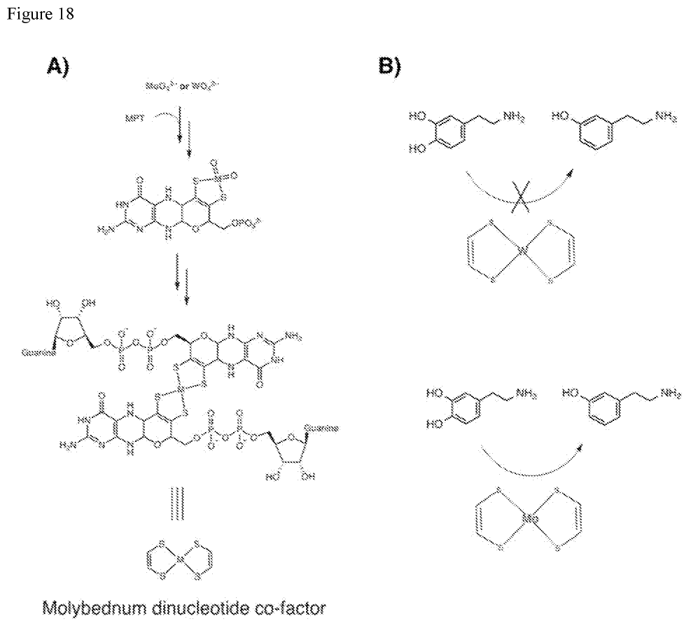

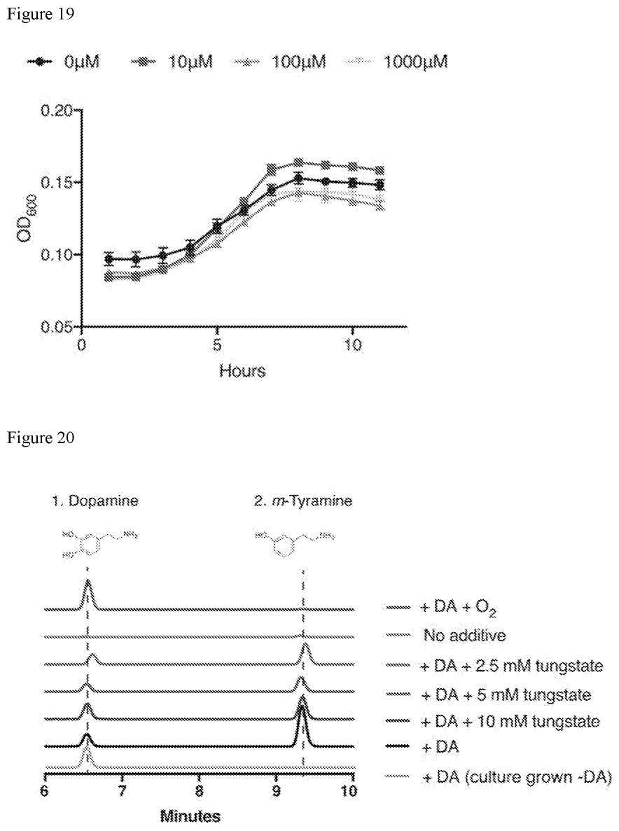

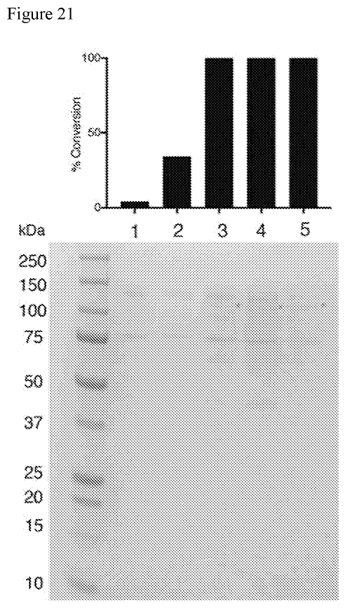

To assess Dadh's role in dopamine dehydroxylation, it was first explored whether this activity was molybdenum-dependent by culturing E. lenta A2 in the presence of tungstate. Substitution of molybdate with tungstate during moco biosynthesis generates an inactive metallocofactor (FIG. 18). Indeed, treating cultures of E. lenta A2 with tungstate inhibited dopamine dehydroxylation without affecting growth (FIG. 2, Panel B, and FIG. 19) while incubating cell lysates with tungstate had no effect, consistent with inhibition requiring active moco biosynthesis (FIG. 20). The activity of Dadh in vitro was then confirmed. Heterologous expression of >20 constructs in multiple hosts failed to provide active enzyme, prompting us to pursue native purification. Anaerobic activity-guided fractionation of E. lenta A2 cell lysates yielded a dopamine dehydroxylating fraction containing four proteins as assessed by SDS-PAGE (FIG. 2, Panel C, FIG. 21 and Table 3). Dehydroxylation activity correlated with a 115 kDa band that was confirmed to be Dadh using mass spectrometry. Importantly, Dadh was the only isolated protein upregulated in the presence of dopamine (Tables 2 and 3). Together, these data strongly support the assignment of this enzyme.

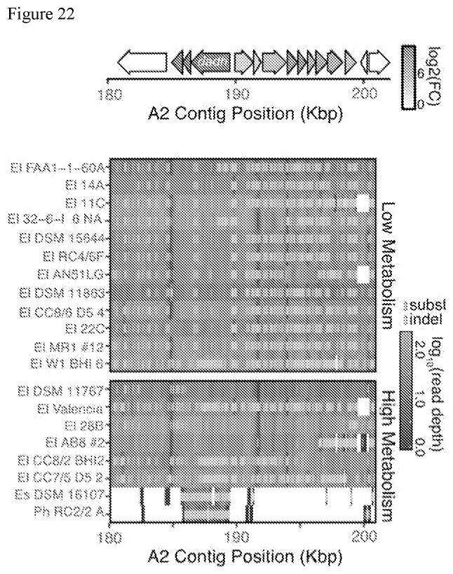

It was next assessed whether the presence of dadh in microbial genomes correlated with dopamine dehydroxylation. A BLASTP search revealed that this enzyme is restricted to E. lenta and its close Actinobacterial relatives (Table 4), prompting us to screen a collection of 26 gut Actinobacterial isolates for their ability to dehydroxylate dopamine in anaerobic culture. Though Dadh appeared to be encoded by 24 of the 26 strains (92-100% amino acid ID, FIG. 22 and table 5), only 10 Eggerthella strains quantitatively converted dopamine to m-tyramine, with low (<11%) or no metabolism in the others (FIG. 2, Panel D). This strain-level variability in dopamine metabolism reinforces that gut microbial species identity is often not predictive of metabolic functions.

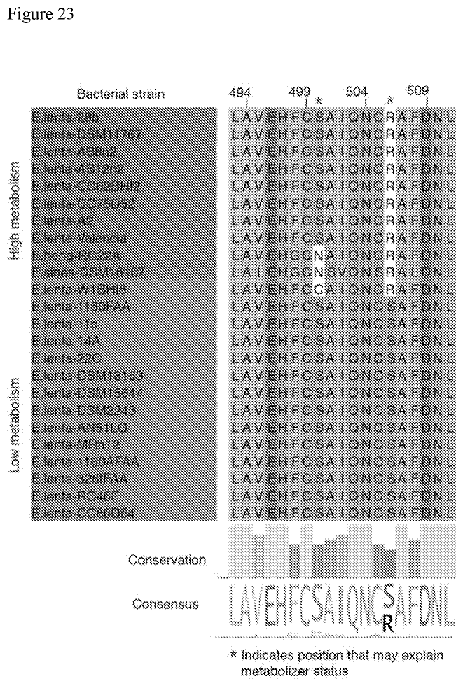

To better understand this variation, RNA-sequencing experiments were first performed with metabolizing (E. lenta 28b) and non-metabolizing (E. lenta DSM2243) strains in the presence and absence of dopamine. Surprisingly, dadh was upregulated in response to dopamine in both strains, indicating that lack of activity in E. lenta DSM2243 did not arise from differences in transcription (Tables 6 and 7). Aligning the Dadh protein sequences, it was instead found a single amino acid substitution that almost perfectly predicted metabolizer status: position 506 is an arginine in metabolizing strains and a serine in inactive strains (FIG. 2, Panel D and FIG. 23). This change arises from a single nucleotide polymorphism (SNP) in dadh. The only exception, E. lenta W1BHI6, has the Arg506 variant and an additional substitution nearby (Cys500) (FIG. 23). Thus, specific amino acid residues in the Dadh enzyme, rather than presence or transcription of dadh, predict dopamine dehydroxylation among gut bacterial strains. The Dadh variants do not correlate with E. lenta phylogeny (FIG. 2, Part D), suggesting that this activity has been gained/lost multiple times.

### E. faecalis and E. lenta Metabolize L-dopa in Human Gut Microbiotas

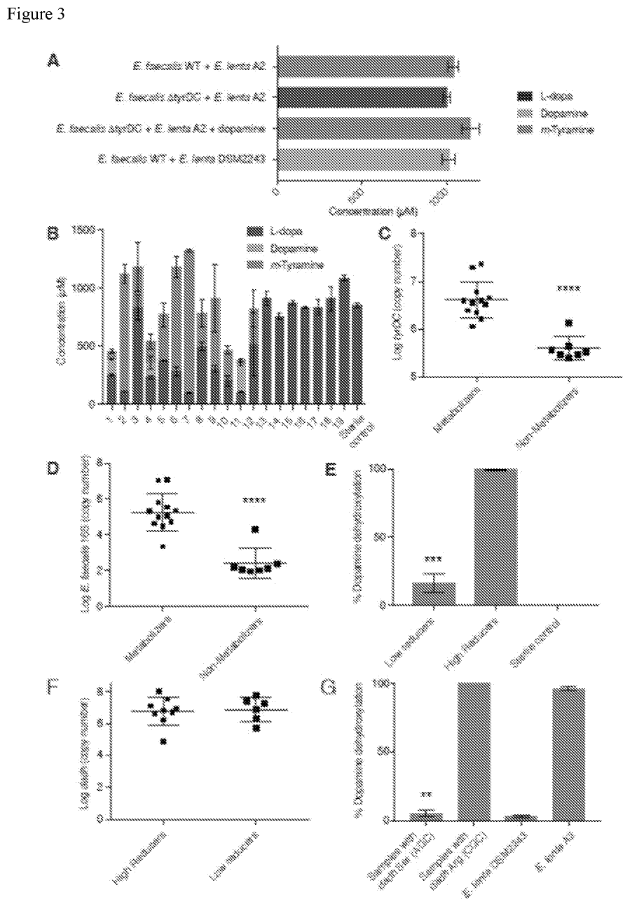

Having identified organisms and enzymes that perform the individual steps in the L-dopa pathway, it was then tested whether E. faecalis and E. lenta generated m-tyramine in co-culture. Wild-type E. faecalis grown with E. lenta A2 (Arg506) fully converted L-dopa to m-tyramine (FIG. 3, Panel A). While a co-culture containing the E. faecalis tyrDC mutant could not consume L-dopa, m-tyramine was produced when exogenous dopamine was added to this culture, revealing that E. lenta A2 was still metabolically active. Finally, incubating wild-type E. feacalis with the non-metabolizing E. lenta DSM2243 (Ser506) strain produced only dopamine, indicating that this Dadh variant is also inactive in a co-culture setting (FIG. 3, Panel A).

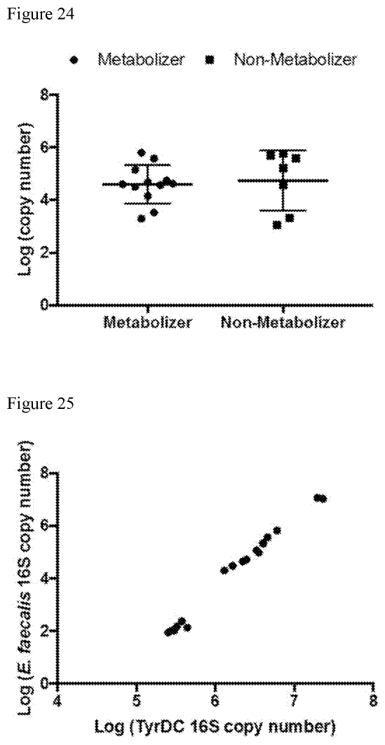

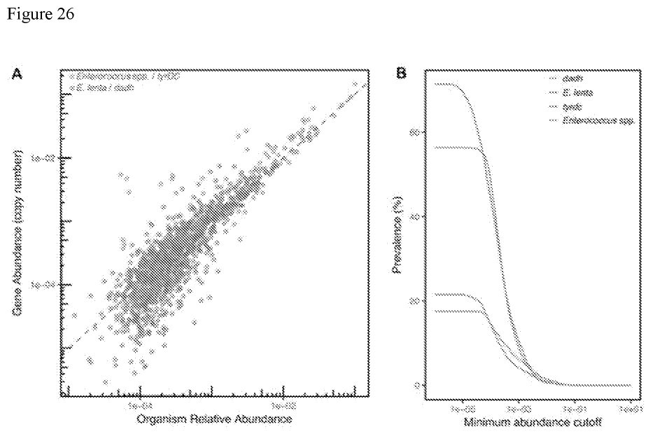

To investigate whether E. faecalis and E. lenta transform L-dopa in the human gut microbiota, the metabolism of deuterated L-dopa by fecal suspensions ex vivo was then assessed. While 7/19 samples did not show detectable depletion of L-dopa, the remaining samples displayed significant variability in metabolism, ranging from partial (25%) to almost full conversion (98%) of L-dopa to m-tyramine (FIG. 3, Panel B). Next, it was asked whether the abundance of tyrDC predicted metabolism in these samples. qPCR enumeration of tyrDC and E. faecalis discriminated metabolizing and non-metabolizing samples (p<0.0001, one-tailed Mann-Whitney test) (FIG. 3, Part C, D). In contrast, E. lenta abundance showed no association with L-dopa metabolism (FIG. 24). A strong linear correlation was then found between tyrDC abundance and E. faecalis abundance (R² =0.99, p<0.0001) (FIG. 25) which likely reflects the high conservation of tyrDC in E. faecalis genomes. These data also suggest that E. faecalis is the dominant microorganism responsible for L-dopa decarboxylation in these complex human gut microbial communities. Consistent with this, E. faecalis abundance significantly correlated with tyrDC abundance in 1870 human gut microbiomes (R^{2 >}0812, P<2.2e−16 Pearson's Correlation) (FIG. 26).

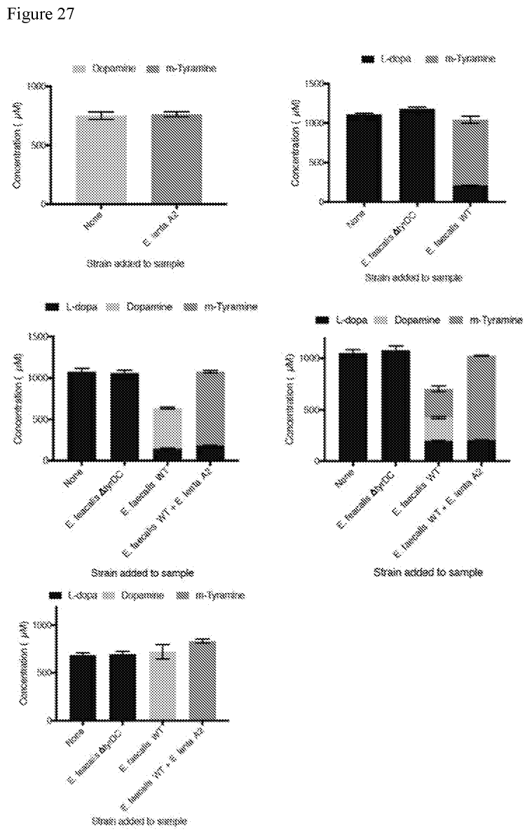

To confirm that E. faecalis could decarboxylate L-dopa in complex gut microbiotas, this organism was added to non-metabolizing samples. While introducing the tyrDC-deficient strain did not change L-dopa levels, including the wild-type strain led to complete depletion of L-dopa (FIG. 27, B to E). In some samples, addition of wild-type E. faecalis was sufficient to yield quantitative production of m-tyramine, indicating the presence of dopamine dehydroxylating organisms in these communities (FIG. 27, B and D). Finally, addition of both the wild-type E. faecalis and the metabolizing strain E. lenta A2 to non-metabolizing samples or the addition of E. lenta A2 alone to a decarboxylating sample generated m-tyramine (FIG. 27, A and C to E). Taken together, these data indicate that the abundance of E. faecalis and its encoded tyrDC predicts the considerable inter-individual variation in L-dopa metabolism observed in complex human gut microbiota samples.

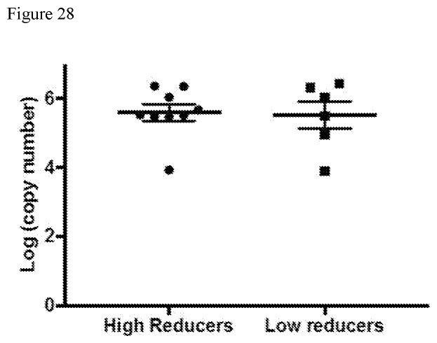

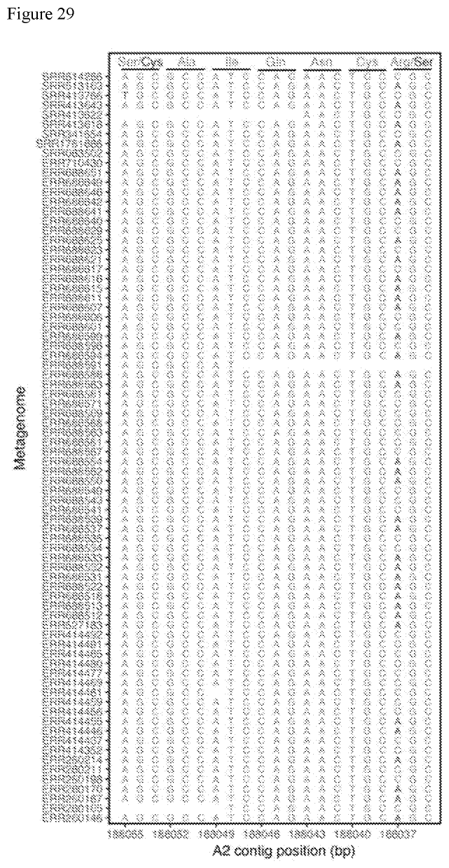

In contrast, as expected from previous experiments, neither the abundance of E. lenta nor dadh predicted dopamine dehydroxylation in complex gut microbial communities (FIG. 3E and F and FIG. 28). However, when dadh was amplified from these cultures and determined the SNP status at position 506, it was found that samples containing the Arg506 variant quantitatively metabolized dopamine, while the activity of samples carrying the Ser506 variant was indistinguishable from the non-metabolizing E. lenta DSM2243 strain (FIG. 3, Panel G). These findings indicate a single amino acid residue in a gut microbial enzyme predicts dopamine metabolism in complex communities. Given that dadh is highly prevalent (>70%) in gut microbiomes from human subjects and the two dadh variants are present among this population (FIGS. 26 and 29), it was speculated that SNPs may influence xenobiotic metabolism in the context of both the host genome and the human gut microbiome.

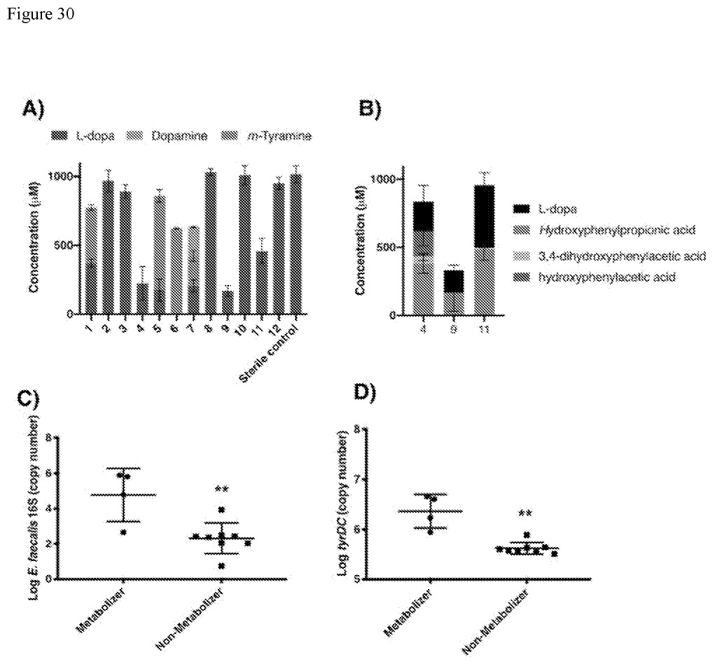

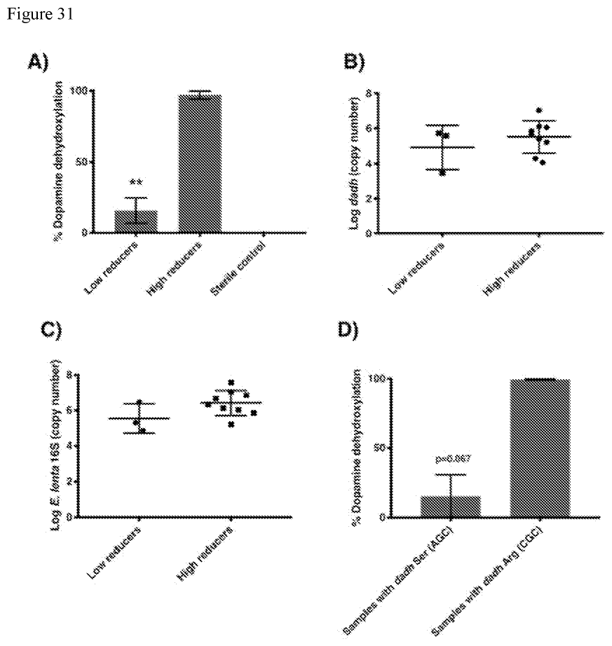

To further explore the clinical relevance of these findings, the metabolism of dopamine and L-dopa by fecal suspensions was assessed from Parkinson's disease patients ex vivo. Similar to control subjects, these individuals displayed significant variability in metabolism of L-dopa (FIG. 30, Panel A). qPCR assays revealed that tyrDC abundance and E. faecalis abundance discriminated between L-dopa decarboxylating and non-decarboxylating samples (p<0.005, one-tailed Mann-Whitney test) (FIG. 30, Panel C and D). Depletion of L-dopa was observed without corresponding production of dopamine or m-tyramine in three samples (FIG. 30, Panel A). Instead, L-dopa was converted to hydroxyphenylpropionic acid (hPPA) (FIG. 30, Panel B), a pathway thought to make a minor contribution to drug metabolism in vivo. Finally, it was found that the dadh SNP predicted dopamine dehydroxylation in these samples (FIG. 31). Overall, these data support a role for gut bacteria in the extensive inter-individual variability in L-dopa decarboxylation observed in Parkinson's patients. A recent study reported that stool tyrDC abundance is positively correlated with L-dopa dosage in patients but did not demonstrate a connection between tyrDC and L-dopa decarboxylation in these samples. These findings indicate this metabolic activity may indeed affect L-dopa therapeutic efficacy.

### (S)-α-Fluoromethyltyrosine (AFMT) Inhibits Gut Microbial L-Dopa Metabolism

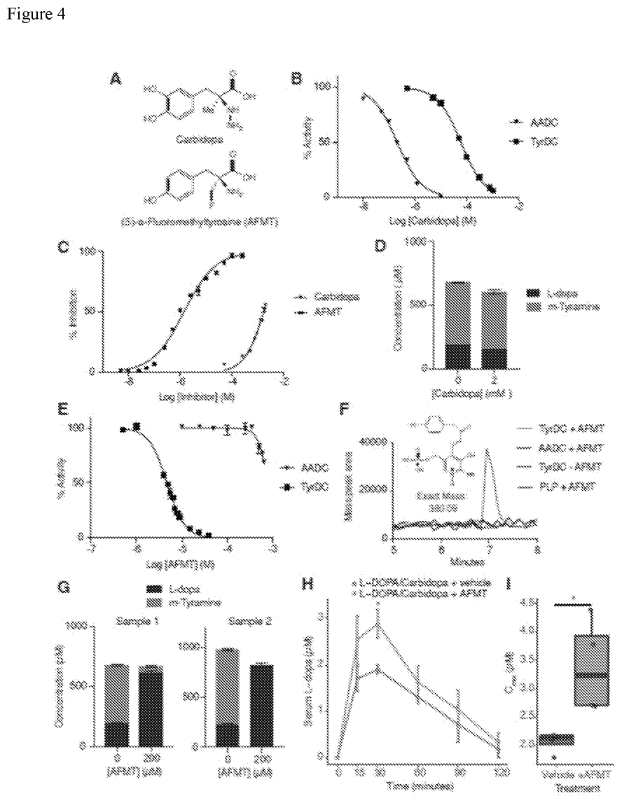

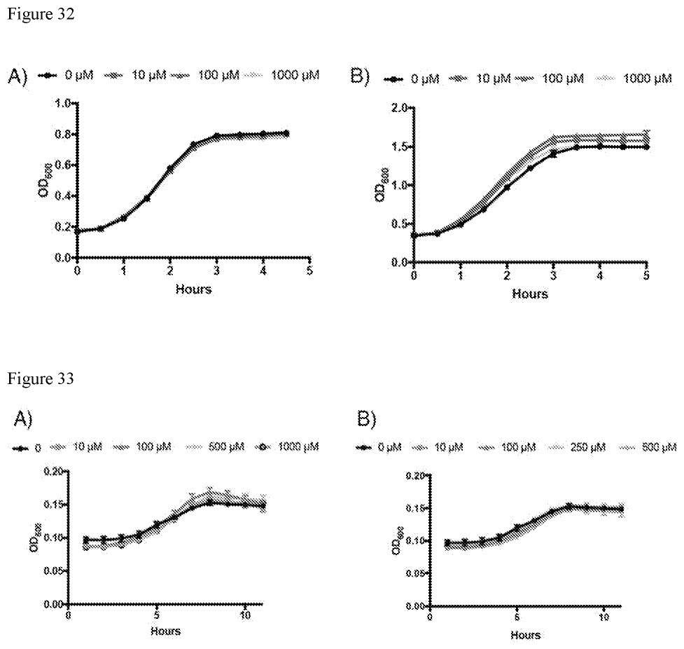

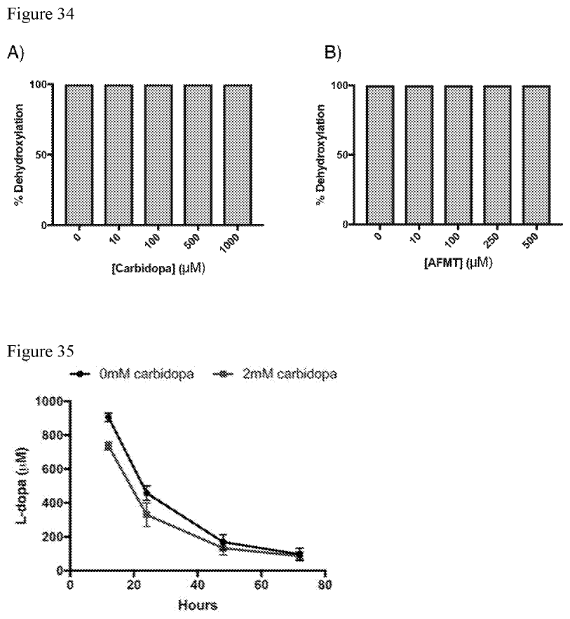

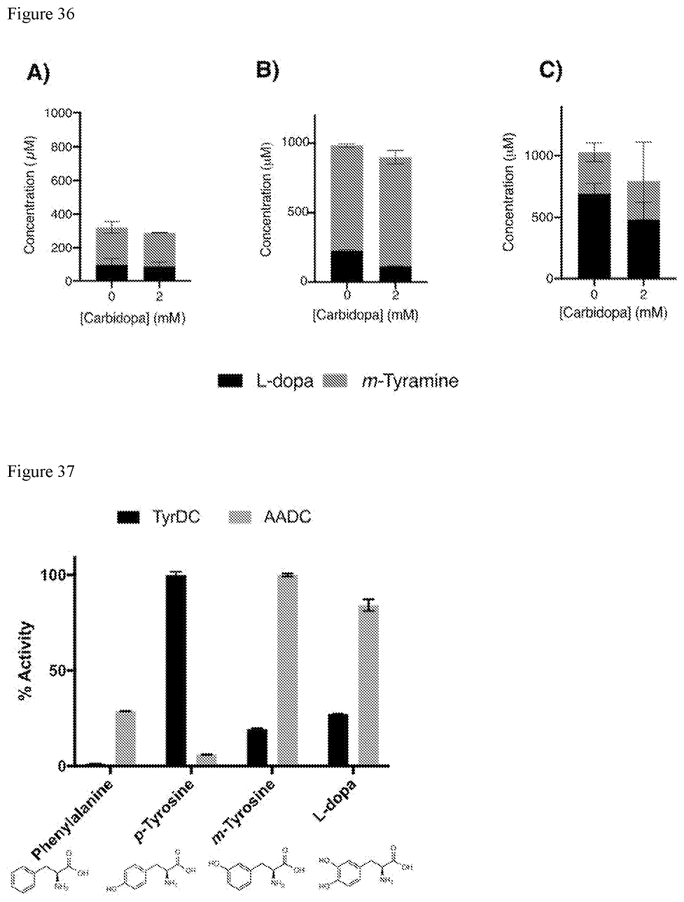

Having shown that E. faecalis and E. lenta enzymes predict L-dopa metabolism by complex patient gut microbiotas, it was next investigated whether this interspecies pathway was susceptible to inhibition by drugs that target peripheral L-dopa decarboxylation. In the United States, Parkinson's patients are co-prescribed carbidopa (FIG. 4, Panel A), an L-dopa mimic that inhibits AADC by forming a stable, covalent hydrazone linkage with its PLP cofactor. It was found that carbidopa was 200-fold less active toward purified E. faecalis TyrDC (IC₅₀=57 μM) relative to H. sapiens AADC (IC₅₀=0.21 μM) and showed only ˜50% inhibition of L-dopa decarboxylation by E. faecalis cultures at the solubility limit of 2 mM (FIG. 4, Part B and C, table 8), which is consistent with recently reported findings. Additionally, carbidopa did not affect growth of E. faecalis or metabolism or growth of E. lenta (FIGS. 32 to 34). Given the maximum predicted gastrointestinal concentration of carbidopa (0.4-9 mM), these data suggest this drug does not fully inhibit gut bacterial L-dopa decarboxylation in Parkinson's patients. Indeed, it was found that 2 mM carbidopa did not alter the kinetics of L-dopa degradation (FIG. 35) or endpoint m-tyramine production in stool samples from both Parkinson's patients and neurologically healthy controls (FIG. 4, Part D and FIG. 36). These observations support previous findings that carbidopa administration does not impact m-tyramine production in patients.

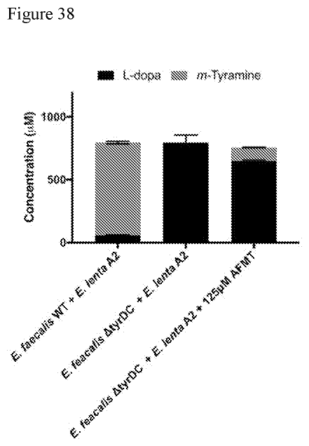

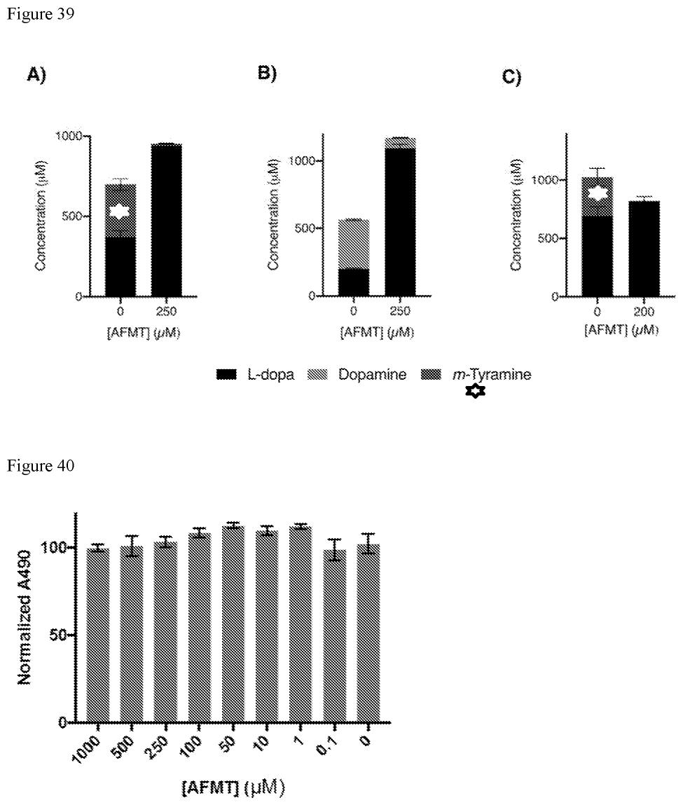

To selectively manipulate gut bacterial TyrDC in complex microbiotas, α-fluoromethyl amino acids were tested, which are known mechanism-based inhibitors of PLP-dependent decarboxylases. A survey of potential amino acid substrates revealed that TyrDC requires a p-hydroxyl group for robust activity while AADC prefers a m-hydroxyl substituent (FIG. 37), leading us to hypothesize the L-tyrosine analog (S)-α-fluoromethyltyrosine (AFMT) (FIG. 4, Panel A) might selectively inhibit the microbial enzyme. In vitro, AFMT strongly inhibited L-dopa decarboxylation by TyrDC (IC₅₀=4.7 μM) but not AADC (˜20% inhibition at solubility limit of 650 μM) (FIG. 4E and table 8). Consistent with this selectivity, AFMT formed a covalent PLP adduct only in the presence of TyrDC (FIG. 4, Panel F) AFMT was also effective in E. faecalis cultures (EC₅₀=1.4 μM) (FIG. 4, Panel C), outperforming carbidopa by 1000-fold without affecting growth (table 8 and FIG. 33). It also reduced L-dopa decarboxylation by co-cultures of E. faecalis and E. lenta without impacting growth or metabolism of E. lenta (FIGS. 33, 34, and 38). Finally, AFMT completely inhibited L-dopa decarboxylation in gut microbiota samples from Parkinson's disease patients and neurologically healthy control subjects (FIG. 4, Panel G and FIG. 39) and was non-toxic to eukaryotic cells (FIG. 40).

To investigate AFMT activity in vivo, either AFMT (25 mg/kg) or a vehicle control were administered in combination with L-dopa (10 mg/kg) and carbidopa (30 mg/kg) to gnotobiotic mice colonized with E. faecalis MMH594 (FIG. 4, Panel H). It was found that AFMT significantly increased the peak serum concentration (Cₘₐₓ) of L-dopa compared to vehicle (FIG. 4, Panel I) (p<0.05, two-tailed Mann Whitney test), which is consistent with inhibition of first-pass gut microbial metabolism in the intestine. Though it cannot be ruled out the possibility that AFMT modulates additional, uncharacterized targets, this observation is consistent with in vitro inhibition data. This result also aligns with a recent report that small intestinal tyrDC abundance or treatment with an E. faecalis tyrDC mutant strain negatively correlates with serum L-dopa levels in conventional rats receiving L-dopa and carbidopa and L-dopa.

### Materials and Methods

### General Materials

The following chemicals were used in this study: L-dopa (Sigma-Aldrich, catalog # D9628-5G), dopamine (Sigma-Aldrich, catalog # PHR1090-1G), m-tyramine (Santa Cruz Biotechnology, catalog # sc-255257), Isopropyl β-D-1-thiogalactopyranoside (Sigma-Aldrich, catalog # I5502-1G), carbidopa (Sigma-Aldrich, catalog # PHR1655-1G), L-tyrosine (Sigma-Aldrich, catalog # T3754-50G), L-arginine (Sigma-Aldrich, catalog # A5006-100G), sodium molybdate (Sigma-Aldrich, catalog #243655-10OG), Sodium tungstate (72069-25G), Pyridoxal-L-Phosphate (Sigma-Aldrich, catalog # P9255-1G), SIGMAFAST protease inhibitor tablets (Sigma-Aldrich, catalog #: S8830), (S)-α-fluoromethyltyrosine (AFMT) (obtained from Merck Sharp & Dohme Corp under MTA LKR166502), L-phenylalanine (Sigma-Aldrich, catalog # P2126-100G), d₃-phenyl-L-dopa (Sigma-Aldrich, catalog #333786-250 MG), benzyl viologen (Sigma-Aldrich, catalog # 271845-250mg), methyl viologen (Sigma-Aldrich, catalog #856177-1g), diquat (Sigma-Aldrich, catalog #45422-250 mg), sodium dithionite (Sigma-Aldrich, catalog #157953-5G). Luria-Bertani (LB) medium was prepared from its basic components (10 g/L tryptone, 5 g/L yeast extract, 5 g/L NaCl) or obtained from either EMD Millipore or Alfa Aesar. Acetonitrile and methanol for LC-MS analyses were purchased as LC-MS grade solvent from Honeywell Burdick & Jackson or Sigma-Aldrich.

### General Methods

All bacterial culturing work was performed in an anaerobic chamber (Coy Laboratory Products) under an atmosphere of 10% hydrogen, 10% carbon dioxide, and nitrogen as the balance. Hungate tubes were used for anaerobic culturing unless otherwise noted (Chemglass, catalog # CLS-4209-01). All lysate work and protein experiments were performed in an anaerobic chamber (Coy Laboratory Products) under an atmosphere of 10% hydrogen and nitrogen as the balance situated in a cold room at 4° C.

All genomic DNA (gDNA) was extracted from bacterial cultures using the DNeasy UltraClean Microbial Kit (Qiagen, catalog #: 12224-50) according to the manufacturer's protocol.

All cloning work was performed as follows. Ncol (catalog # R3193 S), HindIII (catalog # R0104S), XhoI (catalog # R0146s) and NdeI (R0111L) were purchased from New England Biolabs. For restriction digestion, 500 ng plasmid or 50 ng PCR insert were mixed with 1 μL of each restriction enzyme and 4μL 10× cutsmart buffer (New England Biolabs, catalog # B7200S), and MilliQ water to a final reaction volume of 40 μL. Restriction digestion reactions were left at 37° C. for 3 hours, followed by gel purification using the GFX PCR DNA and Gel Band Purification Kit (GE healthcare, catalog #28-9034-70). Ligation of purified digested vectors and inserts was performed by Gibson Assembly. Briefly, 50 ng of vector was mixed with 3-fold molar excess of insert, 5 μL Gibson Assembly 2× Mastermix (New England Biolabs, catalog # E2611S), and MilliQ water to a final volume of 10 μL. The Gibson reactions were left at 50° C. for 30 minutes and 5 μL of the reaction was transformed into chemically-competent E. coli TOP10 using heat shock.

### LC-MS Methods

Method A: Samples were analyzed using an Agilent technologies 6410 Triple Quad LC/MS and a Dikma Technologies Inspire PFP column (4.6×100 mm, 3.5 μm; catalog #81601). The flow rate was 1.0 mL min⁻¹using 0.1% formic acid in water as mobile phase A and 0.1% formic acid in acetonitrile as mobile phase B. The column temperature was maintained at room temperature. The following gradient was applied: 0-2 min: 0% B isocratic, 2-9 min: 0-10% B, 9-11 min: 10-95% B, 11-13 min: 95% B isocratic, 13-15 min: 95-0% B, 15-18 min: 0% B isocratic. For mass spectrometry, the source temperature was 300° C., and the masses of d₃-phenyl-L-dopa (precursor ion m/z=201.3, daughter ion m/z=155.2), L-dopa (precursor ion m/z=198.3, daughter ion m/z=152.2), d₃-phenyl-dopamine (precursor ion m/z=157.3, daughter ion m/z=140.3), dopamine (precursor ion m/z=154.3, daughter ion m/z=137.3), d₃-phenyl-tyramine (precursor ion m/z=141.3, daughter ion m/z=124.3), tyramine (precursor ion m/z=138.3, daughter ion m/z=121.3) were monitored at a collision energy of 15 mV and fragmentor setting of 135 in MRM mode.

Method B: Samples were analyzed using an Agilent technologies 6410 Triple Quad LC/MS and a Phenomenex Kinetex 5 μm Biphenyl 100 Å (50*4.6 mm, product #: 00B-4627-E0). The flow rate was 0.4 mL min⁻¹ using 0.1% formic acid in water as mobile phase A and methanol as mobile phase B. The column temperature was maintained at room temperature. The following gradient was applied: 0-6 min: 0% B isocratic. The same masses as in Method A were monitored using the same settings.

Method C: Samples were analyzed using an Agilent technologies 6410 Triple Quad LC/MS and a Thermo Scientific Acclaim polar advantage II (3 μM, 120 Å, 2.1*150 mm, product #: 063187). The flow rate was 0.2 mL min⁻¹ using 0.1% formic acid in water as mobile phase A and methanol as mobile phase B. The following gradient was applied: 0-4 min: 50% B isocratic, 4-7 min: 50-99%, 7-9 min: 99-50%, 9-13 min: 50% B isocratic. The same masses as in Method A were monitored using the same settings.

Method D: Identical to Method A, but an additional mass of phenylethylamine (precursor ion m/z=122.3, daughter ion m/z=105.3) was monitored at a collision energy of 15 mV and fragmentor setting of 135 in MRM mode.

Method E: Samples were analyzed using an Agilent technologies 6530 Accurate-Mass Q-TOF LC/MS and a Phenomenex Kinetex 5 μm Biphenyl 100 Å (50*4.6 mm, product #: 00B-4627-E0). The flow rate was 0.4 mL min⁻¹ using 0.1% formic acid in water as mobile phase A and 0.1% formic acid in acetonitrile as mobile phase B. The column temperature was maintained at room temperature. The following gradient was applied: 0-2 min: 5% B isocratic, 2-25 min: 0-95% B, 25-30 min: 95% B isocratic, 30-40 min: 95-5% B. For the MS detection, the ESI mass spectra data were recorded on a positive mode for a mass range of m/z 50 to 3000. A mass window of±0.005 Da was used to extract the ion of [M+H].

Method F: Identical to Method A, but an additional mass of tyrosine (precursor ion m/z=182.3, daughter ion m/z=136.2) was monitored at a collision energy of 15 mV and fragmentor setting of 135 in MRM mode.

Method G: Samples were analyzed using an Agilent technologies 6410 Triple Quad LC/MS and a Thermo Scientific Acclaim polar advantage II (3 120 Å, 2.1*150 mm, product #: 063187). The flow rate was 0.2 mL min⁻¹ using 0.1% formic acid in water as mobile phase A and methanol as mobile phase B. The following gradient was applied: 0-4 min: 50% B isocratic, 4-7 min: 50-99%, 7-8 min: 99% C isocratic, 8-9 min: 99-50% B, 9-13 min: 50% B isocratic. For mass spectrometry, the source temperature was 300° C., and the masses of d₃-phenyl-dihydrocaffeic acid (precursor ion m/z=184.2, daughter ion m/z=140.2), d₃-phenyl-dihydroxyphenylacetic acid (precursor ion m/z=170.2, daughter ion m/z=126.2), d₃-phenyl-hydroxyphenylpropionic acid (precursor ion m/z=168.2, daughter ion m/z=124.2), d₃-phenyl-hydroxyphenylacetic acid (precursor ion m/z=154.2, daughter ion m/z=110.2) were monitored at a collision energy of 15 mV and fragmentor setting of 135 in MRM mode.

Method H: Samples were analyzed using an Agilent technologies 6410 Triple Quad LC/MS and a Dikma Technologies Inspire Phenyl column (4.6×150 mm, 5 μm; catalog #81801). The flow rate was 0.5 mL min⁻¹ using 0.1% formic acid in water as mobile phase A and 0.1% formic acid in acetonitrile as mobile phase B. The column temperature was maintained at room temperature. The following gradient was applied: 0-9 min: 0-10% B, 9-11 min: 10-95% B, 11-13 min: 95% B isocratic, 13-14 min: 95-0% B, 14-18 min: 0% B isocratic. For mass spectrometry, the source temperature was 300° C., and the masses of d₃-phenyl-L-dopa (precursor ion m/z=201.3, daughter ion m/z=155.2), L-dopa (precursor ion m/z=198.3, daughter ion m/z=152.2), d₃-phenyl-dopamine (precursor ion m/z=157.3, daughter ion m/z=140.3), dopamine (precursor ion m/z=154.3, daughter ion m/z=137.3), d₃-phenyl-tyramine (precursor ion m/z=141.3, daughter ion m/z=124.3), tyramine (precursor ion m/z=138.3, daughter ion m/z=121.3) were monitored at a collision energy of 15mV and fragmentor setting of 135 in MRM mode.

### pBLAST of L. brevis TyrDC Among Human Microbiome Project Reference Isolates

The L. brevis tyrosine decarboxylase (UniProt accession, B8V35) was used as the query sequence for a BLASTP search of the Human Microbiome Project (HMP) reference isolate genomes. All GenBank assemblies associated with the Human Microbiome Project Reference Genome project (PRJNA28331) were retrieved (20 Jun. 2018). Protein sequences were collected were queried by BLASTP (BLAST+2.6.0) requiring a minimum e-value of 1e−4. Separately, scaffolds were queried for 16S rRNA genes (github.com/tseemann/barrnap). Where multiple copies were present, the longest was selected, and if multiple equal length copies were present, the first was selected. An alignment was carried out using muscle with the following parameters: ‘maxiters 3-diags1-sv’. The alignment was trimmed removing positions where >20% of alignment was gapped and a tree was built using FastTree. The tree was subsequently rooted on Methanobrevibacterial species and represented as a cladogram with ggtree.

### Assessment of Conservation of TyrDC Across E. faecalis Genomes

The amino acid sequence of TyrDC from E. faecalis TX0645 (UniProtKB #E6I994) was subjected to a tBLASTn search against whole genome shotgun contigs in NCBI using the default BLAST parameters. All but 3 of the 655 E. faecalis genomes that were searched had the TyrDC sequence conserved at 98% amino acid ID and 100% query cover.

### Screen for L-Dopa Decarboxylation in Anaerobic Bacterial Cultures

The strains screened for decarboxylation of L-dopa were Enterococcus faecalis MMH549, Enterococcus faecalis V583, Enterococcus faecalis OG1RF, Enterococcus faecalis TX0104, Enterococcus faecium E1007, Enterococcus faecium E2134, Enterococcus faecium TX01330, Providencia rettgeri DSM 1131, Proteus mirabilis ATCC 29906, Lactobacillus brevis subsp. gravesensis ATCC 27305. E. faecalis and E. faecium were grown in Brain Heart Infusion (BHI) broth (Beckton Dickinson, catalog #211060), while P. rettgeri and P. mirabilis were grown in MEGA medium. L. brevis was grown in MRS medium (Sigma-Aldrich, catalog #69966-500G). Starter cultures were grown from individual colonies for 48 hours at 37° C. in liquid media and were then inoculated 1:100 into triplicate tubes containing fresh media and 500 μM L-dopa. Cultures were grown for 48 hours at 37° C., after which they were harvested by centrifugation. Culture supernatant was diluted 1:20 in LC-MS grade MeOH, and 5 μL of this supernatant was analyzed on LC-MS/MS using Method A described above. % Decarboxylation was calculated as the concentration of dopamine divided by the total concentration of dopamine and L-dopa in the culture. All experiments were performed in an anaerobic chamber.

### Confirmation of E. faecalis MMH594 tyrDC Mutant

Mutants were generated as part of a transposon mutagenesis library and were generously provided by the Gilmore lab at Massachusetts General Hospital. The 2 kb Tet cassettes were verified by PCR using the primer pair ATGAAAAACGAAAAATTAGCAAAAGG and CATACCATAAGCCTTCTAAGTTAGC.

### Cloning and Expression of E. faecalis MMH594 Tyrosine Decarboxylase

AtyrDC was amplified from E. faecalis MMH594 genomic DNA with primers TGGTGCCGCGCGGCAGCCATATGAAAAACGAAAAATTAGCAAAAG and TGGTGGTGGTGGTGCTCGAGTTATTTTACGTCGTAAATTTGTTC. The purified PCR product and the pET-28a vector were digested with XhoI and NdeI and, following purification of the digested vector, were ligated together using Gibson assembly. The final vector Pet-28a encoding N-His₆ TyrDC was confirmed to contain the appropriate insert by Sanger sequencing. For protein expression, the pET-28a N-His₆ TyrDC vector was transformed into chemically competent E. coli BL21(DE3). Starter cultures were grown from individual colonies in 5 mL of LB medium containing 50 μg/mL kanamycin for 18 hours and were then inoculated into 500 mL of LB medium containing 50 μg/mL kanamycin. Cells were grown with shaking at 37° C. until reaching OD₆₀₀=0.400, at which point isopropyl β-D-1-thiogalactopyranoside (IPTG) was added to a final concentration of 200 μM. Cells were grown for 18 hours at 15° C. with shaking. For purification of recombinant TyrDC, the 500 mL culture was harvested by centrifugation, and the resulting pellet was resuspended in 40 mL 50 mM Tris pH containing 0.25M NaCl, followed by lysis using a cell disruptor (Avestin emulsiflex C3). All of the clarified lysate was loaded onto 2 mL of HisPur Ni-NTA resin (Thermo Fisher Scientific, Waltham, Mass.) and eluted using a gradient of 50 mM to 200 mM imidazole (in 50 mM Tris pH 8 containing 0.25M NaCl). Fractions containing pure protein were combined and dialyzed over two rounds into in 50 mM Tris pH 8 containing 0.20 M NaCl and 10% w/v glycerol. The dialyzed protein was concentrated 10-fold using spin columns (VMR, catalog #97027-9) and frozen at a final concentration of ˜100 μM in liquid nitrogen. Protein aliquots were stored at −80° C.

### Michaelis-Menten Kinetics of E. faecalis TyrDC

To assess the steady-state kinetics of TyrDC towards tyrosine and L-dopa, the enzyme was first pre-incubated with pyridoxal-5′-phosphate (PLP) at a ratio of 1:1333 in 0.2 M sodium acetate buffer, pH 5.5 for five minutes. The enzyme reaction was then initiated by dissolving the preincubated enzyme-PLP mix 1:10 with substrate dissolved in 0.2 M sodium acetate buffer, pH 5.5. The final concentrations in the final enzyme reaction were 200 μM PLP, 0.15 μM enzyme and 100 μM-1800 μM tyrosine or 100 μM-2250 μM L-dopa. Aliquots of the enzyme reaction were quenched by diluting 10-fold into ice cold methanol at 20, 40, and 60 seconds after initiation. The quenched reaction was centrifuged to pellet precipitates and 5 μL of the supernatant was analyzed by LC-MS/MS using Method B described above. Rates were calculated as the substrate produced from 20 to 60 seconds and were fit to a standard Michaelis-Menten kinetics curve in Graphpad prism (version 7). All experiments were done in triplicate and were repeated at least twice. Reactions were performed at room temperature.

### Competition of L-Dopa and Tyrosine for Reaction With Purified E. faecalis TyrDC

The buffers described above were used for each enzyme in this assay. 0.15 TyrDC was mixed with 200 μM PLP and equimolar concentrations of L-dopa and tyrosine (both at a final concentration of 500 Formation of the corresponding amine products was measured by LC-MS/MS following quenching with ice cold methanol (1:10) at specific time points. The quenched mixture was then centrifuged for 10 minutes to pellet precipitates. Method D was used for LC-MS/MS analysis of the supernatant as described above. Reactions were performed at room temperature.

### Assessment of the Impact of pH on L-Dopa and Tyrosine Decarboxylation by E. faecalis

E. faecalis MMH594, E. faecalis V583, E. faecalis TX0104, E. faecalis OG1RF were inoculated from single colonies into 10 mL of BHI medium in individual Hungate tubes. Following 24 hours of anaerobic growth, these starter cultures were inoculated in triplicate 1:10 into 200 μL of BHI medium (pH 5 or pH 7) and were grown at 37° C. anaerobically in 96-well plates (VWR, catalog #29442-054). Plates were set up in duplicate. One of these plates was used to measured growth in the Synergy HTX Multi-Mode Microplate Reader (BioTek) by measuring absorbance at 600 nm. The other plate was used for withdrawing culture aliquots for metabolite analysis; at 0, 2, 4, 8, 24 hours, a 30 μL aliquot was removed from each culture and immediately frozen at −20° C. for downstream metabolite analysis. Thawed aliquots were centrifuged to pellet the cells and supernatants were diluted 1:10 in LC-MS grade methanol and were analyzed by LC-MS using Method F described above.

### Assessment of the Impact of pH and Tyrosine on L-Dopa Decarboxylation by E. faecalis MMH594

BHI medium was adjusted to the appropriate pH with HCl prior to autoclaving. E. faecalis MMH594 was inoculated from single colonies into 10 mL of BHI medium in individual Hungate tubes. Following 24 hours of anaerobic growth, these starter cultures were inoculated in triplicate 1:10 into 200 μL of BHI medium (pH 5 or pH 7) containing either 0 or 1 mM L-tyrosine added. The final tyrosine concentrations in the BHI medium were approximately 500 μM without added tyrosine and 1500 μM with tyrosine added. These two concentrations approximate the resting and post-meal small intestinal tyrosine concentration in healthy human volunteers. The cultures were grown at 37° C. anaerobically in 96-well plates (VWR, catalog #29442-054). Plates were set up in duplicate. One of these plates was used to measured growth in the Synergy HTX Multi-Mode Microplate Reader (BioTek) by measuring absorbance at 600 nm. The other plate was used for withdrawing culture aliquots for metabolite analysis; at 0, 2, 4, 8, 24 hours, a 30 μL aliquot was removed from each culture and immediately frozen at −20° C. for downstream metabolite analysis. Thawed aliquots were centrifuged to pellet the cells and supernatants were diluted 1:10 in LC-MS grade methanol and were analyzed by LC-MS using Method F described above.

### Assessment of the Impact of pH and Tyrosine on L-Dopa Decarboxylation by Three Fecal Samples From Neurologically Healthy Humans

BHI medium was adjusted to the appropriate pH using HCl prior to autoclaving. Fecal slurries had been previously frozen in PBS with 20% glycerol. These samples were thawed at room temperature anaerobically and inoculated 1:200 into 5 mL BHI medium (pH 5 or pH 7) with either 0 or 1 mM L-tyrosine added. The final tyrosine concentrations in the BHI medium were approximately 500 μM without added tyrosine and 1500 μM with tyrosine added. These two concentrations approximate the resting and post-meal small intestinal tyrosine concentration in healthy human volunteers. Experiments were performed anaerobically, and cultures were grown in Hungate tubes at 37° C. At 0, 11, 23, 32, and 48 hours, a 200 μL aliquot was removed from each culture. Following measurement of absorbance at 600 nm using a Synergy HTX Multi-Mode the Microplate Reader (BioTek), aliquots were immediately frozen at −20° C. for downstream metabolite analysis. The thawed aliquots were centrifuged to pellet the cells, and supernatants were diluted 1:10 in LC-MS grade methanol and were analyzed by LC-MS using Method F described above.

### Calculation of Drug Concentrations in the Small Intestine

The maximum molar concentration (molarity) likely to be achieved by a given drug was calculated by converting the dose amount into moles and then dividing by 100 mL, the approximate volume of the small intestine after drinking 240 mL of water. For levodopa, the dose range considered was 0.10 to 6.0 grams, which resulted in a range of 0.51 to 30.4 mmol and thus a concentration range of 5.1 to 304 mM. For carbidopa, the dose range considered was 10 mg to 200 mg (16), which resulted in a range of 0.044 to 0.88 mmol and thus a concentration range of 0.44 to 88 mM.

### Cloning and Heterologous Expression of H. sapiens Aromatic Amino Acid Decarboxylase

H. sapiens aromatic amino acid decarboxylase (AADC) was obtained as cDNA from Sino Biological (catalog #: HG10560-M). The cDNA was amplified using primers AACCATGGATGAACGCAAGTGAATTCCGAAGG and GGAAGCTTCTCCCTCTCTGCTCGCAGC. PCR amplicons were purified using the Illustra GFX PCR DNA and Gel Band Purification Kit (GE healthcare, catalog #28-9034-70) The purified PCR amplicon as well as Pet-28a were digested by NcoI and HindIII restriction enzymes using standard protocols. The insert was ligated into pET-28a using Gibson assembly. The final pET-28a vector encoding C-His₆ AADC was confirmed to contain the appropriate insert by Sanger sequencing. For protein expression, the pET-28a C-His₆ AADC vector was transformed into chemically competent E. coli BL21 (DE3). Starter cultures were grown from individual colonies in 5 mL of LB medium containing 50 μg/mL kanamycin for 18 hours and were then inoculated into 4 L of LB medium containing 50 μg/mL kanamycin. When OD₆₀₀ reached 0.500, IPTG was added at a final concentration of 200 μM to induce protein expression. Cells were grown for 18 hours at 15° C. with shaking. The purification of AADC followed the general protocol outlined by Montioli et al. with minor modifications. Briefly, cultures were harvested by centrifugation and the resulting pellet was resuspended in 40 mL 20 mM sodium phosphate buffer pH 7.4 containing 0.5 M NaCl, 20 mM imidazole, 4 mg/mL protease inhibitor tablets (Sigma-Aldrich, catalog #: S8830) and 50 μM PLP, followed by lysis using a cell disruptor (Avestine emulsiflex C3). All the clarified lysate was loaded onto Ni resin and eluted in a gradient from 50 mM to 200 mM imidazole (in 20 mM sodium phosphate buffer pH 7.4 containing 0.5 M NaCl). Pure fractions were combined and dialyzed over two rounds into in 100 mM sodium phosphate buffer pH 7.4 and 10% w/v glycerol. The dialyzed protein was concentrated 10-fold using spin columns and frozen at a final concentration of ˜80 μM in liquid nitrogen. Protein aliquots were stored at −80° C.

### Evaluation of Inhibitors Towards E. faecalis and H. sapiens Decarboxylases

To assess the inhibitory effects of carbidopa on L-dopa decarboxylation, the general process and setup described in was followed, with some modifications. For TyrDC, enzyme was first pre-incubated at a ratio of 1:1333 with PLP in 0.2 M sodium acetate buffer, pH 5.5 for five minutes. The enzyme reaction was then initiated by dissolving the preincubated enzyme-PLP mix 1:10 with substrate and inhibitor dissolved in 0.2 M sodium acetate buffer, pH 5.5. The final concentrations in the enzyme reaction were 200 μM PLP, 0.15 μM enzyme, 500 μM L-dopa and 0.5 μM-1000 μM carbidopa. Aliquots of the enzyme reaction were quenched by diluting 10-fold into methanol at 20, 40, and 60 seconds after initiation. For AADC, enzyme was pre-incubated for five minutes at a ratio of 1:500 with PLP in 20 mM sodium phosphate buffer, pH 7.4. The enzyme reaction was then initiated by dissolving the preincubated enzyme-PLP mix 1:10 with substrate and inhibitor dissolved in 20 mM sodium phosphate buffer, pH 7.4. The final concentrations in the enzyme reaction were 100 μM PLP, 0.2 μM enzyme, 500 μM L-dopa and 0.01 μM-10 μM of carbidopa. Aliquots of the enzyme reaction were quenched by diluting 10-fold into methanol at 60, 120, and 180 seconds after initiation. For (S)-α-fluoromethyltyrosine (AFMT), the same buffer conditions as above were used for each enzyme but following a slightly different setup. For TyrDC, 1 μM enzyme was first preincubated with 2000 μM PLP and AFMT (0.5-20 μM) for 14 minutes while for AADC, 1μM enzyme was first preincubated with 200 μM PLP and AFMT (10-650 μM), for 14 minutes. Reactions were initiated by diluting this mixture 1:10 in buffer containing 500 μM L-dopa. The TyrDC reaction was quenched by diluting 1:10 in MeOH after 60 seconds while the AADC reaction was quenched after 120 seconds. For both AADC and TyrDC, the quenched reactions were spun down, and 5 μL of the supernatant was analyzed by LC-MS/MS using Method B described above. IC₅₀ curves were fit in Graphpad Prism (version 7). All experiments were done in triplicate and were repeated at least twice. Rates were calculated as the substrate produced over the timepoints collected and were normalized to the rate without inhibitor to produce a measure of % activity. Reactions were performed at room temperature.

### Identification of a PLP-Inhibitor Adduct Upon Incubation of Enzymes With AFMT

The same buffers as described above were used for each enzyme. 10 μM AADC or TyrDC was incubated with 200 μM PLP and 50 μM AFMT for one hour at room temperature. The reactions were quenched by boiling at 95° C. for 15 minutes. This mix was diluted 1:20 in LC-MS grade methanol and spun down to pellet precipitates. The supernatant was analyzed by LC-MS using Method E described above. The predicted adduct was identified based on the work described in.

### Substrate Screen With E. faecalis TyrDC and H. sapiens AADC

The same buffers as described above were used for each enzyme. 0.15 μM TyrDC was mixed with 200 μM PLP, and 0.2 μM AADC was mixed with 200 μM PLP. L-dopa, phenylalanine, m-tyrosine, or p-tyrosine was added to each of the enzymes at a final concentration of 500 μM. The AADC reaction was quenched at 180 seconds and the TyrDC reaction was quenched at 60 seconds, both by diluting 1:10 in ice cold methanol. The plates were centrifuged to pellet precipitates and supernatants were analyzed on LC-MS/MS using Method D described above. Reactions were performed at room temperature.

### Evaluation of Inhibitors Towards Growth of E. faecalis and E. lenta

E. faecalis MMH594 starter cultures were grown in BHI medium anaerobically over 12 hours from individual colonies at 37° C., while E. lenta was grown for 48 hours in BHI medium. The starter culture was diluted 1:10 in 180 μL of fresh BHI medium containing 500 μM L-dopa or dopamine and varying concentrations of carbidopa or AFMT. Cultures were grown anaerobically at 37° C. for 18 hours and harvested by centrifugation. Supernatants were diluted 1:10 in LC-MS grade methanol and were analyzed by LC-MS using Method C described above. % Inhibition was calculated as the concentration of L-dopa remaining relative to that remaining in culture supernatants from the E. faecalis MMH594 tyrDC mutant grown under the same conditions. Growth was monitored using a Synergy HTX Multi-Mode Microplate Reader (BioTek) by measuring absorbance at 600 nm. Experiments were performed anaerobically, and cultures were grown in 96-well plates (VWR, catalog #29442-054).

### Screen of a Collection of Gut Bacteria in Anaerobic Culture for Dopamine Dihydroxylation Using a Colorimetric Assay

The colorimetric assay for dopamine dehydroxylation was based on the Arnow test (73). Briefly, 50 μL of 0.5 M HCl was added to 50 μL of culture supernatant. After mixing, 50 μL of an aqueous solution containing both sodium molybdate and sodium nitrite (0.1 g/mL each) was added, which produced a yellow color. Finally, 50 μL of 1 M NaOH was added followed by pipetting up and down to mix. This allowed the characteristic pink color to develop. Absorbance was measured at 500 nm immediately using a Synergy HTX Multi-Mode Microplate Reader (BioTek). Initially, a set of gut strains were grown in MEGA media anaerobically for 48 hours with 250 μM dopamine and used the colorimetric assay to assess their ability to dehydroxylate dopamine. None of the strains screened (Enterococcus faecalis MMH594, Enterococcus faecalis TX0104, Enterococcus faecium TX01330, Clostridium aspargiforme DSM15981, Flavonifractor plautii ATCC29863, Clostridium sp. ATCC BAA-442, Bifidobacterium longum spp. Infantis ATCC 15697, Flavonifractor plautii ATCC 49531, Clostridium ramosum DSM 1402, Akkermansia mucimphila ATCC BAA-835, Clostridium bartlettii AATCC BAA-827, Enterococcus raffinosus AATCC 49464, Eubacterium limosum AATCC 10825, Eggerthella lenta DSM2243, Faecalibacterium prausnitzii AATCC 27766, Eubacterium rectale AATCC 33656, Lactobacillus reuteri ATCC 23272, Parabacteroides distasonis AATCC 8503) displayed any detectable metabolism.

### Collection of Fecal Samples From Neurologically Healthy Human Patients

The indicated stool samples were collected from 12 neurologically healthy individuals during the control phase of an inpatient study described in detail elsewhere and from 7 healthy control subjects sampled at the University of California, San Francisco (UCSF). All subjects consented to participate in the study, which was approved by the relevant Institutional Review Boards.

### Collection of Fecal Samples From Parkinson's Disease Patients

The indicated stool samples were obtained from the BioCollective Microbiome Stool Bank (Denver, Colo.), which collected the samples under the Western IRB WIRB #1164096 for human subjects research. Samples were stored as fecal slurries in PBS and 20% glycerol at −80° C. until use. Specific samples (n=12) were selected from a larger set to minimize confounding variables, and they include 6 drug-naïve individuals and 6 patients taking L-dopa/carbidopa. Participants were not taking: antibiotics, antihistamines, laxatives, suppositories, beta blockers, statins, proton pump inhibitors, tricyclic antidepressents, selective serotonin reuptake inhibitors (SSRIs), platelet aggregators, oral contraceptives, oral metformin, and nonsteroidal anti-inflammatory drugs. They did not have additional comorbidities. Groups were balanced on gender (drug-naïve=3 males and 3 females vs. L-dopa/carbidopa=4 males and 2 females), age (drug-naïve=68±6 years vs. L-dopa/carbidopa=60±8 years), and body mass index (drug-naïve=23.1±4.4 kg/m² vs L-dopa/carbidopa=25.3±3.8 kg/m²) for drug naïve and L-dopa/carbidopa patients respectively (mean±sd).

### Enrichment Culturing and Isolation of a Dopamine Dehydroxylating Strain

To prepare the growth medium used for enrichment culturing, 10 g NaCl, 5 g MgCl₂*6H₂O, 2 g KH₂PO₄, 3 g NH₄Cl, 3 g KCl, 0.15 g CaCl₂×2H₂O, 1 g yeast extract, 1 g tryptone, 10 mL Trace Mineral Supplement (ATCC, catalog # MD-TMS), and 0.25 mL of 0.1% resazurin (dissolved in MilliQ water) were added to a final volume of 1 L of water. This medium was then boiled with stirring to dissolve all components. While cooling, the medium was bubbled with argon to ensure it was anaerobic. Once the medium was cool, 30 mM NaHCO₃ and 0.4 mM L-cysteine HCl were added, the medium was brought into an anaerobic chamber, and distributed in 10 mL aliquots into individual Hungate tubes. These tubes were autoclaved and brought back into an anaerobic chamber where 100 μL of Vitamin Supplement (ATCC, catalog # MD-VS) was added to each tube. The tubes were stored at 4° C.

During the enrichment culturing, all experiments were performed under strictly anaerobic conditions in an anaerobic chamber. First, a stool sample from a healthy human donor was resuspended in pre-reduced PBS at a final concentration of 0.1 g/mL. The mixture was vortexed to produce a homogenous slurry and was then left for 30 minutes to let particulates settle. The supernatant was diluted 1:10 in PBS and was further diluted 1:100 into the medium described above containing 500 μM dopamine as the electron acceptor and 10 mM sodium acetate as the electron donor. These initial cultures were incubatd at 37° C. for five days and were then passaged twice by a 1:100 dilution into fresh medium. Each successive passage was incubated for 48 hours. The final enrichment culture was streaked onto agar plates containing the basal medium described above. Once individual colonies appeared, they were picked and inoculated into the same liquid medium and the liquid cultures were screened for dopamine dehydroxylation after 48 hours of growth at 37° C. using the colorimetric assay described above. Colonies that displayed activity in the minimal medium were diluted 1:10 in PBS and plated onto agar plates containing MEGA Medium to support robust growth (68) at 37° C. for five days. Colonies that appeared on MEGA medium plates were then inoculated into liquid MEGA medium, and active colonies were re-streaked twice to ensure pure cultures. Throughout the experiment, dopamine dehydroxylation was tracked using the colorimetric assay for catechol detection. In addition, gDNA was harvested from cultures at different stages of enrichment using the DNeasy UltraClean Microbial Kit, allowing for 16S rRNA gene sequencing to be performed.

16S rRNA gene libraries targeting the V4 region of the 16S rRNA gene were prepared by first normalizing template concentrations and determining optimal cycle number by way of qPCR. Two 25 μL reactions for each sample were amplified with 0.5 units of Phusion polymerase with 1× High Fidelity buffer, 200 μM of each dNTP, 0.3 μM of 515F (5′-AATGATACGGCGACCACCGAGATCTACACTATGGTAATTGTGTGCCAGCMGCC GCGGTAA-3′) and 806rcbc0 (5′-CAAGCAGAAGACGGCATACGAGATTCCCTTGTCTCCAGTCAGTCAGCCGGACT ACHVGGGTWTCTAAT-3′). 0.25 μL of 100× SYBR were added to each reaction mixture, and samples were quantified using the formula 1.75\hat{ }(deltaCt). To ensure minimal over-amplification, each sample was diluted to the lowest concentration sample, amplifying with this sample optimal cycle number for the library construction PCR. Four 25 μL reactions were prepared per sample with master mix conditions listed above, without SYBR. Each sample was given a unique reverse barcode primer from the Golay primer set (Caporaso, 2011 and 2012). Replicates were then pooled and cleaned via Agencourt AMPure XP-PCR purification system. Purified libraries were diluted 1:100 and quantified again via qPCR (Two 25 μL reactions, 2x iQ SYBR SUPERMix (Bio-Rad, REF: 1708880 with Read 1 (5′-TATGGTAATT GT GTGYCAGCMGCCGCGGTAA-3′), Read 2 (5′-AGTCAGTCAGCCGGACTACNVGGGTWTCTAAT-3′)). Undiluted samples were normalized by way of pooling using the formula mentioned above. Pools were quantified by Qubit (Life Technologies, Inc.) and normalized into a final pool by Qubit concentration and number of samples. Final pools were sequenced on an Illumina MiSeq 300 using custom index 5′-ATTAGAWACCCBDGTAGTCCGGCTGACTGACT-3′ and custom Read 1 and Read 2 mentioned above. Sequencing data were analyzed using QIIME.

Eggerthella lenta was identified A2 as an active dopamine dehydroxylating strain. The sequencing of its genome has been previously described and this sequence has been deposited into NCBI.

### Screen of Gut Actinobacterial Isolates for Dopamine Dehydroxylation

Cells were cultured in 96-well plates and all experiments were performed anaerobically. The strains screened for dopamine dehydroxylation have been previously described. Individual strains were plated from glycerol stocks onto BHI agar plates containing 1% arginine and grown for 3 days. Colonies were then inoculated into BHI liquid medium and grown for 48 hours at 37° C. to provide turbid starter cultures, which were diluted 1:100 in triplicate into 200 μL fresh BHI medium containing 500 μM dopamine. These cultures were grown for 48 hours at 37° C. Cultures were harvested by centrifugation and the supernatant was diluted 1:10 with LC-MS grade methanol and analyzed by LC-MS/MS using Method C described above. The experiment was repeated twice.

### Lysate Assays to Assess Dopamine Dehydroxylation in E. lenta A2

10 mL turbid 48-hour starter cultures of E. lenta A2 in BHI medium were diluted 1:100 into 20 mL BHI medium containing 1% arginine and 10 mM formate with or without 500 μM dopamine. Cells were pelleted by centrifugation when cultures reached OD₆₀₀=0.500. Cell pellets were washed twice with PBS and were then re-suspended in 800 μL 50 mM Tris pH 8 containing 4 mg/mL SIGMAFAST protease inhibitor cocktail, followed by anaerobic sonication to lyse the cells. Dopamine was added to crude lysates at a final concentration of 500 μM and the reactions were left for 12 hours at room temperature under anaerobic conditions. To assess the impact of oxygen on dopamine dehydroxylation by cell lysates, the assay was also set up outside the anaerobic chamber. To assess the impact of tungstate on dopamine dehydroxylation by cell lysates, sodium tungstate (2.5, 5, and 10 mM) was added as a solid at the time of dopamine addition. After incubation, all lysates were dissolved 1:10 with LC-MS grade methanol and centrifuged to pellet precipitates. Supernatant were analyzed by LC-MS/MS to measure dopamine and m-tyramine production using Method A described above. Experiments were performed in triplicate and repeated twice to ensure consistency.

### RNA-sequencing in E. lenta A2, E. lenta DSM2243, and E. lenta 28b

Turbid 48-hour starter cultures of E. lenta in BHI medium were inoculated 1:100 into 5 mL BHI medium containing 1% arginine and 10 mM formate, and cultures were grown at 37° C. anaerobically. When cultures reached OD₆₀₀=0.200, dopamine or vehicle (water) was added at a final concentration of 500 μM to triplicate or quadriplicate cultures. Cultures were harvested when they reached OD₆₀₀=0.500. They were centrifuged, and cell pellets were re-suspended in 500 μL Trizol reagent (ThermoFisher, catalog #: 15596026). Total RNA was isolated first by bead beating to lyse cells and then using the Zymo Research Direct-Zol RNA MiniPrep Plus kit (Catalog # R2070) according to the manufacturer's protocol. Illumina cDNA libraries were generated using a modified version of the RNAtag-Seq protocol as described. Briefly, 500 ng of total RNA was fragmented, depleted of genomic DNA, and dephosphorylated prior to its ligation to DNA adapters carrying 5′-AN8-3′ barcodes with a 5′ phosphate and a 3′ blocking group. Barcoded RNAs were pooled and depleted of rRNA using the RiboZero rRNA depletion kit (Epicentre). These pools of barcoded RNAs were converted to Illumina cDNA libraries in 3 main steps: (i) reverse transcription of the RNA using a primer designed to the constant region of the barcoded adaptor; (ii) addition of a second adapter on the 3′ end of the cDNA during reverse transcription using SmartScribe RT (Clonetech) as described; (iii) PCR amplification using primers that target the constant regions of the 3′ and 5′ ligated adaptors and contain the full sequence of the Illumina sequencing adaptors. cDNA libraries were sequenced on Illumina HiSeq 2500. For the analysis of RNAtag-Seq data, reads from each sample in the pool were identified based on their associated barcode using custom scripts, and up to 1 mismatch in the barcode was allowed with the caveat that it did not enable assignment to more than one barcode. Barcode sequences were removed from reads, and the reads were mapped to the genome of the GenBank assembly of E. lenta A2 (Genome ID PPUL00000000) with Bowtie2 and feature counts derived using Rsubread (79). Differential expression analysis was carried out using DESeq2 with a significant threshold of FDR<0.1 and an absolute log2 fold change of 1. For preparation of the Manhattan plot (differential expression by gene location), scaffolds were concatenated and a pseudo base position used.

### Distribution of E. lenta A2 dadh Among Sequenced Genomes

The E. lenta A2 dopamine dehydroxylase protein sequence (accession # WP_086414988.1) was used as the query sequence for a pBLAST search of the NCBI non-redundant protein database (May 1, 2018). The 500 highest-scoring sequences were exported, but only the top sequences (down to ˜80% ID/E=0 score) were considered true dopamine dehydroxylase hits.

### Assessment of Conservation of dadh and Surrounding Genes Across Actinobacterial Library

Reads from genome sequencing were mapped to the reference A2000003 contig using Bowtie2 and filtered for a minimum mapping quality of 10. Variants were called when >80% of reads supported an alternate sequence. The phylogenetic tree of E. lenta strains was prepared previously.

### Conservation of Eggerthella lenta A2 dadh Among Actinobacterial Isolates and Alignment of Sequences

The Eggerthella lenta A2 dopamine dehydroxylase protein sequence was used as the query sequence for a pBLAST search of 26 previously sequenced Actinobacterial genomes (May 1, 2018). The genomes were loaded in Geneious (version 11) and BLASTP hits with an amino acid ID of >92% and e-value of 0 were considered dopamine dehydroxylase hits. These sequences were extracted and aligned using Jalview version 2.10.4, allowing for identification of the amino acid residue at position 506.

### Evaluation of the Effects of Tungstate and Molybdate on Dopamine Dehydroxylation in Cultures of E. lenta A2

Starter cultures of E. lenta A2 were grown over 48 hours in 10 mL BHI medium and then inoculated 1:100 into 200 μL BHI medium containing 500 μM dopamine and sodium tungstate (250 sodium molybdate (1000 or vehicle (water). Cultures were grown for 48 hours anaerobically at 37° C. and were harvested by centrifugation. Supernatants were dissolved 1:10 in LC-MS grade methanol and analyzed using LC-MS/MS Method A described above. Experiments were performed anaerobically, and cultures were grown in 96-well plates (VWR, catalog #29442-054).

### Anaerobic Activity-Based Purification of E. lenta A2 Dopamine Dehydroxylase

Protein purification: All experiments were performed under strictly anaerobic conditions at 4° C. Procedures outside the anaerobic chamber were performed in tightly sealed containers to prevent oxygen contamination. First, E. lenta A2 starter cultures were inoculated from single colonies into liquid BHI medium and were grown for 30 hours. Starter cultures were diluted 1:100 into 4 L of BHI medium containing 1% arginine and 10 mM formate and grown anaerobically at 37° C. for 17 hours. Cells were pelleted in 4 separate 1 liter bottles by centrifugation and each pellet was resuspended in 20 mL 20 mM

Tris pH 8 containing 4 mg/mL SIGMAFAST protease inhibitor cocktail. Resuspended cells were then lysed using sonication in an anaerobic chamber. The lysates were then clarified by centrifugation and the soluble fractions were subjected to two rounds of ammonium sulfate precipitation. During the precipitation, two of the four 20 mL clarified lysates were combined into a final volume of 40 mL, creating two 40 mL clarified lysates from the original 4 L culture. Solid ammonium sulfate was then dissolved in these lysates at a final concentration of 30% (w/v) and lysates were left for 1 hour and 20 minutes followed by centrifugation to pellet the precipitates. The supernatant was mixed with ammonium sulfate to achieve a final concentration of 40% (w/v) and left for 1 hour and 20 minutes. Following centrifugation, each pellet containing the precipitated proteins was re-dissolved in 20 mL 20 mM Tris pH 8 containing 0.5 M ammonium sulfate. The re-dissolved pellets were combined and the resulting 40 mL were injected onto an FPLC (Bio-Rad BioLogic DuoFlow System equipped with GE Life Sciences DynaLoop90) for hydrophobic interaction chromatography (HIC) using 5×1 mL HiTrap phenyl HP columns (GE Life Sciences, catalog #17135101). Fractions were eluted with a gradient of 0.5 M to 0 M ammonium sulfate (in 20 mM Tris pH 8) at a flow rate of 1 mL/min and were tested for activity using the assay described below. The majority of the dopamine dehydroxylase activity eluted around 0.05 M-0.1 M ammonium sulfate. Active fractions displaying >50% conversion of dopamine were combined and injected onto the FPLC system described above for anion exchange chromatography using a UNO Q1 column (Bio-Rad, catalog # 720-0001) at a flow-rate of 1 mL/min. Fractions were eluted using a gradient of 0 to 1 M NaCl in 20 mM Tris pH 8 and were tested for activity. The majority of the dopamine dehydroxylase activity eluted around 250 mM NaCl. Active fractions were combined and concentrated 20-fold using a spin concentrator with a 5 kDa cutoff. The concentrate was injected onto FPLC for size exclusion chromatography using an Enrich 24 mL column (Enrich SEC 650, 10*300 column, Bio-Rad, catalog #780-1650). Fractions were eluted over a 26 mL volume run isocratically in 20 mM Tris pH 8 containing 250 mM NaCl and were subjected to activity assays. Active fractions were run on SDS-PAGE to assess the presence of protein.

Activity assays: 50 μL fractions from FPLC were mixed, in the following order, with 1 electron donors (final concentration 1 mM each of methyl viologen, 1 mM diquat dibromide, 1 mM benzyl viologen, all dissolved in water), 2 μL sodium dithionite (2 mM final concentration, dissolved in water),), and 1μL dopamine (500 μM final concentration, dissolved in water),). The assay mixtures were left at room temperature in an anaerobic chamber for 12-14 hours to allow dopamine dehydroxylation to proceed, followed by dilution 1:20 into LC-MS grade methanol to stop the reaction. The diluted reactions were centrifuged to pellet any precipitates and the supernatant was analyzed by LC-MS using Method A described above.

Proteomics: Sample preparation, global proteomics—To 250 μL of fraction 5 shown in Figure S11 was added 3μL of 20 mM Tris(2-carboxyethyl)phopshine (TCEP, Sigma-Aldrich, catalog #75259) in 50 mM TEAB triethylammonium bicarbonate (TEAB, Sigma-Aldrich, catalog # T7408). The mixture was incubated at 37° C. for 1 hour in a sealed tube. The mixture was cooled to room temp for 10 minutes, followed by vortexing and centrifugation. To this mixture was added 3μL of freshly prepared 40 mM iodoacetamide Ultra (Sigma-Aldrich, catalog # I1149-5G) in 50 mM TEAB. The reaction mixture was incubated in a sealed tube for 1 hour at room temperature under tin foil to block light. 0.5 of trypsin (Promega, catalog # V5111) was added, and the mixture was incubated for 16 hours at 37° C. in a thermocycler. This sample was used for protein identification by LC-MS/MS, as described below. Sample preparation, gel band corresponding to dopamine dehydroxylase—The cut-out gel band was washed twice with 50% aqueous acetonitrile for 5 minuts followed by drying in a SpeedVac. The gel was then reduced with a volume sufficient to completely cover the gel pieces (100 μL) of 20 mM TCEP in 25 mM TEAB at 37° C. for 45 minutes. After cooling to room temp, the TCEP solution was removed and replaced with the same volume of 10 mM iodoacetamide Ultra (Sigma) in 25 mM TEAB and kept in the dark at room temperature for 45 minutes. Gel pieces were washed with 200 μL of 100 mM TEAB (10 minutes). The gel pieces were then shrunk with acetonitrile. The liquid was then removed followed by swelling with the 100 mM TEAB again and dehydration/shrinking with the same volume of acetonitrile. All of thee liquid was removed, and the gel was completely dried in a SpeedVac for ˜20 minutes. 0.06 μg/5 μL of trypsinin 50 mM TEAB was added to the gel pieces and the mixture was placed in a thermomixer at 37° C. for about 15 min. 50 μL of 50 mM TEAB was added to the gel slices. The samples were vortexed, centrifuged, and placed back in the thermomixer overnight. Samples were digested overnight at 37° C. Peptides were extracted with 50 μL 20 mM TEAB for 20 min and 1 change of 50 μL 5% formic acid in 50% acetonitrile at room temp for 20 minutes while in a sonicator. All extracts obtained were pooled into an HPLC vial and were dried using a SpeedVac to the desired volume (−50 μL). This sample was used for protein identification by LC-MS/MS, as described below.

Mass spectrometry: Each sample was submitted for a single LC-MS/MS experiment that was performed on an LTQ Orbitrap Elite (Thermo Fischer) equipped with a Waters (Milford, Mass.) NanoAcquity HPLC pump. Peptides were separated using a 100 μm inner diameter microcapillary trapping column packed first with approximately 5 cm of C18 Reprosil resin (5 μm , 100 Å, Dr. Maisch GmbH, Germany) followed by ˜20 cm of Reprosil resin (1.8 μm, 200 Å, Dr. Maisch GmbH, Germany). Separation was achieved through applying a gradient of 5-27% ACN in 0.1% formic acid over 90 min at 200 nL min ⁱ. Electrospray ionization was enabled through applying a voltage of 1.8 kV using a home-made electrode junction at the end of the microcapillary column and sprayed from fused silica pico tips (New Objective, Mass.). The LTQ Orbitrap Elite was operated in data-dependent mode for the mass spectrometry methods. The mass spectrometry survey scan was performed in the Orbitrap in the range of 395-1,800 m/z at a resolution of 6×10⁴, followed by the selection of the twenty most intense ions (TOP20) for CID-MS2 fragmentation in the Ion trap using a precursor isolation width window of 2 m/z, AGC setting of 10,000, and a maximum ion accumulation of 200 ms. Singly charged ion species were not subjected to CID fragmentation. Normalized collision energy was set to 35 V and an activation time of 10 ms. Ions in a 10 ppm m/z window around ions selected for MS2 were excluded from further selection for fragmentation for 60 seconds. The same TOP20 ions were subjected to HCD MS2 event in Orbitrap part of the instrument. The fragment ion isolation width was set to 0.7 m/z, AGC was set to 50,000, the maximum ion time was 200 ms, normalized collision energy was set to 27 V and an activation time of 1 ms for each HCD MS2 scan.

Mass spectrometry data analysis: Raw data were submitted for analysis in Proteome Discoverer 2.1.0.81 (Thermo Scientific) software. Assignment of MS/MS spectra were performed using the Sequest HT algorithm by searching the data against a protein sequence database including a custom database from Eggerthella lenta A2 and entries from the Human Uniprot database (16,768 proteins from SwissProt and 62,460 proteins from TrEMBL for a total of 79,228 protein forms, 2015) and other sequences such as human keratins and common lab contaminants. Sequest HT searches were performed using a 20 ppm precursor ion tolerance and requiring each peptide's N-/C-termini to adhere with trypsin protease specificity, while allowing up to two missed cleavages. Cysteine carbamidomethyl (+57.021) was set as a static modification while methionine oxidation (+15.99492 Da) was set as a variable modification. A MS2 spectra assignment false discovery rate (FDR) of 1% on protein level was achieved by applying the target-decoy database search. Filtering was performed using a Percolator (64 bit version). For quantification, a 0.02 m/z window centered on the theoretical m/z value of each the six reporter ions and the intensity of the signal closest to the theoretical m/z value was recorded. Reporter ion intensities were exported in result file of Proteome Discoverer 2.1 search engine as an excel tables.

### Co-Culturing of E. faecalis and E. lenta A2

E. faecalis MMH594 WT or tyrDC mutant or E. lenta A2 or E. lenta DSM2243 were grown anaerobically for 48 hours from single colonies in BHI medium at 37° C. These starter cultures were normalized to an OD₆₀₀=0.1 by dilution into fresh BHI medium. 10 μL of each normalized starter culture was diluted in 5 mL of BHI medium containing 0.75% (w/v) arginine and either 1 mM d₃-phenyl-L-dopa or dopamine. Cultures were grown for 48 hours at 37° C. and harvested by centrifugation. Culture supernatants were diluted 1:10 in LC-MS grade methanol and were centrifuged to pellet precipitates. Supernatants were analyzed by LC-MS using Method C described above.

### Ex Vivo Assays With d₃-Phenyl-L-Dopa

Fecal slurries from neurologically healthy human donors or Parkinson's patients were prepared as described in the ‘enrichment culturing’ section above. These slurries were diluted to a final volume of 300 μL in 20% glycerol and then aliquoted into individual tubes, flash-frozen in liquid nitrogen and stored at −80° C., allowing for experiments to be repeated with the same fecal sample multiple times without freeze-thawing. These slurries were diluted 1:100 into 5 mL MEGA or BHI media containing 1 mM d₃-phenyl-L-dopa with or without carbidopa (2 mM) or AFMT (200-250 μM) in triplicate and were grown anaerobically at 37° C. for 72 hours. Cultures were harvested by centrifugation and culture supernatants were diluted 1:10 in LC-MS grade methanol, followed by another round of centrifugation to pellet precipitates. Supernatants were analyzed by LC-MS using Method C described above.

### Gain of Function Assays in Complex Human Gut Microbiota Samples

E. faecalis MMH594 WT or tyrDC mutant or E. lenta A2 or E. lenta DSM2243 were grown anaerobically for 48 hours from single colonies in BHI medium at 37° C. These starter cultures were normalized to an OD₆₀₀=0.1 by dilution into fresh BHI medium. 10 μL of each normalized starter culture was diluted in MEGA medium containing 1 mM d₃-phenyl-L-dopa. At the time of addition of E. faecalis and/or E. lenta, fecal samples determined to be non-metabolizers with regard to L-dopa were inoculated into the medium as described above and were grown anaerobically for 72 hors at 37° C. Cultures were harvested by centrifugation and culture supernatants were diluted 1:10 in LC-MS grade methanol, followed by another round of centrifugation to pellet precipitates. Supernatants were analyzed by LC-MS using Method C described above.

### qPCR Assays

gDNA was extracted from the culture pellets generated in the experiments described above (‘Ex vivo assays with d₃-phenyl-L-dopa’) using the DNeasy UltraClean Microbial Kit. 2 ng of the extracted DNA from each culture was used for qPCR assays containing 10 μL of iTaq Universal SYBRgreen Supermix (Bio-rad, catalog 3: 1725121), 7 μL of water, and 10 μM each of forward and reverse primers. PCR was performed on a CFX96 Thermocycler (Bio-Rad), using the following program: initial denaturation at 95° C. for 5 minutes 34 cycles of 95° C. for 1 min, 60° C. for 1 min, 72° C. for 1 min. The program ended with a final extension at 34° C. for 5 mins. The primers used were: 16S primers for E. faecalis (81): CGCTTCTTTCCTCCCGAGT and GCCATGCGGCATAAACTG; 16S primers for E. lenta (45): CAGCAGGGAAGAAATTCGAC and TTGAGCCCTCGGATTAGAGA; primers for dopamine dehydroxylase: GAGATCTGGTCCACCGTCAT and AGTGGAAGTACACCGGGATG; primers for tyrDC (47): CGTACACATTCAGTTGCATGGCAT and ATGTCCTACTCCTCCTCCCATTTG.

### Ex Vivo Assays for Dopamine Dehydroxylation and Sanger Sequencing

Fecal slurries from human donors were prepared as described in the ‘enrichment culturing’ section above. These slurries were diluted 1:100 into BHI medium containing 1% arginine (w/v) and 10 mM formate as well as 500 μM dopamine. Cultures were grown anaerobically at 37° C. for 72 hours. Cultures were harvested by centrifugation, and culture supernatants were diluted 1:10 in LC-MS grade methanol, followed by another round of centrifugation to pellet precipitates. Supernatants were analyzed by LC-MS using Method A described above. gDNA was extracted from the culture pellet using the DNeasy UltraClean Microbial Kit. 1 ng of the extracted DNA from each culture was used for PCR assays containing 10 μL of Phusion High-Fidelity PCR Master mix with HF buffer (NEB, catalog # M0531L), 7μL of water, and 10 μM each of forward and reverse primers. The primers used to amplify the full-length dopamine dehydroxylase from these samples were ATGGGTAACCTGACCATG and TTACTCCCTCCCTTCGTA. PCR was performed on a C1000 Touch Thermocycler (Bio-Rad), using the following program: initial denaturation at 98° C. for 30 s, 34 cycles of 98° C. for 10 s, 61° C. for 15 s, 72° C. for 2.5 mins. The program ended with a final extension at 72° C. for 5 mins. Amplicons were purified using the Illustra GFX PCR DNA and Gel Band Purification Kit (GE healthcare, catalog #28-9034-70) and were sequenced using Sanger sequencing (Eton Biosciences) for the region containing the SNP at position 506 using primers GGGGTGTCCATGTTGCCGGT and ACCGGCTACGGCAACGGC. Sequence chromatograms were analyzed in Ape Plasmid Editor (version 2.0.47), and the single nucleotide polymorphism (SNP) at position 506 was called by visual inspection compared to results obtained from control cultures of E. lenta strains. Samples where two peaks existed at position 506 were determined to have a mix of SNPs present in the sample and were removed from analysis.

### Metagenomic Analysis of E. lenta/dadh and E. faecalis/tyrDC Abundance and Prevalence Across Human Patients

A curated collection of human gut microbiomes representing 1870 individuals (82), was used to correlate the abundances of E. lenta/dadh and Enterococcus/tyrDC (Pearson correlation, R). Prevalence was estimated as a function of rolling minimum abundance cut off. SNP analysis was carried out as before (41), by mapping reads from a set of 96 samples with high E. lenta genome coverage to the reference genome of A2 (Assembly accession GCA_ 003340125.1) after quality filtering with FastP and using Bowtie 2.3.4.1 and SAMtools 1.9.

### MTT Assay for HeLa Cell Viability in the Presence of AFMT

HeLa cells were seeded into 96-well plates at a density of 1×10⁵ cells per well in 100 μL of growth medium [(DMEM medium supplemented with 10% FBS (2 mL) and 1× Antibiotic-Antimycotic (100× stock, Invitrogen))] and incubated at 37° C. in a 5% CO₂ incubator for 1 day. Wells containing growth medium only were used as background controls. Cells were treated with various concentrations of AFMT in quadruplicate. Two days post treatment, 20 μL of CellTiter 96® AQueous One Solution Reagent (Promega) was added to each well. The plates were incubated at 37° C. in a 5% CO₂ incubator for 2 hours followed by measurement of absorbance at 490 nm using a Synergy HTX Multi-Mode Microplate Reader (BioTek). To calculate relative cell viability, the readings for each compound concentration were subtracted from the background controls and normalized to vehicle controls

### Pharmacokinetic Experiment in Gnotobiotic Mice

Germ-free male BALB/c mice aged 7-12 weeks (n=11) were colonized with 1×10⁸ CFU E. faecalis MMH594 (in 100 μL Brain Heart Infusion media) and randomly selected to receive 10 mg/kg AFMT or vehicle control (0.25% carboxymethylcellulose) balancing age across groups. Mice were administered AFMT or vehicle 1 hour before a second dose co-administered with 10 mg/kg d₃-phenyl-L-dopa and 30 mg/kg Carbidopa. Blood was collected from the tail vein at Time=0 min, 15 min, 30 min, 60 min, 90 min, and 120 min for determination of serum L-dopa.

### Extraction and Analysis of L-Dopa From Tail Vein Blood

All manipulations were performed in 1.5 mL Eppendorf tubes. 5 μL tail-vein blood was added to a mixture of 25 μL 0.2 M sodium acetate buffer (pH 5.5, containing 12.5 μM unlabeled L-dopa as the internal standard) and 50 μL ice-cold methanol (containing 35 μM EDTA and 0.1% (w/v) ascorbic acid as anti-oxidants). The mixture was vortexed and left on ice for 5 minutes to precipitate proteins, whereby 100 μL chloroform was added to remove serum lipids. The mixture was vortexed until the solution appeared homogenous and samples were then left at −80° C. for 10 minutes. Organic and aqueous layers were then separated using centrifugation for 10 minutes, and the top aqueous layer was then carefully removed (50 μL) and transferred to a new Eppendorf tube. This aqueous layer was evaporated using a Genevac (EZ-2 Elite personal evaporator, HPLC setting with 30 mins each for the first and second phases, no heating). The dried residue was then resuspended in 15 μL MilliQ water (containing 0.1% formic acid) by vortexing. Samples were spun down to pellet potential particulates and 10 μL of the resulting supernatant was injected for LC-MS analysis using Method H described above. Quantification was performed by normalization of the d₃-phenyl-L-dopa analyte peak area to the peak area of the unlabeled L-dopa internal standard.

TABLE 1

| | Tyrosine | L-dopa |
|---|---|---|
| Vₘₐₓ (μM/s) | 6.363 ± 0.102 | 5.553 ± 0.165 |
| K_{M} (μM) | 314.9 ± 15.5 | 1475 ± 79.28 |
| k_{cat} (1/s) | 63.63 ± 1.02 | 55.53 ± 2.98 |
| k_{cat}/K_{M} (1/μM*s) | 2.02 ± 0.14 *10⁵ | 3.77 ± 0.23 *10⁴ |

Michaelis-Menten parameters determined for TyrDC. Data represent the best-fit values and their associated standard error (n=3 replicates).

TABLE 2

| | | | p-value |
|---|---|---|---|
| Locus tag | Annotation | log2FoldChange | (FDR < 0.1) |
| 237-Elenta- | 4Fe—4S dicluster | 11.43265558 | 1.11E−26 |
| A2_00534 | domain-containing | | |
| | protein | | |
| 237-Elenta- | Molybdopterin | 11.32638074 | 7.46E−79 |
| A2_00535 | dinucleotide-binding | | |
| | protein | | |
| 237-Elenta- | Hypothetical protein | 10.7757051 | 1.60E−47 |
| A2_00533 | | | |
| 237-Elenta- | Uncharacterized | 9.648691059 | 7.67E−19 |
| A2_00540 | component of | | |
| | anaerobic dehydrogenase | | |
| 237-Elenta- | Hypothetical protein | 9.064741704 | 1.09E−16 |
| A2_00539 | | | |
| 237-Elenta- | 4Fe—4S binding | 7.74321557 | 4.18E−18 |
| A2_00542 | protein | | |
| 237-Elenta- | 4Fe—4S ferredoxin | 7.452293617 | 2.49E−44 |
| A2_00543 | | | |
| 237-Elenta- | 4Fe—4S ferredoxin | 7.108180453 | 6.16E−20 |
| A2_00541 | | | |
| 237-Elenta- | Hypothetical protein | 5.687034137 | 4.53E−109 |
| A2_00538 | | | |
| 237-Elenta- | Hypothetical protein | 4.974609256 | 5.23E−48 |
| A2_00544 | | | |
| 237-Elenta- | Energy-coupling factor | 2.430668122 | 5.71E−28 |
| A2_02490 | ABC transporter | | |
| | ATP-binding protein | | |
| 237-Elenta- | ABC transporter | 2.079536895 | 2.58E−23 |
| A2_02489 | ATP-binding protein | | |
| 237-Elenta- | EEnergy-coupling | 1.749835331 | 1.58E−18 |
| A2_02488 | factor transporter | | |
| | transmembrane | | |
| | protein EcfT | | |
| 237-Elenta- | Hypothetical protein | 1.601691749 | 2.21E−20 |
| A2_02491 | | | |
| 237-Elenta- | TrpB-like pyridoxal | 1.228094756 | 7.69E−52 |
| A2_01052 | phosphate-dependent | | |
| | enzyme | | |

Genes upregulated in E. lenta A2 when grown with 500 μM dopamine relative to a vehicle control in BHI medium containing 1% (w/v) arginine and 10 mM formate. The catalytic subunit of the dopamine dehydroxylase is highlighted in red.

TABLE 3

| | | | # | MW |
|---|---|---|---|---|
| Locus tag | Annotation | Coverage | AAs | [kDa] |
| 237-Elenta- | Molybdopterin dinucleotide | 58.41 | 1017 | 115 |
| A2_00535 | binding region | | | |
| 237-Elenta- | Heat shock protein Hsp90 | 31.42 | 646 | 72.6 |
| A2_00242 | | | | |
| 237-Elenta- | Pyruvate ferredoxin/flavodoxin | 33.47 | 1180 | 127.6 |
| A2_02071 | oxidoreductase | | | |
| 237-Elenta- | Nickel-dependent hydrogenase | 5.96 | 554 | 60.7 |
| A2_01108 | large subunit | | | |

Proteomics identification of bands in active size exclusion fraction (lane 5 in FIG. 21) during purification of the dopamine dehydroxylase from E. lenta A2. The catalytic subunit of the dopamine dehydroxylase is highlighted in red.

TABLE 4

| | | | Annotation | Query | | | Accession |
|---|---|---|---|---|---|---|---|
| Class | Order | Family | [species] | cover | E-value | % ID | number |
| Coriobacteriia | Eggerthellales | Eggerthellaceae | molybdopterin | 100% | 0 | 100% | WP_086414988.1 |
| | | | dinucleotide- | | | | |
| | | | binding protein | | | | |
| | | | [Eggerthella lenta] | | | | |
| Coriobacteriia | Eggerthellales | Eggerthellaceae | molybdopterin | 100% | 0 | 99% | WP_015759999.1 |
| | | | dinucleotide- | | | | |
| | | | binding protein | | | | |
| | | | [Eggerthella lenta | | | | |
| | | | DSM2243] | | | | |
| Coriobacteriia | Eggerthellales | Eggerthellaceae | molybdopterin | 100% | 0 | 99% | WP_009608130.1 |
| | | | dinucleotide- | | | | |
| | | | binding protein | | | | |
| | | | [Eggerthella sp. | | | | |
| | | | HGA1] | | | | |
| Coriobacteriia | Eggerthellales | Eggerthellaceae | molybdopterin | 100% | 0 | 99% | WP_035584143.1 |
| | | | dinucleotide- | | | | |
| | | | binding protein | | | | |
| | | | [Eggerthella lenta | | | | |
| | | | 1_1_60AFAA] | | | | |
| Coriobacteriia | Eggerthellales | Eggerthellaceae | molybdopterin | 100% | 0 | 99% | WP_009306100.1 |
| | | | dinucleotide- | | | | |
| | | | binding protein | | | | |
| | | | [Eggerthella sp. | | | | |
| | | | 1_3_56FAA] | | | | |
| Coriobacteriia | Eggerthellales | Eggerthellaceae | molybdopterin | 100% | 0 | 98% | WP_101721531.1 |
| | | | dinucleotide- | | | | |
| | | | binding protein | | | | |
| | | | [Eggerthella | | | | |
| | | | timonensis] | | | | |
| Coriobacteriia | Eggerthellales | Eggerthellaceae | molybdopterin | 100% | 0 | 94% | WP_087197552.1 |
| | | | dinucleotide- | | | | |
| | | | binding protein | | | | |
| | | | [Gordonibacter | | | | |
| | | | sp. An232A] | | | | |
| Coriobacteriia | Eggerthellales | Eggerthellaceae | molybdopterin | 100% | 0 | 93% | WP_103263115.1 |
| | | | dinucleotide- | | | | |
| | | | binding protein | | | | |
| | | | [Rubneribacter | | | | |
| | | | badeniensis] | | | | |
| Coriobacteriia | Eggerthellales | Eggerthellaceae | molybdopterin | 100% | 0 | 93% | WP_087194795.1 |
| | | | dinucleotide- | | | | |
| | | | binding protein | | | | |
| | | | [Gordonibacter | | | | |
| | | | sp. An230] | | | | |
| Coriobacteriia | Eggerthellales | Eggerthellaceae | molybdopterin | 100% | 0 | 82% | WP_012799829.1 |
| | | | dinucleotide- | | | | |
| | | | binding protein | | | | |
| | | | [Slackia | | | | |
| | | | heliotrinireducens] | | | | |

List of predicted dopamine dehydroxylases as retrieved from the non-redundant NCBI database at a threshold of 82% amino acid identity.

TABLE 5

| | Query | | % Amino |
|---|---|---|---|
| Strain | cover | E-value | Acid ID |
| DSM11767 | 100.00% | 0 | 100.0% |
| AB12n2 | 100.00% | 0 | 100.0% |
| A2 | 100.00% | 0 | 100.0% |
| RC46F | 100.00% | 0 | 99.9% |
| MRn12 | 100.00% | 0 | 99.9% |
| DSM18163 | 100.00% | 0 | 99.9% |
| DSM15644 | 100.00% | 0 | 99.9% |
| CC86D54 | 100.00% | 0 | 99.9% |
| AN51LG | 100.00% | 0 | 99.9% |
| AB8n2 | 100.00% | 0 | 99.9% |
| 28b | 100.00% | 0 | 99.9% |
| 22C | 100.00% | 0 | 99.9% |
| 16A | 100.00% | 0 | 99.9% |
| 14A | 100.00% | 0 | 99.9% |
| 11c | 100.00% | 0 | 99.9% |
| HGA1 | 100.00% | 0 | 99.7% |
| 1160FAA | 100.00% | 0 | 99.6% |
| 1160AFAA | 100.00% | 0 | 99.6% |
| 326IFAA | 100.00% | 0 | 99.6% |
| 1356FAA | 100.00% | 0 | 98.9% |
| Valencia | 100.00% | 0 | 98.3% |
| CC82BHI2 | 100.00% | 0 | 98.3% |
| CC75D52 | 100.00% | 0 | 98.3% |
| W1BHI6 | 100.00% | 0 | 98.1% |
| RC22A | 100.00% | 0 | 93.2% |
| DSM16107 | 100.00% | 0 | 92.1% |

List of predicted dopamine dehydroxylases as retrieved from a custom database of 28 Actinobacterial isolates (34) at a threshold of 92% amino acid identity.

TABLE 6

| | | log2 Fold | p-value |
|---|---|---|---|
| Locus tag | Annotation | Change | (FDR < 0.1) |
| Eggerthella_{—}lenta_DSM2243_00482 | Molydopterin dinucleotide binding region | 3.641149985 | 1.44E−163 |
| Eggerthella_{—}lenta_DSM2243_00480 | Hypothetical_protein | 3.140977549 | 3.76E−119 |
| Eggerthella_{—}lenta_DSM2243_00481 | 4Fe—4S ferredoxin | 2.73148694 | 2.63E−89 |
| Eggerthella_{—}lenta_DSM2243_00485 | Hypothetical protein | 1.668195003 | 7.10E−34 |
| Eggerthella_{—}lenta_DSM2243_00488 | 4Fe—4S ferredoxin | 1.478077832 | 2.12E−27 |
| Eggerthella_{—}lenta_DSM2243_00489 | Hypothetical protein | 1.395767311 | 6.78E−25 |
| Eggerthella_{—}lenta_DSM2243_00582 | Ferrous iron transport protein | 1.097665786 | 1.74E−14 |

Genes upregulated in E. lenta DSM2243 when grown with 500 μM dopamine relative to a vehicle control in BHI medium containing 1% (w/v) arginine and 10 mM formate. The catalytic subunit of the dopamine dehydroxylase is highlighted in red.

TABLE 7

| | | log2 Fold | p-value |
|---|---|---|---|
| Locus tag | Annotation | Change | (FDR < 0.1) |
| Eggerthella_{—}lenta_28b_02709 | Molydopterin dinucleotide binding region | 5.339268092 | 6.01E−86 |
| Eggerthella_{—}lenta_28b_02711 | Hypothetical_protein | 4.930759063 | 7.41E−65 |
| Eggerthella_{—}lenta_28b_02710 | 4Fe—4S ferredoxin | 3.020280491 | 2.27E−19 |
| Eggerthella_{—}lenta_28b_02706 | Hypothetical protein | 2.878320413 | 3.28E−18 |
| Eggerthella_{—}lenta_28b_02701 | 4Fe—4S ferredoxin | 2.611526487 | 2.50E−14 |
| Eggerthella_{—}lenta_28b_02708 | Transcriptional regulator, LuxR family | 2.06813492 | 9.27E−09 |

Genes upregulated in E. lenta 28b when grown with 500 μM dopamine relative to a vehicle control in BHI medium containing 1% (w/v) arginine and 10 mM formate (log2foldchange>2). The catalytic subunit of the dopamine dehydroxylase is highlighted in red.

TABLE 8

| | Carbidopa | AFMT |
|---|---|---|
| TyrDC | 52 ± 0.06 | 4.71 ± 0.01 |
| AADC | 0.21 ± 0.01 | 20% inhibition at solubility |
| | | limit of 700 μM |
| E. faecalis | 50% inhibition at | 1.41 ± 0.01 |
| MMH594 cells | solubility limit of 2 mM | |

Inhibition parameters determined for carbidopa and AFMT. IC₅₀ and EC₅₀ values (in μM) represent the best-fit values and their associated standard error (n=3 replicates).

### Data S1

Results from BLASTP of L. brevis TyrDC (UniProt accession, B8V35) against Human Microbiome Project (HMP) Reference genomes.

### Incorporation by Reference

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### Equivalents

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a condition in a subject, comprising administering an agent that inhibits the activity of or decreases the levels of an L-dopa decarboxylase conjointly with levodopa (L-dopa).

2. The method of claim 1, wherein the L-dopa decarboxylase is tyrosine decarboxylase (TyrDC).

3. The method of claim 2, wherein the agent preferentially inhibits the activity of or decreases the level of a TyrDC over amino acid decarboxylase (AADC).

4. The method of any one of claims 1 to 3, wherein the condition is Parkinsonism.

5. The method of any one of claims 1 to 4, wherein the condition is Parkinson's disease.

6. The method of any one of claims 1 to 4, wherein the condition is corticobasal degeneration (CBD)

7. The method of any one of claims 1 to 4, wherein the condition is dementia with Lewy bodies (DLB).

8. The method of any one of claims 1 to 4, wherein the condition is essential tremor.

9. The method of any one of claims 1 to 4, wherein the disease or disorder is multiple system atrophy (MSA)

10. The method of any one of claims 1 to 4, wherein the condition is progressive supranuclear palsy (PSP).

11. The method of any one of claims 1 to 4, wherein the condition is vascular (arteriosclerotic) parkinsonism.

12. The method of any one of claims 1 to 4, wherein the condition is Parkinson's-like symptoms that develop after encephalitis.

13. The method of any one of claims 1 to 3, wherein the condition is injury to the nervous system caused by carbon monoxide poisoning.

14. The method of any one of claims 1 to 3, wherein the condition is injury to the nervous system caused by manganese poisoning.

15. The method of any one of claims 1 to 14, wherein the agent is a small molecule.

16. The method of claim 15, wherein the agent is (S)-α-Fluoromethyltyrosine (AFMT).

17. The method of any one of claims 1 to 14, wherein the agent is an interfering nucleic acid specific for a RNA product of a gene encoding a TyrDC or fragment thereof.

18. The method of claim 17, wherein the interfering nucleic acid is a siRNA.

19. The method of claim 17, wherein the interfering nucleic acid is a shRNA.

20. The method of claim 17, wherein the interfering nucleic acid is a miRNA.

21. The method of any one of claims 1 to 14, wherein the agent is a CRISPR, a TALEN, or a Zinc-finger nuclease.

22. The method of claim 21, wherein the agent is a CRISPR single guide RNA (sgRNA).

23. The method of claim 17, wherein the interfering nucleic acid is a peptide nucleic acid.

24. The method of any one of claims 1 to 14, wherein the agent is an antibody specific for a TyrDC protein.

25. The method of claim 24, wherein the antibody is a polyclonal antibody.

26. The method of claim 24, wherein the antibody is a monoclonal antibody.

27. The method of claim 24, wherein the antibody is a chimeric antibody.

28. The method of claim 24, wherein the antibody is a humanized antibody.

29. The method of claim 24, wherein the antibody is an antibody fragment.

30. The method of any one of claims 1 to 14, wherein the agent is a peptide that specifically binds to a TyrDC protein or fragment thereof.

31. The method of any one of the preceding claims, wherein the agent and levodopa are administered in one composition.

32. The method of any one of the preceding claims, wherein the agent and levodopa are administered simultaneously or sequentially.

33. The method of any one of claims 1 to 30, wherein the agent and levodopa are administered in different compositions.

34. The method of any one of the preceding claims, further comprising administering carbidopa or benserazide to the subject.

35. The method of any one of the preceding claims, further comprising administering an agent that to the subject that inhibits the activity of or decreases the levels of an enzyme that dehydroxylates dopamine.

36. The method claim 35, wherein the enzyme that dehydroxylates dopamine is bis-molybdopterin guanine dinucleotide cofactor (moco)-containing enzyme.

37. A method of treating Parkinson's Disease in a subject, comprising administering an agent to the subject that inhibits the activity of or decreases the levels of TyrDC conjointly with levodopa.

38. A method of treating or preventing symptoms resulting from dopaminergic neuron loss in a subject in need thereof, comprising administering an agent to the subject that inhibits the activity of or decreases the levels of TyrDC conjointly with levodopa.

39. The method of claim 37 or 38, wherein the agent preferentially inhibits the activity of or decreases the level of TyrDC over AADC.

40. The method of any one of claims 37 to 39, wherein the agent is a small molecule.

41. The method of claim 40, wherein the agent is AFMT.

42. The method of any one of claims 37 to 39, wherein the agent is an interfering nucleic acid specific for a RNA product of a gene encoding for TyrDC or fragment thereof.

43. The method of claim 42, wherein the interfering nucleic acid is a siRNA.

44. The method of claim 42, wherein the interfering nucleic acid is a shRNA.

45. The method of claim 42, wherein the interfering nucleic acid is a miRNA.

46. The method of any one of claims 37 to 39, wherein the agent is a CRISPR, a TALEN, or a Zinc-finger nuclease.

47. The method of claim 46, wherein the agent is a CRISPR single guide RNA (sgRNA).

48. The method of any one of claims 37 to 39, wherein the interfering nucleic acid is a peptide nucleic acid.

49. The method of any one of claims 37 to 39, wherein the agent is an antibody specific for a TyrDC protein.

50. The method of claim 49, wherein the antibody is a polyclonal antibody.

51. The method of claim 49, wherein the antibody is a monoclonal antibody.

52. The method of claim 49, wherein the antibody is a chimeric antibody.

53. The method of claim 49, wherein the antibody is a humanized antibody.

54. The method of claim 49, wherein the antibody is an antibody fragment.

55. The method of any one of claims 37 to 39, wherein the agent is a peptide that specifically binds to a TyrDC protein or fragment thereof.

56. The method of any one of claims 37 to 55, wherein the agent and levodopa are administered in one composition.

57. The method of any one of claims 37 to 55, wherein the agent and levodopa are administered simultaneously.

58. The method of any one of claims 37 to 55, wherein the agent and levodopa are administered in different compositions.

59. The method of any one of claims 37 to 55, wherein the agent and the levodopa are administered sequentially.

60. The method of any one of claims 35 to 59, further comprising administering an agent that to the subject that inhibits the activity of or decreases the levels of an enzyme that dehydroxylates dopamine.

61. The method claim 60, wherein the enzyme that dehydroxylates dopamine is bis-molybdopterin guanine dinucleotide cofactor (moco)-containing enzyme.

62. The method of any one of claims 35 to 61, further comprising administering carbidopa or benserazide to the subject.

63. The method of any one of claims 1 to 62, wherein the agent decreases the level of or inhibits the activity of a TyrDC protein by at least 10%.

64. The method of any one of claims 1 to 63, wherein the agent decreases the level of or inhibits the activity of a TyrDC protein by at least 20%.

65. The method of any one of claims 1 to 64, wherein the agent decreases the level of or inhibits the activity of a TyrDC protein by at least 30%.

66. The method of any one of claims 1 to 65, wherein the agent decreases the level of or inhibits the activity of a TyrDC protein by at least 40%.

67. The method of any one of claims 1 to 66, wherein the agent decreases the level of or inhibits the activity of a TyrDC protein by at least 50%.

68. The method of any one of claims 1 to 67, wherein the agent decreases the level of or inhibits the activity of a TyrDC protein by at least 75%.

69. The method of any one of claims 1 to 68, wherein the agent decreases the level of or inhibits the activity of a TyrDC protein by at least 90%.

70. The method of any one of claims 1 to 69, wherein the agent decreases the level of or inhibits the activity of a TyrDC protein by at least 99%.

71. The method of any one of the preceding claims, wherein the agent is administered to the subject systemically.

72. The method any one of claims 1 to 71, wherein the agent is administered intravenously.

73. The method any one of claims 1 to 71, wherein the agent is administered subcutaneously.

74. The method any one of claims 1 to 71, wherein the agent is administered intramuscularly.

75. The method any one of claims 1 to 71, wherein the agent is administered orally.

76. The method any one of claims 1 to 71, wherein the agent is administered locally.

77. The method of any one of the preceding claims, wherein the agent is administered to the subject in a pharmaceutically acceptable formulation.

78. An in-vitro method of determining whether an agent is a therapeutic agent for Parkinson's Disease comprising determining whether the test agent decreases the levels of or inhibits the activity of a TyrDC enzyme, wherein the test agent is determined to be a therapeutic agent for the treatment of Parkinson's Disease if the test agent decreases the levels of or inhibits the activity of a TyrDC enzyme.

79. The method of claim 78, wherein the test agent is a member of a library of test agents.

80. The method of claim 78 or 79, wherein the test agent is an interfering nucleic acid.

81. The method of claim 78 or 79, wherein the test agent is a peptide.

82. The method of claim 78 or 79, wherein the test agent is a small molecule.

83. The method of claim 78 or 79, wherein the test agent is an antibody.

84. The method of any one of claims 78 to 83, wherein the agent decreases the level of or inhibits the activity of the TyrDC enzyme by at least 10%.

85. The method of any one of claims 78 to 83, wherein the agent decreases the level of or inhibits the activity of the TyrDC enzyme by at least 20%.

86. The method of any one of claims 78 to 83, wherein the agent decreases the level of or inhibits the activity of the TyrDC enzyme by at least 30%.

87. The method of any one of claims 78 to 83, wherein the agent decreases the level of or inhibits the activity of the TyrDC enzyme by at least 40%.

88. The method of any one of claims 78 to 83, wherein the agent decreases the level of or inhibits the activity of the TyrDC enzyme by at least 50%.

89. The method of any one of claims 78 to 83, wherein the agent decreases the level of or inhibits the activity of the TyrDC enzyme by at least 75%.

90. The method of any one of claims 78 to 83, wherein the agent decreases the level of or inhibits the activity of the TyrDC enzyme by at least 95%.

91. A method of treating a condition in a subject, comprising administering a composition that inhibits the activity of or decreases the levels of a bacteria that expresses a PLP-dependent tyrosine decarboxylase (TyrDC) or a TyrDC homolog conjointly with levodopa.

92. The method of claim 91, wherein the bacteria that expresses a PLP-dependent tyrosine decarboxylase is Enterococcus faecalis.

93. The method of claim 91, wherein the bacteria that expresses a PLP-dependent tyrosine decarboxylase is Enterococcus faecium.

94. The method of any one of claims 91 to 93, wherein the condition is Parkinsonism.

95. The method of any one of claims 91 to 94, wherein the condition is Parkinson's disease.

96. The method of any one of claims 91 to 94, wherein the condition is corticobasal degeneration (CBD)

97. The method of any one of claims 91 to 94, wherein the condition is dementia with Lewy bodies (DLB).

98. The method of any one of claims 91 to 94, wherein the condition is essential tremor.

99. The method of any one of claims 91 to 94, wherein the disease or disorder is multiple system atrophy (MSA)

100. The method of any one of claims 91 to 94, wherein the condition is progressive supranuclear palsy (PSP).

101. The method of any one of claims 91 to 94, wherein the condition is vascular (arteriosclerotic) parkinsonism.

102. The method of any one of claims 91 to 94, wherein the condition is Parkinson's-like symptoms that develop after encephalitis.

103. The method of any one of claims 91 to 93, wherein the condition is injury to the nervous system caused by carbon monoxide poisoning.

104. The method of any one of claims 91 to 93, wherein the condition is injury to the nervous system caused by manganese poisoning.

105. The method of any one of claims 91 to 104, wherein the method further comprises administering a composition that inhibits the activity of or decreases the levels of a bacteria that expresses a molybdenum-dependent enzyme.

106. The method of claim 105, wherein the bacteria that expresses a molybdenum-dependent enzyme is Eggerthella lenta.
